(19) United States
(12) Patent Application Publication
(10) Pub. No.: US 2025/0352505 A1
(43) Pub. Date: Nov. 20, 2025

(54) COMPOSITIONS AND METHODS OF TREATING RESPIRATORY SYNCYTIAL VIRUS

(71) Applicants: University of South Florida, Tampa, FL (US); The UAB Research Foundation, Birmingham, AL (US)

(72) Inventors: Bill J. Baker, Temple Terrace, FL (US); Joe Bracegirdle, New South Wales (AU); Michael N. Teng, Tampa, FL (US); Kim C. Tran, Tampa, FL (US); Stine S.H. Olsen, Tampa, FL (US); Charles D. Amsler, JR., Pelham, AL (US); James B. McClintock, Vestavia Hills, AL (US)

(21) Appl. No.: 19/282,474

(22) Filed: Jul. 28, 2025

Related U.S. Application Data
(63) Continuation of application No. PCT/US24/13321, filed on Jan. 29, 2024.
(60) Provisional application No. 63/481,935, filed on Jan. 27, 2023.

(51) Int. Cl. A61K 31/216; A61K 31/122; A61P 31/14

(52) U.S. Cl. CPC A61K 31/216; A61K 31/122; A61P 31/14

(57) ABSTRACT

Novel pharmaceutical compositions and method of treating viral respiratory infections using novel sesterterpenoids is presented. The compounds were found to have antiviral activity against viruses such as respiratory syncytial virus.

### CROSS REFERENCE TO RELATED APPLICATIONS

This application is a continuation of and claims priority to International Application Serial No. PCT/US2024/013321, entitled “Compositions and Methods of Treating Respiratory Syncytial Virus”, filed Jan. 29, 2024, which claims priority to U.S. Provisional Patent Application Ser. No. 63/481,935, entitled “Neosuberitenone, a New Sesterterpenoid Carbon Skeleton, New Suberitenones, and Bioactivity Against Respiratory Syncytial Virus, From the Antarctic Sponge Suberites sp.”, filed Jan. 27, 2023, the contents of each of which are hereby incorporated by reference into this disclosure.

### GOVERNMENT SUPPORT

This invention was made with Government support under Grant No. PLR-1341339, PLR-1341333 and DMR-1644779 awarded by the National Science Foundation. The Government has certain rights in the invention.

### FIELD OF INVENTION

This invention relates to novel compounds and methods of treating respiratory infections. Specifically, the invention provides a novel compounds and methods of treating respiratory syncytial virus (RSV) using synthetic compounds produced from the Suberites species.

### BACKGROUND OF THE INVENTION

With the recent emergence of SARS-COV-2, widespread attention has been drawn to the severity of respiratory illness caused by viral infections. Aside from this and well-known influenza, respiratory syncytial virus (RSV) is another major global pathogen, responsible for bronchiolitis and pneumonia in infants and toddlers under the age of two, as well as severe, sometimes deadly, pneumonia, chronic obstructive pulmonary disease and asthma in elderly adults.¹⁻² Currently there are only two FDA approved drugs for the treatment of RSV, the guanosine analogue ribavirin and the monoclonal antibody palivizumab,³ however both have significant drawbacks. The use of ribavirin is limited to RSV infections in immunocompromised patients owing to its nonspecific activity and toxicity, in conjunction with its relatively high cost,⁴ while palivizumab is only recommended for prophylactic use in high-risk infants and children.⁵ Therefore, there is a current need for new effective and affordable treatments for the widespread RSV pathogen.

Marine sponges collected in the cold waters of Antarctica continue to be a fruitful source of novel bioactive metabolites.⁶⁻⁸ The suberitenones are a class of oxidized sesterterpenes of the ‘suberitane’ carbon skeleton, and have been reported from multiple Antarctic Suberites samples as well as Phorbus areolatus collected in the same waters.⁹⁻¹² More recently, anvilone A and B, two metabolites that also share the same carbon skeleton backbone, were reported from a Phorbus sp. sample collected in the temperate waters of Anvil Island, British Columbia.¹³ These metabolites have displayed a range of bioactivities, with oxaspirosuberitenone demonstrating mild antibacterial activity against MRSA, isosuberitenone B and 19-episuberitenone B showing weak cytotoxicity against a panel of tumor cell lines, and anvilone A turned on HIV gene expression, showcasing the potential of this class of metabolites for biomedical applications.

Given the lack of effective treatments for respiratory infections such as RSV, what is needed are RSV treatments that are efficacious and have little side effects.

### SUMMARY OF INVENTION

Respiratory syncytial virus (RSV) is a highly contagious human pathogen that poses a significant threat to children under the age of two, and there is a current need for new small molecule treatments. The Antarctic sponge Suberites sp. is a known source of sesterterpenes, and following an NMR-guided fractionation procedure was found to produce several previously unreported metabolites. Neosuberitenone (1), with new carbon scaffold herein termed the ‘neosuberitane’ backbone, six suberitenone derivatives (2-7), an ansellane-type terpenoid (8), and a highly degraded sesterterpene (9), as well as previously reported suberitenones A (10) and B (11), were characterized. The structures of all isolated metabolites including absolute configurations are proposed based on NMR, HRESIMS, optical rotation and XRD data. The biological activities of the metabolites were evaluated in a range of infectious disease assays. Suberitenones A, B and F (3) were found to be active against RSV, though, along with other Suberites sp. metabolites, were inactive in bacterial and fungal screens. None of the metabolites were cytotoxic for J774 macrophages or A549 adenocarcinoma cells. The selectivity of suberitenones A, B and F for RSV among other infectious agents is noteworthy.

In an embodiment, a method of treating a viral respiratory infection in a patient in need thereof is presented comprising administering to the patient a therapeutically effective amount of a pharmaceutical composition comprising at least one compound selected from the group consisting of compound 2, compound 3, compound 4, compound 5, compound 6, compound 7, compound 8, compound 9, compound 10, compound 11, and pharmaceutically acceptable salts thereof as depicted in FIG. 1 and a pharmaceutically acceptable carrier wherein the therapeutically effective amount of the composition treats the respiratory infection of the patient. In some embodiments, the pharmaceutical composition further comprises a bioavailability enhancer.

The virus may be from the Paramyxovirdae family. The virus causing the viral respiratory infection may be selected from the group consisting of respiratory syncytial virus (RSV), human metapneumovirus (HMPV), and parainfluenza virus. In some embodiments, the virus is RSV. In some embodiments, the compound is compound 3, compound 10, compound 11, or a pharmaceutically acceptable salt thereof.

In another embodiment, a pharmaceutical composition is presented comprising: at least one synthetic compound selected from the group consisting of compound 1, compound 2, compound 3, compound 4, compound 5, compound 6, compound 7, compound 8, compound 9, and pharmaceutically acceptable salts thereof as depicted in FIG. 1 and a pharmaceutically acceptable carrier. In some embodiments, the at least one compound is compound 3. In some embodiments, the pharmaceutical composition further comprises a bioavailability enhancer.

In a further embodiment, a method of inhibiting viral gene expression in a cell is presented comprising contacting at least one cell with a therapeutically effective amount of at least one compound selected from the group consisting of compound 2, compound 3, compound 4, compound 5, compound 6, compound 7, compound 8, compound 9, compound 10, compound 11, and pharmaceutically acceptable salts thereof as depicted in FIG. 1 wherein the at least one compound inhibits viral gene transcription in the cell.

The viral gene may be from a virus from the Paramyxovirdae family. The virus causing the viral respiratory infection is selected from the group consisting of respiratory syncytial virus (RSV), human metapneumovirus (HMPV), and parainfluenza virus. In some embodiments, the virus is RSV. In some embodiments, the compound may be compound 3, compound 10, compound 11, or a pharmaceutically acceptable salt thereof.

### BRIEF DESCRIPTION OF THE DRAWINGS

For a fuller understanding of the invention, reference should be made to the following detailed description, taken in connection with the accompanying drawings, in which:

FIG. 1 is an image depicting sesterterpene compounds 1-11.

FIG. 2 is a table of ¹³C NMR spectroscopic data of compounds 1-6 and 8.

FIG. 3 is a table of ¹H NMR spectroscopic data of compounds 1-6 and 8.

FIG. 4 is a series of images depicting key 2D NMR correlations establishing the structure of neosuberitenone A (1).

FIG. 5A-B are a series of images depicting the x-ray crystal structure for 1 (left) and 2 (right). Hydrogens omitted for clarity.

FIG. 6 is a table of NMR data for neosuberitenone A (1) (500 (¹H) and 125 (¹³C) MHz, CDCl₃).

FIG. 7 is an image depicting ¹H NMR spectrum (500 MHz, CDCl₃) of 1.

FIG. 8 is an image depicting ¹³C NMR spectrum (125 MHz, CDCl₃) of 1.

FIG. 9 is an image depicting COSY NMR spectrum (500 MHz, CDCl₃) of 1.

FIG. 10 is an image depicting HSQC NMR spectrum (500 MHz, CDCl₃) of 1.

FIG. 11 is an image depicting HMBC NMR spectrum (500 MHz, CDCl₃) of 1.

FIG. 12 is an image depicting NOESY NMR spectrum (600 MHZ, CDCl₃) of 1.

FIG. 13 is an image depicting HRESIMS analysis of 1.

FIG. 14 is an table of crystal data and structure refinement for neosuberitenone A (1).

FIG. 15 is an image depicting an ellipsoid plot of neosuberitenone A (1). Anisotropic displacement parameters were drawn at 50% probability level.

FIG. 16 is a table of NMR data for suberitenone E (2) (500 (¹H) and 125 (¹³C) MHz, CD₃OD).

FIG. 17 is an image depicting ¹H NMR spectrum (500 MHZ, CD₃OD) of 2.

FIG. 18 is an image depicting ¹³C NMR spectrum (150 MHz, CD₃OD) of 2 (formic acid impurity δ_{C} 170.3).

FIG. 19 is an image depicting COSY NMR spectrum (500 MHZ, CD₃OD) of 2.

FIG. 20 is an image depicting HSQC NMR spectrum (500 MHz, CD₃OD) of 2.

FIG. 21 is an image depicting HMBC NMR spectrum (500 MHZ, CD₃OD) of 2.

FIG. 22 is an image depicting NOESY NMR spectrum (500 MHZ, CD₃OD) of 2.

FIG. 23 is an image depicting HRESIMS analysis of 2.

FIG. 24 is an table of the crystal data and structure refinement for suberitenone E (2).

FIG. 25 is an image depicting an ellipsoid plot of suberitenone E (2). Anisotropic displacement parameters were drawn at 50% probability level.

FIG. 26 is a series of images depicting the proposed biogenesis of suberitane and neosuberitane backbone based on compounds isolated in this application.

FIG. 27 is a table of NMR data for suberitenone F (3) (600 (¹H) and 150 (¹³C) MHz, CD₃OD).

FIG. 28 is an image depicting ¹H NMR spectrum (600 MHZ, CD₃OD) of 3.

FIG. 29 is an image depicting ¹³C NMR spectrum (150 MHz, CD₃OD) of 3.

FIG. 30 is an image depicting COSY NMR spectrum (600 MHZ, CD₃OD) of 3.

FIG. 31 is an image depicting HSQC NMR spectrum (600 MHZ, CD₃OD) of 3.

FIG. 32 is an image depicting HMBC NMR spectrum (500 MHz, CD₃OD) of 3.

FIG. 33 is an image depicting NOESY NMR spectrum (600 MHZ, CD₃OD) of 3.

FIG. 34 is an image depicting HRESIMS analysis of 3.

FIG. 35 is a table of NMR data for suberitenone G (4) (500 (¹H) and 150 (¹³C) MHz, CD₃OD).

FIG. 36 is an image depicting ¹H NMR spectrum (500 MHz, CD₃OD) of 4

FIG. 37 is an image depicting ¹³C NMR spectrum (150 MHz, CD₃OD) of 4 (formic acid impurity δ_{C} 170.3).

FIG. 38 is an image depicting COSY NMR spectrum (500 MHZ, CD₃OD) of 4.

FIG. 39 is an image depicting HSQC NMR spectrum (500 MHZ, CD₃OD) of 4.

FIG. 40 is an image depicting HMBC NMR spectrum (500 MHz, CD₃OD) of 4.

FIG. 41 is an image depicting NOESY NMR spectrum (600 MHZ, CD₃OD) of 4.

FIG. 42 is an image depicting HRESIMS analysis of 4.

FIG. 43 is a table of NMR data for suberitenone H (5) (500 (¹H) and 150 (¹³C) MHZ, CD₃OD).

FIG. 44 is an image depicting ¹H NMR spectrum (500 MHZ, CD₃OD) of 5.

FIG. 45 is an image depicting ¹³C NMR spectrum (150 MHz, CD₃OD) of 5.

FIG. 46 is an image depicting COSY NMR spectrum (500 MHZ, CD₃OD) of 5.

FIG. 47 is an image depicting HSQC NMR spectrum (500 MHZ, CD₃OD) of 5.

FIG. 48 is an image depicting HMBC NMR spectrum (500 MHZ, CD₃OD) of 5.

FIG. 49 is an image depicting NOESY NMR spectrum (600 MHZ, CD₃OD) of 5.

FIG. 50 is an image depicting HRESIMS analysis of 5.

FIG. 51 is an image depicting key NOESY correlations establishing the relative stereochemistry of suberitenone H (5).

FIG. 52 is an table of NMR data for suberitenone I (6) (500 (¹H) and 150 (¹³C) MHz, CD₃OD).

FIG. 53 is a table of Assigned NMR data for compound 7 (500 (¹H) and 150 (¹³C) MHz, CD₃OD) using correlations observed when acquiring data on 6.

FIG. 54 is an image depicting ¹H NMR spectrum (600 MHZ, CD₃OD) of 6.

FIG. 55 is an image depicting ¹³C NMR spectrum (150 MHz, CD₃OD) of 6.

FIG. 56 is an image depicting ¹H NMR spectrum (600 MHZ, CD₃OD) of 7.

FIG. 57 is an image depicting COSY NMR spectrum (500 MHZ, CD₃OD) of 6.

FIG. 58 is an image depicting HSQC NMR spectrum (500 MHz, CD₃OD) of 6 (extra signals due to conversion to 7 during acquisition).

FIG. 59 is an image depicting HMBC NMR spectrum (500 MHZ, CD₃OD) of 6 (extra signals due to conversion to 7 during acquisition).

FIG. 60 is an image depicting NOESY NMR spectrum (600 MHZ, CD₃OD) of 6 (extra signals due to conversion to 7 during acquisition).

FIG. 61 is an image depicting HRESIMS analysis of 6.

FIG. 62 is a series of images depicting Chromatogram of C18 HPLC separation of 6 and 7 at 210 nm and their UV/Vis spectra.

FIG. 63 is a table of NMR data for secosuberitenone A (8) (600 (¹H) and 150 (¹³C) MHz, CDCl₃).

FIG. 64 is an image depicting ¹H NMR spectrum (600 MHz, CDCl₃) of 8.

FIG. 65 is an image depicting ¹³C NMR spectrum (125 MHZ, CDCl₃) of 8.

FIG. 66 is an image depicting COSY NMR spectrum (600 MHZ, CDCl₃) of 8.

FIG. 67 is an image depicting HSQC NMR spectrum (600 MHZ, CDCl₃) of 8.

FIG. 68 is an image depicting HMBC NMR spectrum (600 MHZ, CDCl₃) of 8.

FIG. 69 is an image depicting NOESY NMR spectrum (600 MHZ, CDCl₃) of 8.

FIG. 70 is an image depicting HRESIMS analysis of 8.

FIG. 71 is a table of NMR data for norsuberitenone A (9) (500 (¹H) and 125 (¹³C) MHz, CD₃OD).

FIG. 72 is an image depicting ¹H NMR spectrum (500 MHZ, CD₃OD) of 9.

FIG. 73 is an image depicting ¹³C NMR spectrum (150 MHz, CD₃OD) of 9 (formic acid impurity δ_{C} 170.3).

FIG. 74 is an image depicting COSY NMR spectrum (500 MHZ, CD₃OD) of 9.

FIG. 75 is an image depicting HSQC NMR spectrum (500 MHZ, CD₃OD) of 9.

FIG. 76 is an image depicting HMBC NMR spectrum (500 MHZ, CD₃OD) of 9.

FIG. 77 is an image depicting NOESY NMR spectrum (600 MHZ, CD₃OD) of 9.

FIG. 78 is an image depicting HRESIMS analysis of 9.

FIG. 79 is an image depicting RSV antiviral activities of isolated compounds.

FIG. 80 is a table of IC₅₀ values of isolated compounds against RSV.

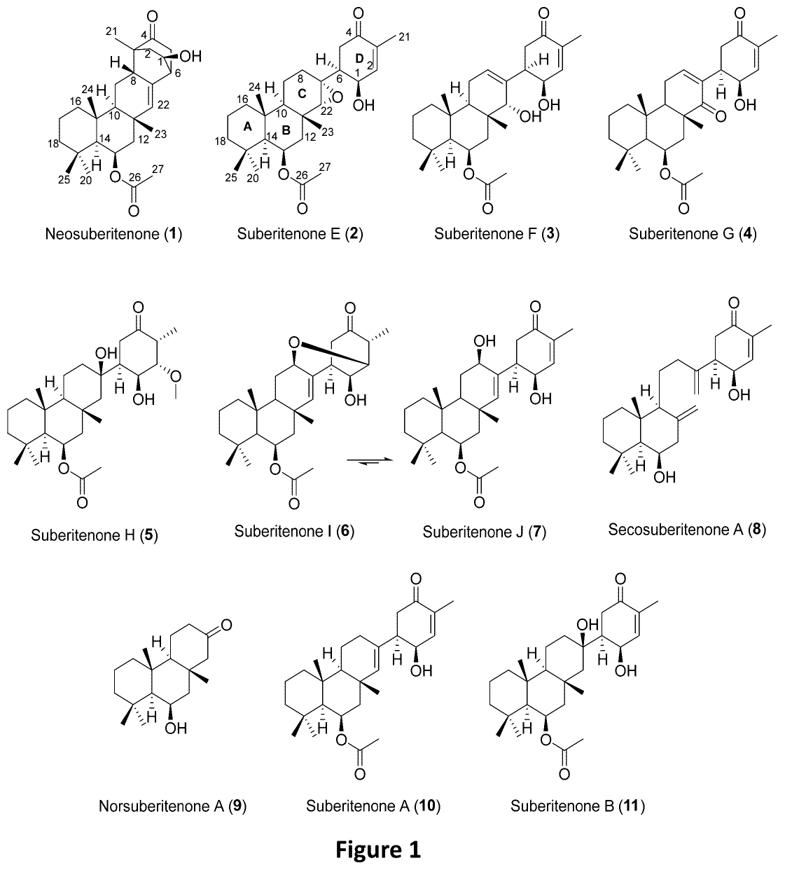

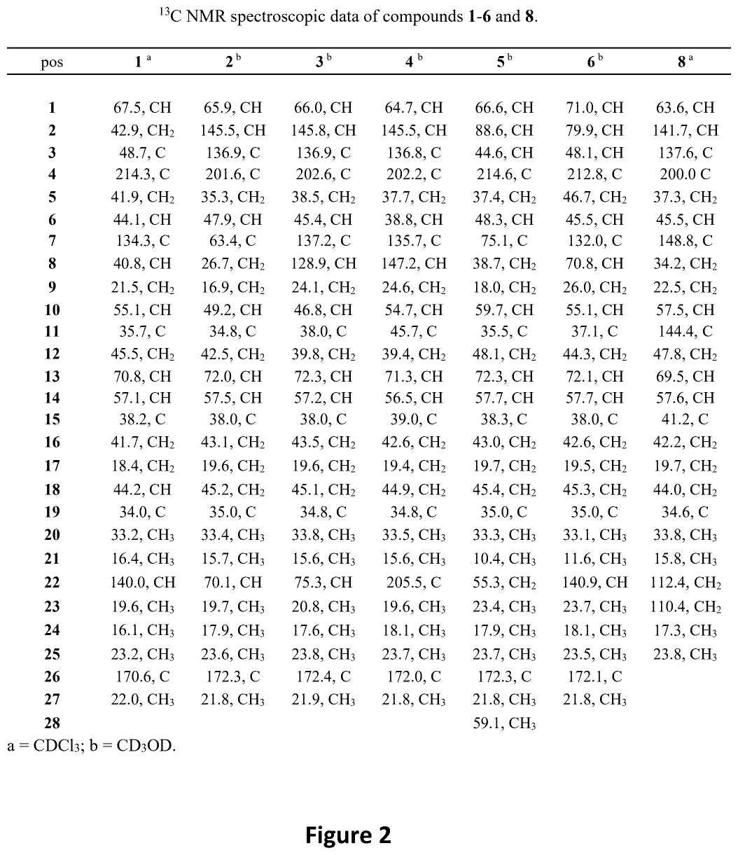

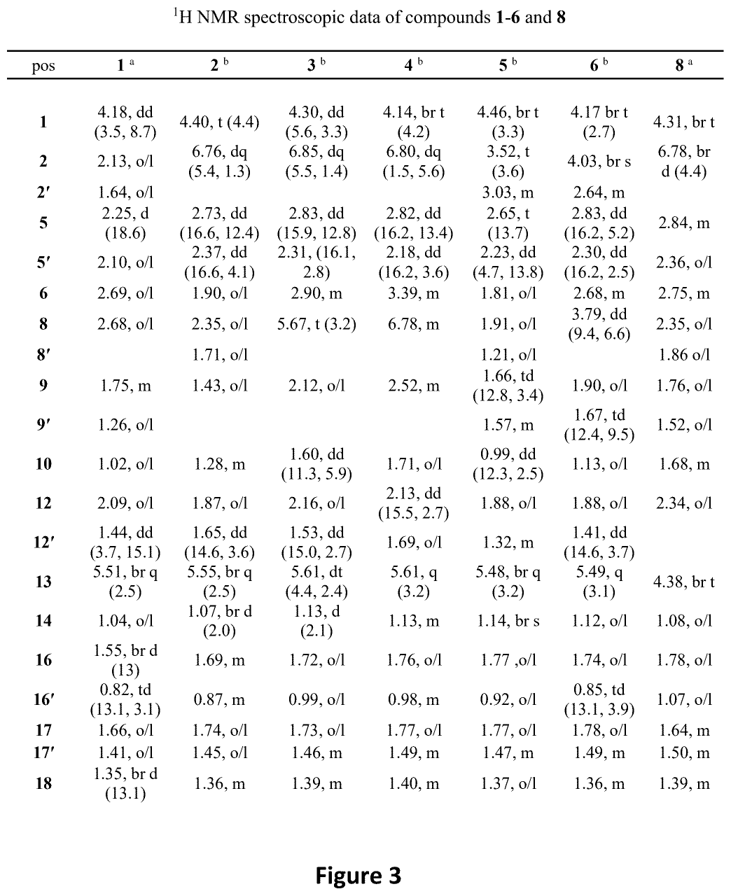

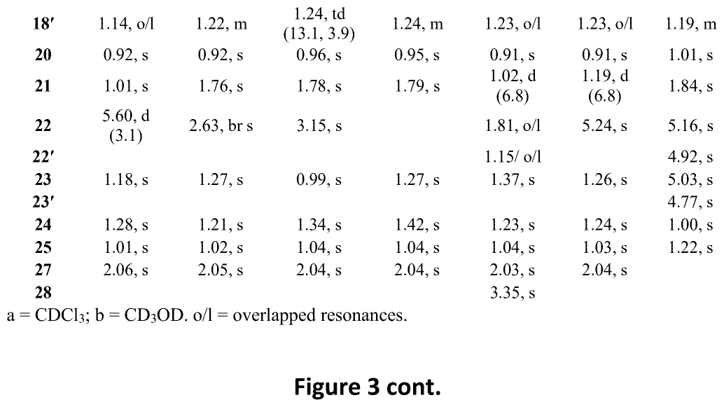

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

As used herein, the singular forms “a,” “an” and “the” include plural referents unless the context clearly dictates otherwise. For example, “a nanoparticle” includes “nanoparticles” or “plurality of nanoparticles”.

As used in this specification and the appended claims, the term “or” is generally employed in its sense including “and/or” unless the context clearly dictates otherwise.

All numerical designations, such as pH, temperature, time, concentration, and molecular weight, including ranges, are approximations which are varied up or down by increments of 1.0, 0.1, 0.01 or 0.001 as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, “about” means approximately or nearly and in the context of a numerical value or range set forth means ±10% of the numerical.

As used herein “patient” is used to describe a mammal, preferably a human, to whom treatment is administered, including prophylactic treatment with the compositions of the present invention. Non-limiting examples of mammals include humans, rodents, aquatic mammals, domestic animals such as dogs and cats, farm animals such as sheep, pigs, cows and horses. “Patient” and “subject” are used interchangeably herein.

“Administering” or “administration” as used herein refers to the process by which the compositions of the present invention are delivered to the patient. The compositions may be administered in various ways, including but not limited to, orally, nasally, and parenterally.

“Parenteral administration” as used herein refers to modes of administration other than enteral and topical administration, usually by injection, and includes, but is not limited to, intravenous, intramuscular, intraarterial, intrathecal, intracapsular, intraorbital, intracardiac, intradermal, intraperitoneal, transtracheal, subcutaneous, subcuticular, intra-articular, subcapsular, intrathecal, intraventricular, intracisternal, intranigral, subarachnoid, intraspinal, and intrasternal injection and infusion. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A “therapeutic agent” as used herein refers to a substance, composition, compound, chemical, component or extract that has measurable specified or selective physiological activity when administered to an individual in a therapeutically effective amount. In some embodiments, the therapeutic agent may be a an antiviral composition. Examples of therapeutic agents as used in the present invention include, but are not limited to, small molecules. At least one therapeutic agent is used in the compositions of the present invention, however in some embodiments, multiple therapeutic agents are used. In some embodiments, the novel compounds and compositions described herein may be combined with another antiviral composition that targets a different area of the virus. In some embodiments, one or more therapeutic agents may be encapsulated within a nanoparticle.

A “therapeutically effective amount” as used herein is defined as concentrations or amounts of components which are sufficient to effect beneficial or desired clinical results, including, but not limited to, any one or more of treating symptoms of viral respiratory infection or preventing viral infection, such as respiratory infections caused by virus from the Paramyxovirdae family, genus Pneumoviridae, and, particularly RSV infection. Compositions of the present invention can be used to effect a favorable change in the condition whether that change is an improvement, such as stopping, reversing, or reducing RSV infection, or a complete elimination of symptoms due to RSV infection. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration. The dose may be adjusted according to response.

The dosing of compounds and compositions to obtain a therapeutic or prophylactic effect is determined by the circumstances of the patient, as is known in the art. The dosing of a patient herein may be accomplished through individual or unit doses of the compounds or compositions herein or by a combined or prepackaged or pre-formulated dose of a compounds or compositions.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current invention according to the foregoing considerations.

In instances where human dosages for compounds have been established for at least some condition, those same dosages may be used, or dosages that are between about 0.1% and 500%, more preferably, between about 25% and 250% of the established human dosage. Where no human dosage is established, as will be the case for newly-discovered pharmaceutical compositions, a suitable human dosage can be inferred from ED 50 or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequently. Administration can also be continuous and adjusted to maintaining a level of the compound within any desired and specified range.

The administration of the composition can be extended over an extended period of time, such as from about a week or shorter up to about a year or longer. For example, the dosing regimen can be extended over a period of any of about 2, 3, 4, 5, 6, 7, 8, 9, 10, 11, and 12 months. In some embodiments, there is no break in the dosing schedule. In some embodiments, the interval between each administration is no more than about a week.

The compounds used in the present invention may be administered individually, or in combination with or concurrently with one or more other compounds used against viruses, including pneumovirus such as RSV. Additionally, compounds used in the present invention may be administered in combination with or concurrently with other therapeutics for RSV or other respiratory viruses. In combination therapy, the additional agents can be administered in amounts that have been shown to be effective for those additional agents. Such amounts are known in the art; alternatively, they can be derived from viral load or replication studies. Alternatively, the amount used can be less than the effective monotherapy amount for such additional agents. For example, the amount used could be between 90% and 5% of such amount, e.g., 90%, 80%, 70%, 60%, 50%, 40%, 30%, 20%, 10%, or 5%, or intermediate values between those points.

“Prevention” or “preventing” or “prophylactic treatment” as used herein refers to any of: halting the effects of pneumovirus infection, reducing the effects of pneumovirus infection, reducing the incidence of pneumovirus infection, reducing the development of pneumovirus infection, delaying the onset of symptoms of pneumovirus infection, increasing the time to onset of symptoms of pneumovirus infection, and reducing the risk of development of pneumovirus infection. In some embodiments, the pneumovirus infection is RSV.

“Treatment” or “treating” as used herein refers to any of the alleviation, amelioration, elimination and/or stabilization of a symptom, as well as delay in progression of a symptom of a particular disorder. For example, “treatment” of pneumovirus infection may include any one or more of the following: amelioration and/or elimination of one or more symptoms associated with pneumovirus infection, reduction of one or more symptoms of pneumovirus infection, stabilization of symptoms of pneumovirus infection, and delay in progression of one or more symptoms of pneumovirus infection. In some embodiments, the pneumovirus infection is RSV.

“Infection” as used herein refers to the invasion of one or more microorganisms such as bacteria, viruses, fungi, yeast, or parasites in the body of a patient in which they are not normally present. In certain embodiments, the infection is from a respiratory virus such as a respiratory syncytial virus (RSV), human metapneumovirus (HMPV), parainfluenza virus, influenza virus or coronavirus. In some embodiments, the respiratory virus is from the Paramyxovirdae family. In some embodiments, the virus is from the Pneumoviridae genus which includes pneumoviruses RSV and HMPV. In some embodiments where the virus is human RSV, human respiratory syncytial virus subgroups A1, A2, B1, and B2 are contemplated. Other RSV are contemplated, such as bovine respiratory syncytial virus and murine pneumonia virus, as well as future pneumoviruses. HMPV subgroups A1, A2, B1, and B2 are contemplated as well as avian metapneumovirus and other future pneumoviruses. In some embodiments, the virus is from the Paramyxovirus genus such as the parainfluenza virus. Other paramyxoviruses are contemplated including other future paramyxoviruses.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W Easton Pennsylvania, Mack Publishing Company, 19^{th} ed.) describes formulations which can be used in connection with the subject invention.

For ease of administration, the subject compounds may be formulated into various pharmaceutical forms. As appropriate compositions there may be cited all compositions usually employed for systemically or topically administering drugs. To prepare the pharmaceutical compositions of this invention, at least one of the sesterterpene compounds, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for administration nasally, orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their case in administration, tablets and capsules often represent the most advantageous oral dosage unit form, in which case solid pharmaceutical carriers are obviously employed. For parenteral compositions, the carrier may comprise sterile water, with other ingredients, for example, to aid solubility, included. Injectable solutions, for example, may be prepared in which the carrier comprises saline solution, glucose solution or a mixture of saline and glucose solution. In some embodiments, the pharmaceutically acceptable carrier is a carrier other than water or saline. In some embodiments, the pharmaceutically acceptable carrier is part water or saline mixed with another carrier such as a synthetic carrier.

A “bioavailability enhancer” as used herein is an agent or combination of agents that enhance the rate and/or extent of absorption of a compound, such as a sesterterpene compound as described herein, that reaches the systemic circulation and is available at the site of action. A bioavailability enhancer may also improve tissue distribution and targeting of the compound. Examples of bioavailability enhancers include, but are not limited to, liposomes, vitamin E, TPGS (d-α-tocopheryl polyethylene glycol 1000 succinate); acetylated monoglycerides; mono-, di-, and triglyceride esters of medium-chain (6-12 carbon atoms in length) and long-chain (more than 12 carbon atoms in length) fatty acids; esters of fatty acids and glycols or glycerol; esters of mixed fatty acids and glycols or glycerol; diesters of propylene glycol having from about 7 to about 55 carbon atoms; propylene glycol esters of capric and caprylic acids; citric acid, malic acid, ascorbic acid, fumarie acid, caproic acid, caprylic acid, cholic acid, glycocholic acid, sodium cholate, sodium lauryl sulfate, palmitoyl carnitin, cyclosporin A, polyoxyethylene/polyoxypropylene copolymers and other soluble polymers, solid lipid nanoparticles, and mixtures thereof. Soluble bioavailability-enhancing polymers to which compounds may be coupled to as targetable carriers include polyvinylpyrrolidone, pyran copolymer, polyhydroxylpropylmethacrylamide-phenoi, polyhydroxyethylasparta-midephenol, or polyethyleneoxide-polylysine substituted with palmitoyl residues. Furthermore, the compounds or drugs may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The term “synthetic” or “synthetically derived” as used herein refers to a product produced artificially by human hand by chemical synthesis. In some cases, “synthetic” refers to the manufacture of a product which mimics a natural product. Both natural and synthetic products can be used to manufacture the pharmaceutical compositions described herein.

Unless stated to the contrary, a formula with chemical bonds shown only as solid lines and not as wedges or dashed lines contemplates each possible isomer, e.g. each enantiomer and diastereomer, and a mixture of isomers, such as racemic or scalemic mixture. Compounds described herein can contain one or more asymmetric centers and, thus potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included.

Unless otherwise stated, structures depicted herein are also meant to include all isomeric (e.g., enantiomeric, diastereomeric, optical, and geometric (or conformational)) forms of the structure or a form thereof (including salts, solvates, esters, and prodrugs and transformed prodrugs thereof); for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms.

The compounds described herein or a form thereof described herein may include one or more chiral centers, and as such may exist as racemic mixtures (R/S) or as substantially pure enantiomers and diastereomers. The compounds may also exist as substantially pure (R) or(S) enantiomers (when one chiral center is present). In one embodiment, the compounds described herein or a form thereof described herein are(S) isomers and may exist as enantiomerically pure compositions substantially comprising only the(S) isomer. In another embodiment, the compounds described herein or a form thereof described herein are (R) isomers and may exist as enantiomerically pure compositions substantially comprising only the (R) isomer. As one of skill in the art will recognize, when more than one chiral center is present, the compounds described herein or a form thereof described herein may also exist as a (R,R), (R,S), (S,R) or (S,S) isomer, as defined by IUPAC Nomenclature Recommendations.

As used herein, the term “substantially pure” refers to compounds described herein or a form thereof consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

As used herein, the term “racemate” refers to any mixture of isometric forms that are not “enantiomerically pure”, including mixtures such as, without limitation, in a ratio of about 50/50, about 60/40, about 70/30, or about 80/20, about 85/15 or about 90/10.

All stereoisomers (for example, geometric isomers, optical isomers and the like) of the present compounds described herein or a form thereof (including salts, solvates, esters and prodrugs and transformed prodrugs thereof), which may exist due to asymmetric carbons on various substituents, including enantiomeric forms (which may exist even in the absence of asymmetric carbons), rotameric forms, atropisomers, diastereomeric and regioisomeric forms, are contemplated within the scope of the description herein. Individual stereoisomers of the compounds described herein or a form thereof described herein may, for example, be substantially free of other isomers, or may be present in a racemic mixture, as described supra.

Diastereomeric mixtures can be separated into their individual diastereomers on the basis of their physical chemical differences by methods well known to those skilled in the art, such as, for example, by chromatography and/or fractional crystallization. Enantiomers can be separated by use of a chiral HPLC column or other chromatographic methods known to those skilled in the art.

Enantiomers can also be separated by converting the enantiomeric mixture into a diastereomeric mixture by reaction with an appropriate optically active compound (e.g., chiral auxiliary such as a chiral alcohol or Masher's acid chloride), separating the diastereomers and converting (e.g., hydrolyzing) the individual diastereomers to the corresponding pure enantiomers.

The term “isotopologue” refers to isotopically-enriched compounds described herein or a form thereof which are identical to those recited herein, but for the fact that one or more atoms are replaced by an atom having an atomic mass or mass number different from the atomic mass or mass number usually found in nature.

One or more compounds described herein or a form thereof described herein may exist in unsolvated as well as solvated forms with pharmaceutically acceptable solvents such as water, ethanol, and the like, and the description herein is intended to embrace both solvated and unsolvated forms

As used herein, the term “solvate” means a physical association of a compound described herein or a form thereof described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates.

The term “compound” as used herein refers to a chemical formulation, either organic or inorganic, that induces a desired pharmacological and/or physiological effect on a subject when administered in a therapeutically effective amount. “Compound” is used interchangeably herein with “drug” and “therapeutic agent”. When the compound name disclosed herein conflicts with the structure depicted, the structure shown will supersede the use of the name to define the compound intended.

The compounds described herein or a form thereof can form salts, which are intended to be included within the scope of this description. Reference to a compound or a form thereof herein is understood to include reference to salts thereof, unless otherwise indicated. The term “salt(s)”, as employed herein, denotes acidic salts formed with inorganic and/or organic acids, as well as basic salts formed with inorganic and/or organic bases.

The term “pharmaceutically acceptable salt(s)”, as used herein, means those salts of compounds disclosed or a form thereof described herein that are safe and effective (i. e., non-toxic, physiologically acceptable) for use in mammals and that possess biological activity, although other salts are also useful. All such acid salts and base salts are intended to be included within the scope of pharmaceutically acceptable salts as described herein.

In addition, all such acid and base salts are considered equivalent to the free forms of the corresponding compounds for purposes of this description.

The use of the terms “salt,” “solvate,” “ester,” “prodrug” and the like, is intended to apply equally to the salt, solvate, ester and prodrug of enantiomers, stereoisomers, rotamers, tautomers, positional isomers, racemates, isotopologues or prodrugs of the instant compounds.

As used herein, the term “substituent” means positional variables on the atoms of a core molecule that are attached at a designated atom position, replacing one or more hydrogen atoms on the designated atom, provided that the atom of attachment does not exceed the available valence or shared valence, such that the substitution results in a stable compound. Accordingly, combinations of substituents and/or variables are permissible only if such combinations result in stable compounds. Any carbon atom as well as heteroatom with a valence level that appears to be unsatisfied as described or shown herein is assumed to have a sufficient number of hydrogen atom(s) to satisfy the valences described or shown.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

As part of the ongoing investigation into the chemistry of Antarctic marine organisms, a Suberites sp. sample collected in 2018 was subjected to a ¹H NMR guided purification procedure using reversed-phase HPLC. The previously reported compounds suberitenone A (10) and B (11) were both isolated in large quantities from the least polar fractions, while the ¹H NMR spectra of earlier eluting fractions showed a number of interesting resonances warranting further analysis. This led to the isolation of nine previously unreported sesterterpene compounds, 1 to 9, of which 1 was made up of a previously unreported carbon skeleton and 6 existed as an interconverting equilibrium between two structures 6 and 7.

The following non-limiting examples illustrate exemplary systems and components thereof in accordance with various embodiments of the disclosure. The examples are merely illustrative and are not intended to limit the disclosure in any way.

### Example 1—Isolation of Sesterterpene Compounds

### Neosuberitenone A (1)

Neosuberitenone A (1) was isolated as white crystals, with a molecular formula C₂₇H₄₀O₄ (eight double bond equivalents) established by analysis of the (−)-HRESIMS formate adduction at m/z 473.2915. The 1D ¹³C NMR spectrum (125 MHz, CDCl₃) gave signals for all 27 carbons and in conjunction with the multiplicity-edited HSQC data, suggested 1 contained a ketone, a carboxylate, four sp³ and one sp² quaternary carbons, seven methine carbons of which one is olefinic and two are oxygen bearing, seven methylenes and six methyl groups. This data accounts for three degrees of unsaturation, therefore 1 must also contain five rings. The ¹H NMR spectrum showed signals of two oxygenated methines at δ_{H} 4.18 (dd, J=3.5, 8.7 Hz) and δ_{H} 5.51 (br q, J=3.9 Hz) and an olefin at δ_{H} 5.60 (d, J=3.1 Hz), and it was also observed that the five aliphatic and acetate methyl groups were all singlets, while the remainder of the 1D ¹H NMR spectrum was complicated by overlapping aliphatic signals.

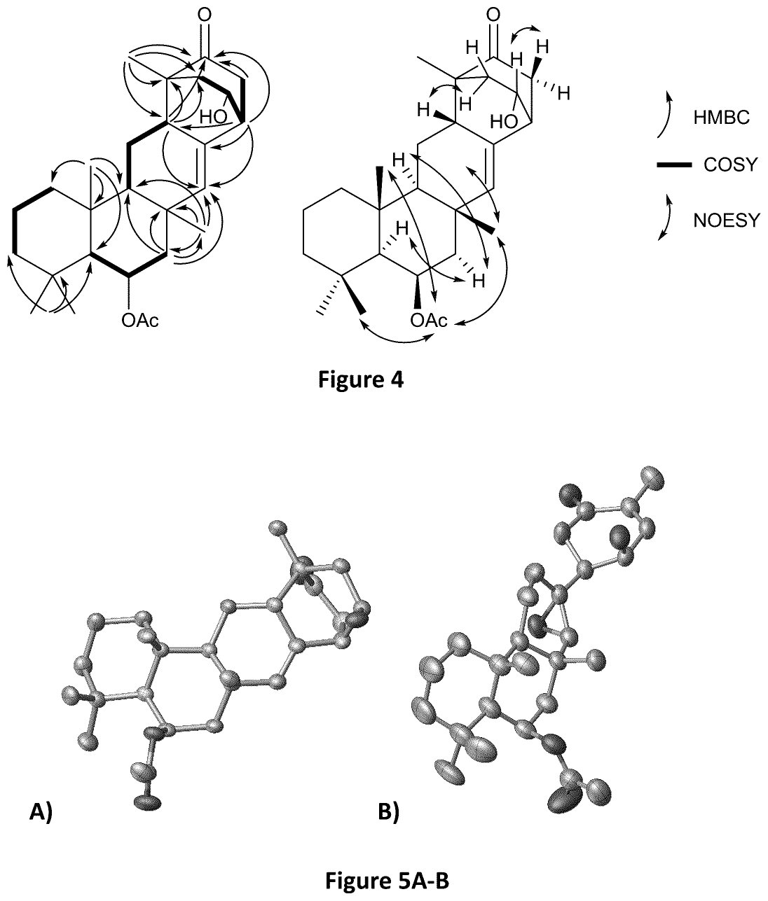

These data, along with correlations in the COSY and HMBC spectra, suggested the structure of neosuberitenone A (1) to be closely related to those of the suberitane class of sesterterpenoids (FIG. 4). The first spin system was deduced from COSY correlations from H₂-16 (δ_{H} 0.82 and 1.55) to H₂-17 (δ_{H} 1.41 and 1.66) and then H₂-18 (δ_{H} 1.14 and 1.35), which comprise the backbone of ring A. The ring assignment was completed using HMBC correlations from the geminal dimethyl H₃-20 and H₃-25 (δ_{H} 0.92 and 1.01 respectively) to C-14, C-18, and C-19 (δ_{C} 57.1, 44.2 and 34.0 respectively), in conjunction with methyl H₃-24 (δ_{H} 1.28) to C-10, C-15, C-16 (δ_{C} 55.1, 38.2 and 41.7) and C-14. Next, the cycle of ring B was assigned using COSY correlations between H-14 (δ_{H} 1.04) and oxygenated H-13 (δ_{H} 5.51) and then onto methylene H₂-12 (δ_{H} 2.09 and 1.44), and HMBC correlations from H₂-12 to C-11, C-22, C-23 (δ_{C} 35.7, 140.0 and 19.6 respectively) and C-10. Methyl group H₃-23 (δ_{H} 1.18) showed HMBC correlations to C-10, C-11, C-12, and C-22 so was therefore placed on quaternary C-12. Ring C however, showed the differences from the suberitane class, lacking the C-8 methylene which was instead replaced by a methine (δ_{H} 2.68) that was assigned using COSY correlations to H₂-9 (δ_{H} 1.75 and 1.26) and H-10 (δ_{H} 1.02) and HMBC correlation to olefin C-22.

The remainder of neosuberitenone A (1) consisted of one major spin system between H₂-2 (δ_{H} 2.13 and 1.64), oxygenated H-1 (δ_{H} 4.18), H-6 (δ_{H} 2.69) and H₂-5 (δ_{H} 2.25 and 2.10) deduced from COSY correlations. The HMBC spectrum showed correlations from H-8 to C-2, C-3, and C-4 (δ_{C} 42.9, 48.7 and 214.3 respectively) which connected the two units, while the methyl H₃-21 (δ_{H} 1.01) was placed at C-3 as it showed HMBC correlations to C-2, C-3, C-4, and C-8. This spin system was reasoned to be cyclized between C-4 and C-5 (δ_{C} 41.9) as both H₂-5 and H-6 showed HMBC correlations to ketone C-4. HMBC correlations from H-6 were able to finalize the ring structure of 1, with correlations to C-7, C-8 (δ_{C} 134.3 and 40.8 respectively) and C-22 providing evidence for a bond between C-6 and C-7. The planar structure of 1 was completed by placing the acetyl group on the oxygen of C-13, based on correlations in the NOESY spectrum between H₃-27 (2.06) and H₃-23, H₃-24 and H₃-25 that established the acetyl group was not on the C-1 oxygen.

The stereochemistry of neosuberitenone A (1) was deduced by correlations in the NOESY spectrum. The similar elements of 1 and suberitenone A (9) were determined to have the same stereochemistry, with methyl groups H₃-23, H₃-24 and H₃-25 all deduced to be cofacial with acetyl group H₃-27 by NOESY correlations, while correlations between H-12′ to both H-10 and H-14 also placed these protons on the opposite face of the molecule. A correlation between H₃-23 to H-8 set the orientation of this center relative to the methyl groups previous deduced, and the correlation from H-8 and H-2′ also suggested these two protons to be syn. As C-3 and C-6 are joined by a two carbon bridge, this also set the stereochemistry at C-6 as the bridging carbons must be on the same side of the molecule. Finally, correlations between H-1 and H-5 suggested the H-1 proton to be oriented toward C-5 and thus 1 was deemed to have the 1R*,3R*,6R*,8S*,10S*,11S*,13R*,14S*,15R* configuration.

This spectroscopic data analysis was confirmed by single-crystal X-ray diffraction (XRD) studies, with neosuberitenone A (1) forming suitable crystals from 9:1 ACN:H₂O. The absolute configuration of 1 was determined to be 1R,3R,6R,8S,10S,11S,13R,14S,15R (FIG. 5A), similar to that deduced by Shin and coworkers on suberitenone A (9) and B (10) using modified Mosher's method and CD analysis.⁹ This terpenoid carbon backbone of 1 is an unprecedented class to date, and is termed the ‘neosuberitane’ backbone.

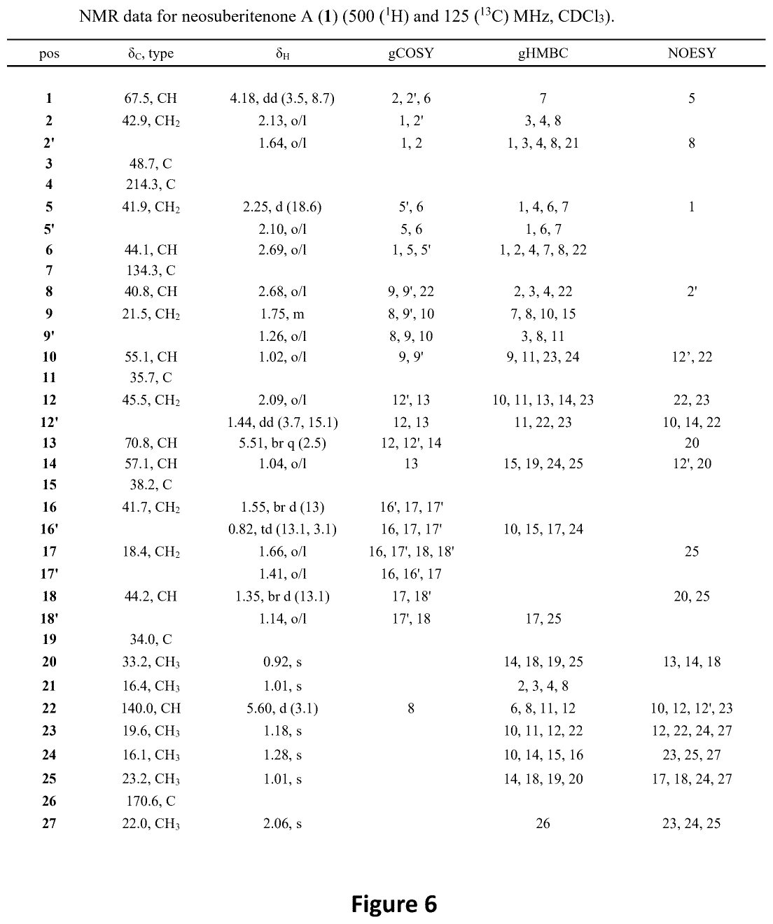

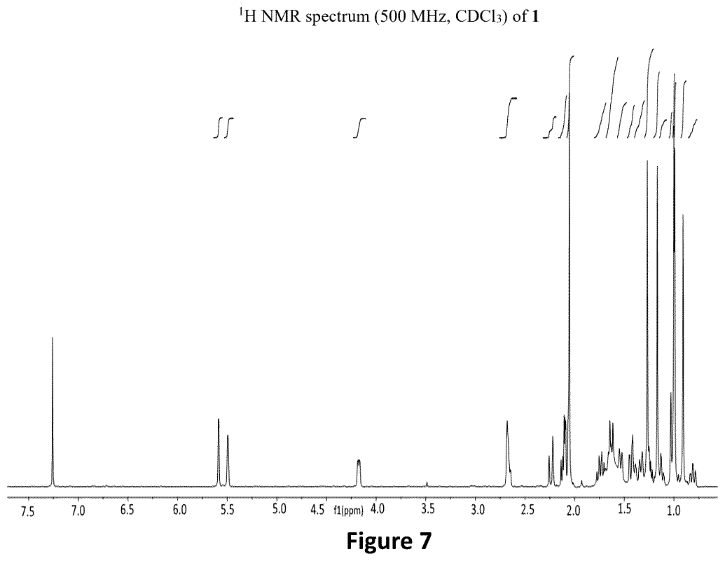

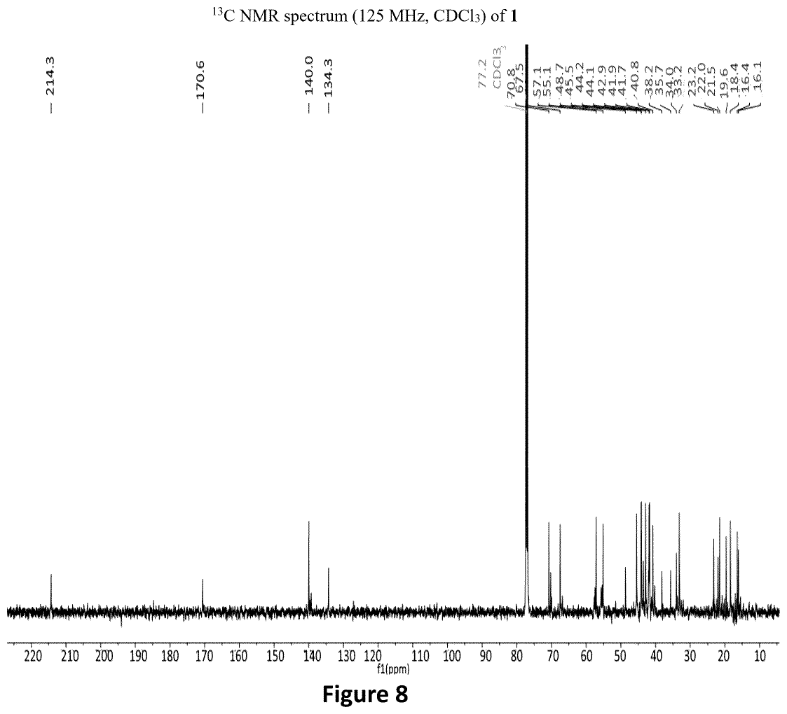

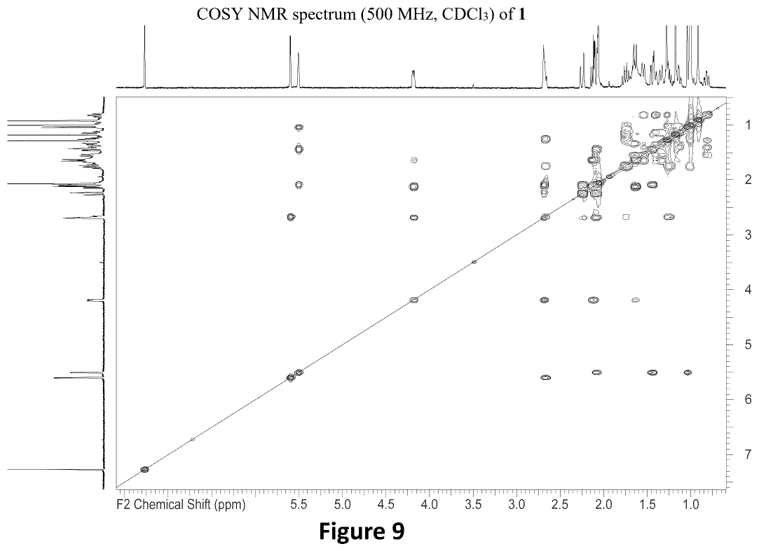

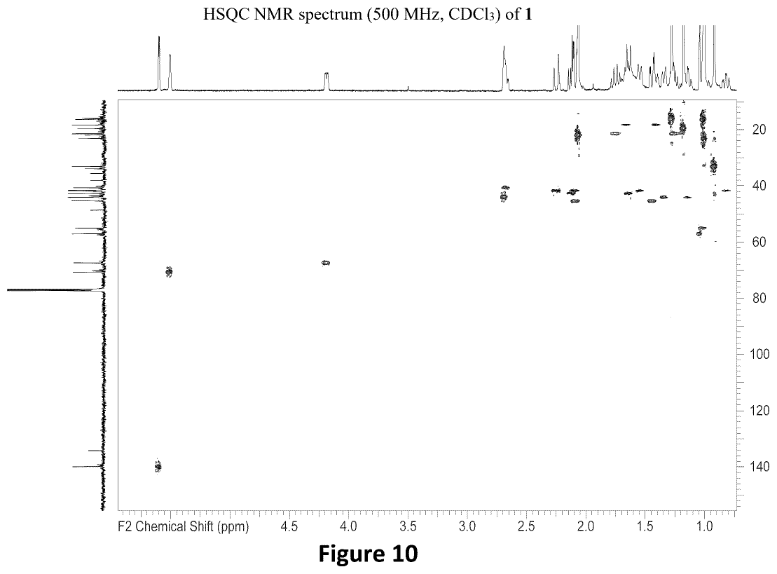

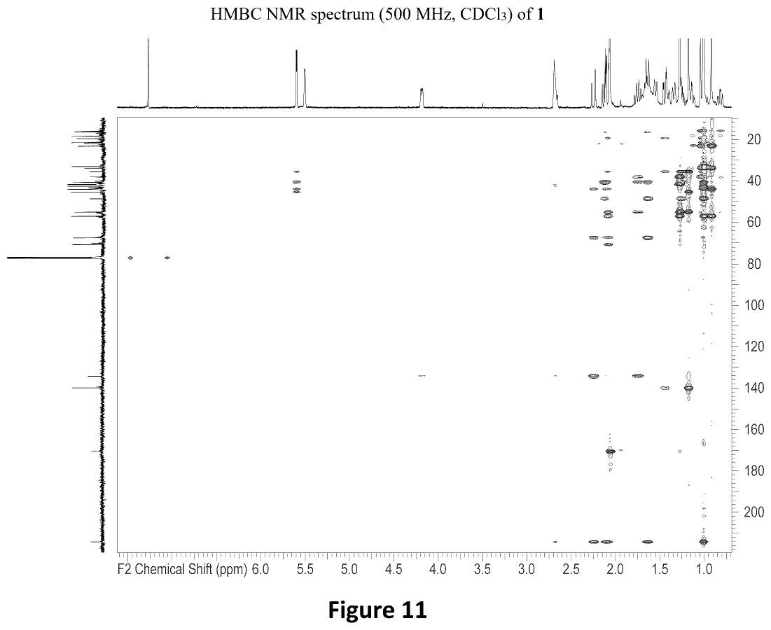

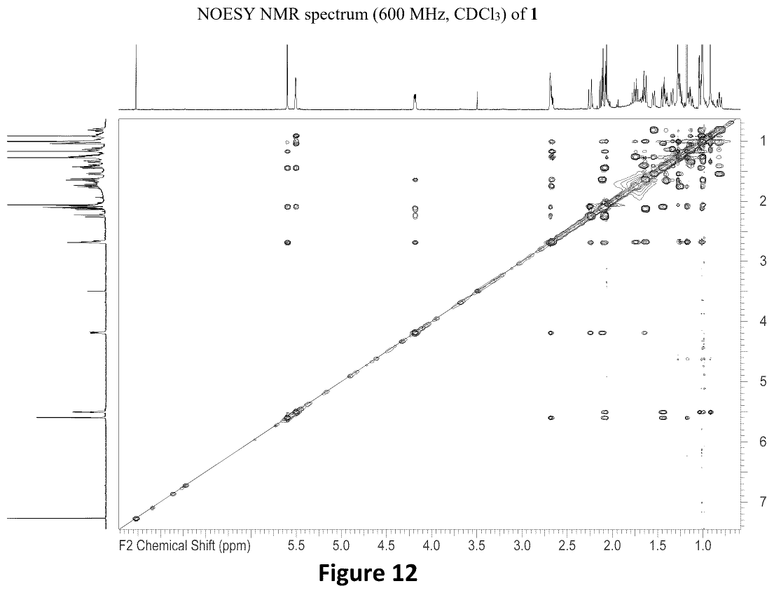

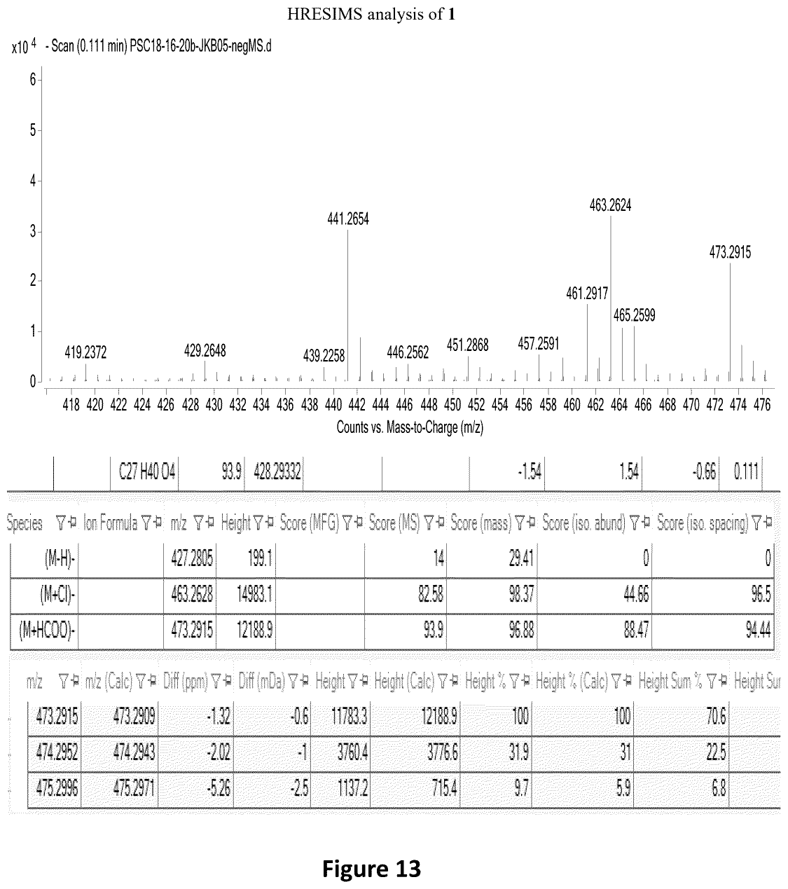

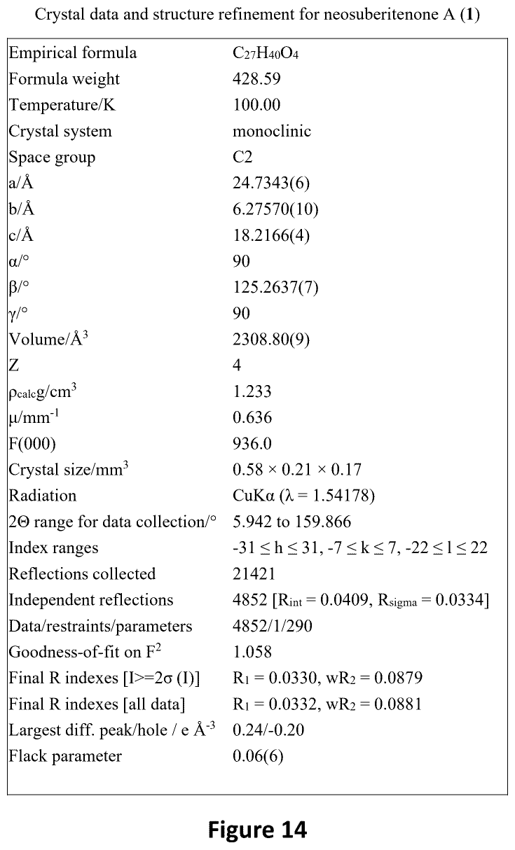

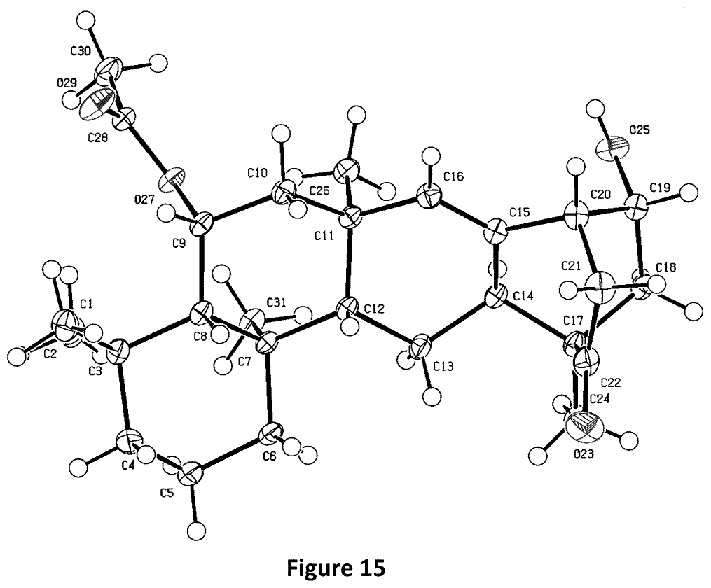

A summary of NMR data for neosuberitenone A (1) is shown in the table of FIG. 6 with NMT spectra and MS data shown in FIGS. 7-13. The crystal data for neosuberitenone A (1) is shown in the table of FIG. 14. The ellipsoid plot of neosuberitenone A (1) is shown in FIG. 15.

### Suberitenone E (2)

Suberitenone E (2) was also isolated as white crystals, with a molecular formula C₂₇H₄₀O₅ established by analysis of the formate adduct ion at m/z 489.2865 in the (−)-HRESIMS data. Analysis of the 1D and 2D NMR data suggested 2 to be structurally related to suberitenone A, with major differences present in ring C, notably the absence of the olefin and introduction of a new singlet for H-22 (δ_{H} 2.63; δ_{C} 70.1). Comparison of the molecular formulas show 2 to have one extra oxygen atom, all of which points to the presence of an epoxide across the C-7/C-22 bond. Correlations in the NOESY spectrum along with coupling constant analysis were used to deduce that all of the stereocenters shared with suberitenone A (9) were of the same orientation, with a correlation between epoxide H-22 and H₃-23 (δ_{H} 1.27) suggesting a syn relationship and therefore establishing the configuration of both C-7 and C-22. Single-crystal XRD confirmed this interpretation and established the absolute configuration as 1R,6S,7R,10R,11R,13R,14S,15R,22R as in FIG. 5B.

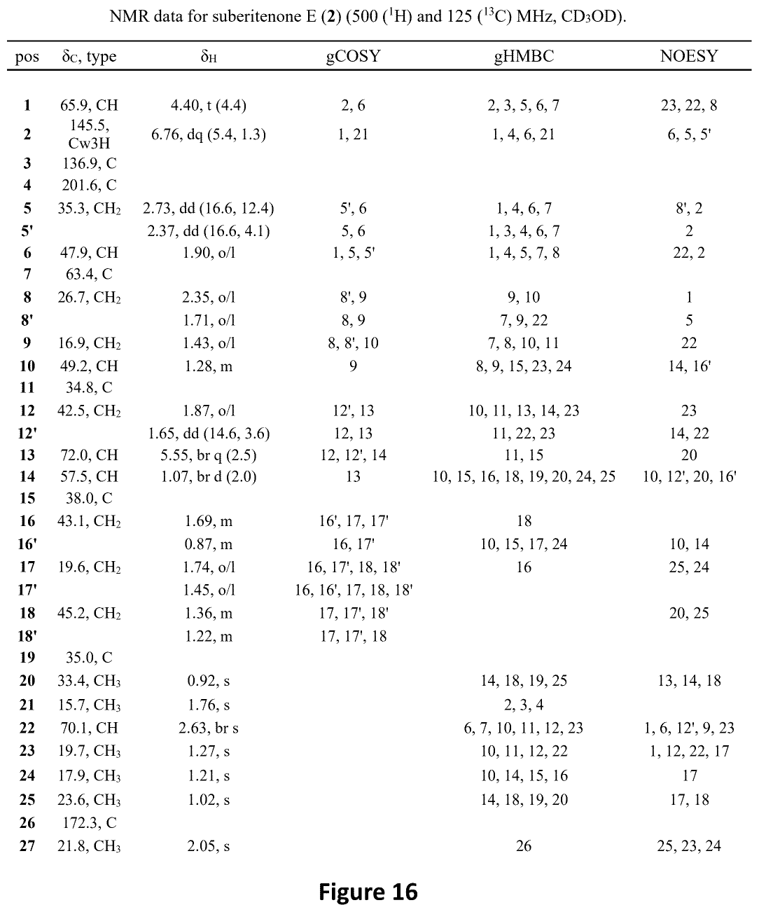

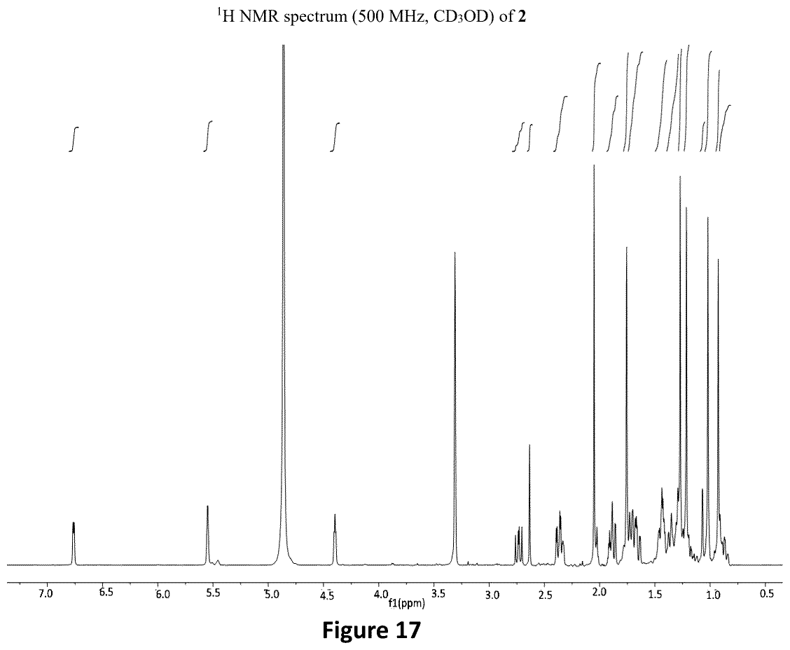

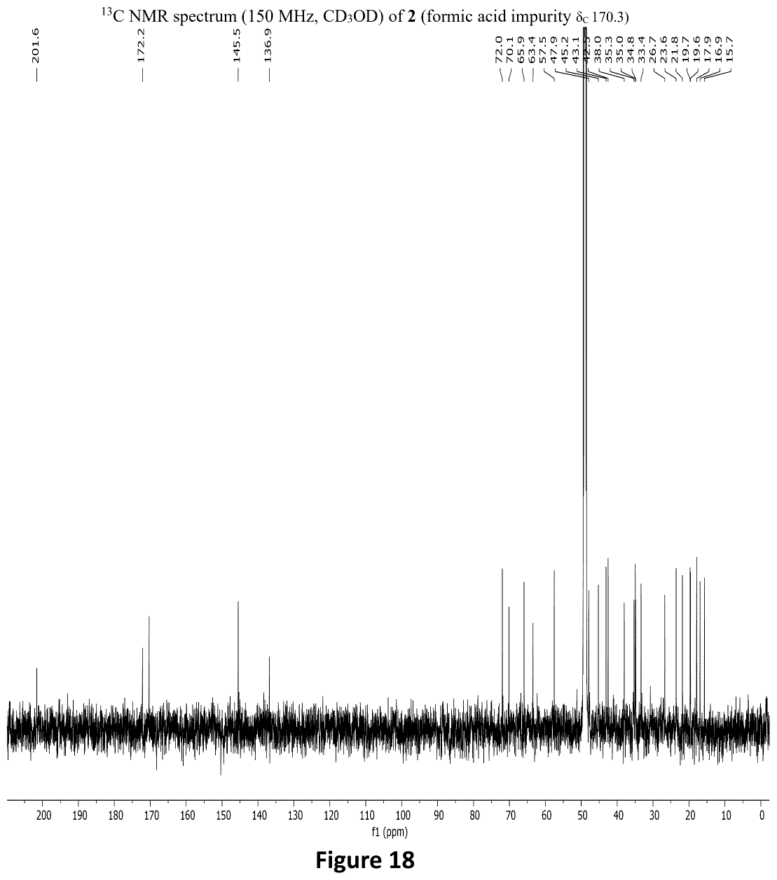

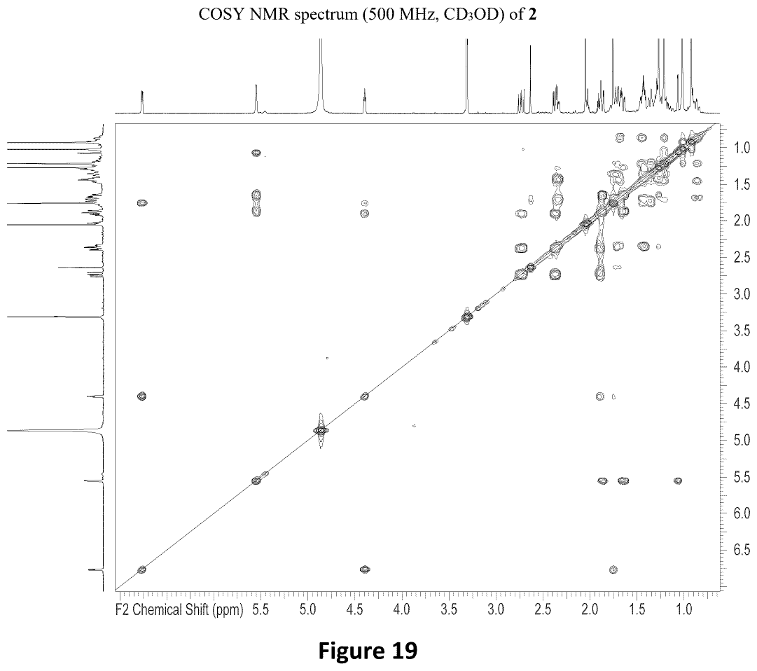

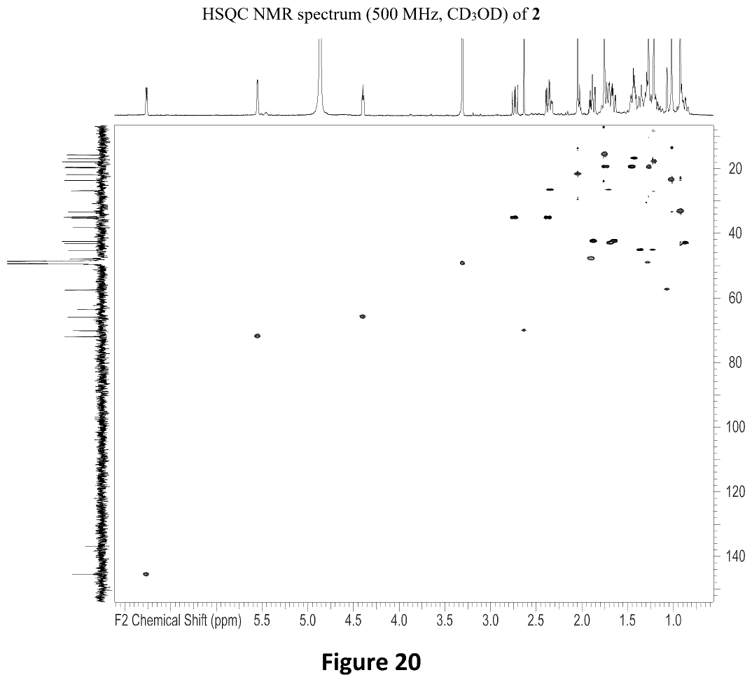

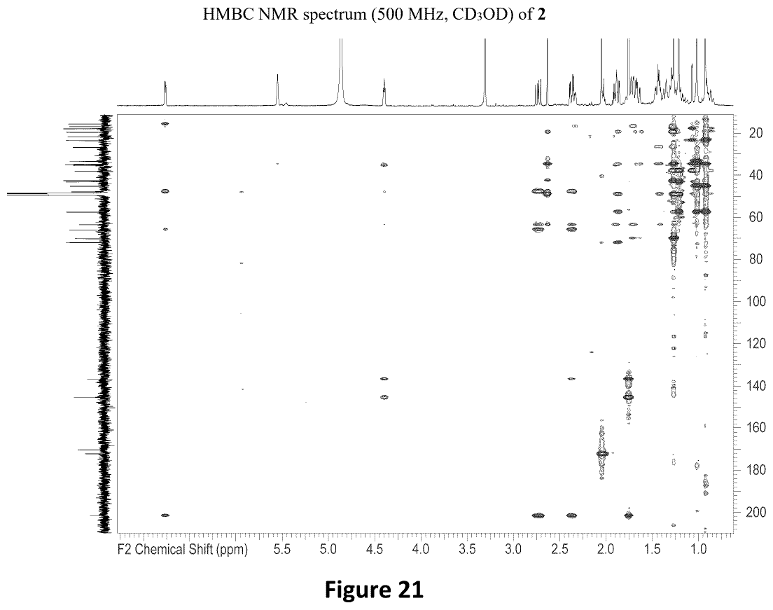

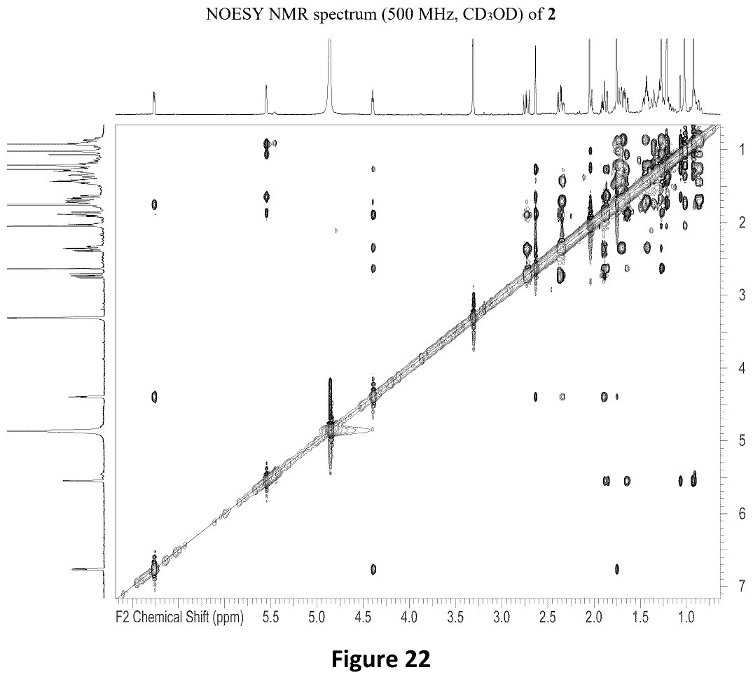

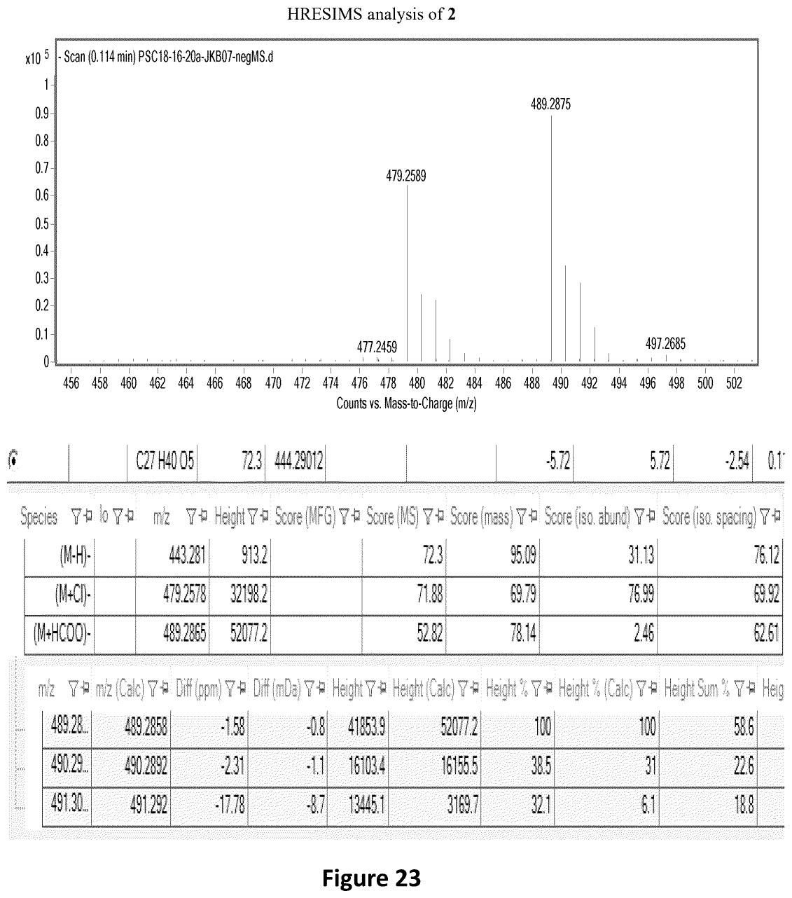

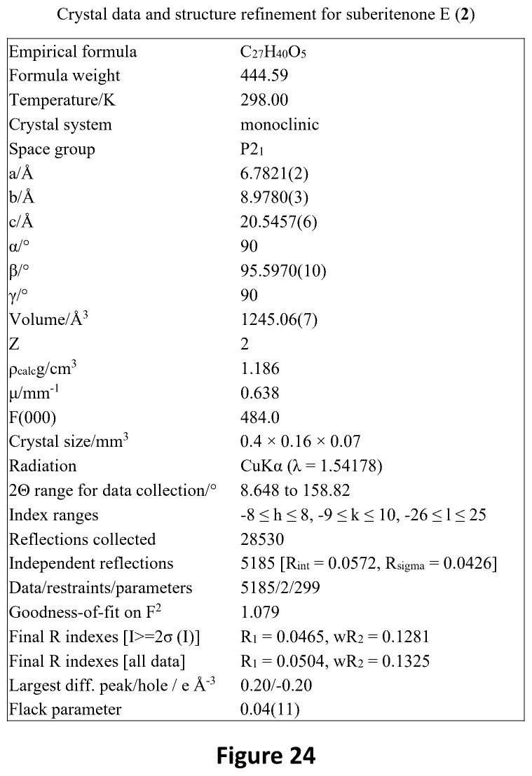

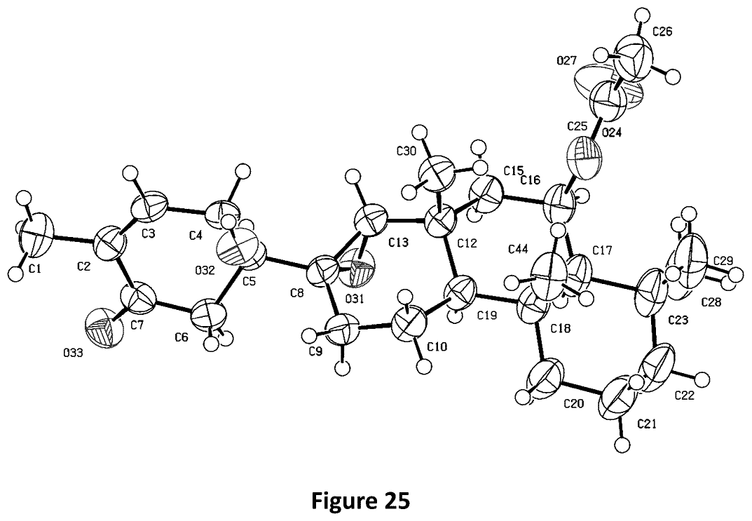

A summary of NMR data for suberitenone E (2) is shown in the table of FIG. 16 with NMT spectra and MS data shown in FIGS. 17-23. The crystal data for suberitenone E (2) is shown in the table of FIG. 24. The ellipsoid plot of suberitenone E (2) is shown in FIG. 25.

### Suberitenone F (3)

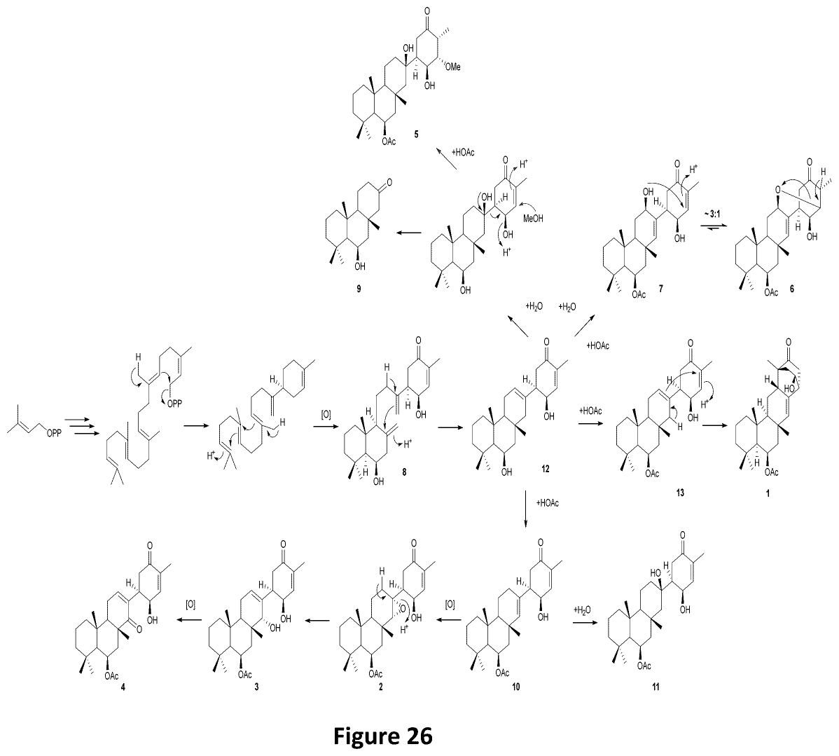

Suberitenone F (3) was isolated as a white film, with a molecular formula C₂₇H₄₀O₅ established by analysis of the formate adduct ion at m/z 489.2841 in the (−)-HRESIMS data and is thus isomeric with suberitenone E (2). The major noticeable differences in the NMR data are related to the resonances of ring C, markedly the presence of an olefinic ¹H resonance (δ_{H} 5.67) that showed COSY correlations to H₂-9 (δ_{H} 2.12). The 2D NMR correlations inferred this signal to be from H-8 and thus suggest a C-7/C-8 double bond as is that of suberitenone C (12) (FIG. 26). Similar to 2, the NMR data gave evidence for an oxymethine at C-22 (δ_{H} 3.15; δ_{C} 75.3) and therefore placed a hydroxyl group at this center, completing the planar structure of 3. Correlations in the NOESY spectrum were used to deduce the chiral centers, where the key correlation between H-22 and H₃-23 (δ_{H} 0.99) showed these two to be related syn to each other, leading to the 22R assignment. All other observed correlations confirmed the remaining configurations to be the same as those deduced by XRD on 2.

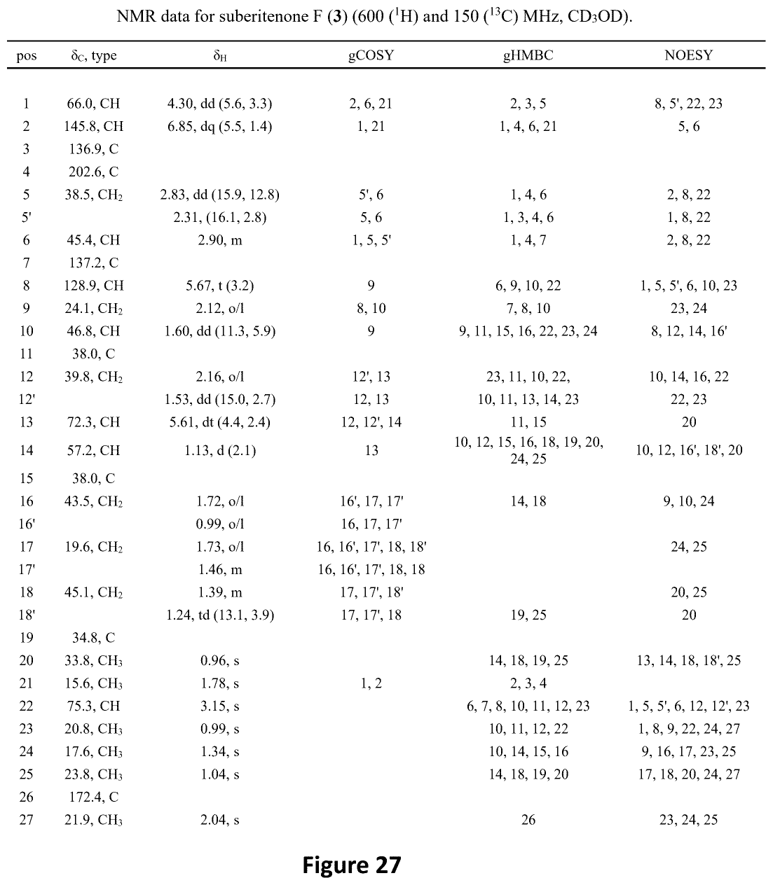

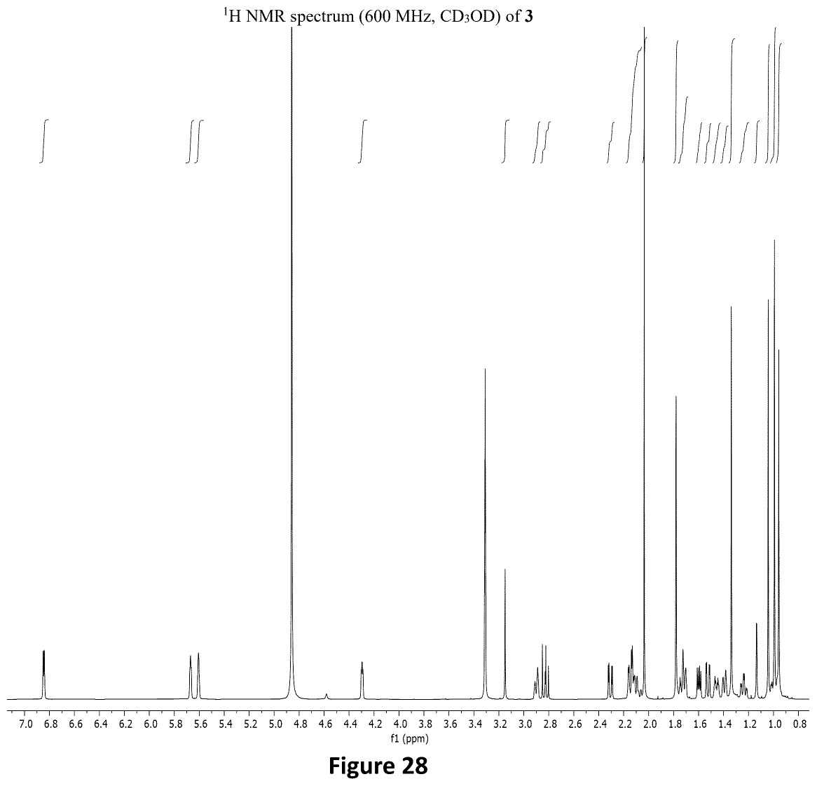

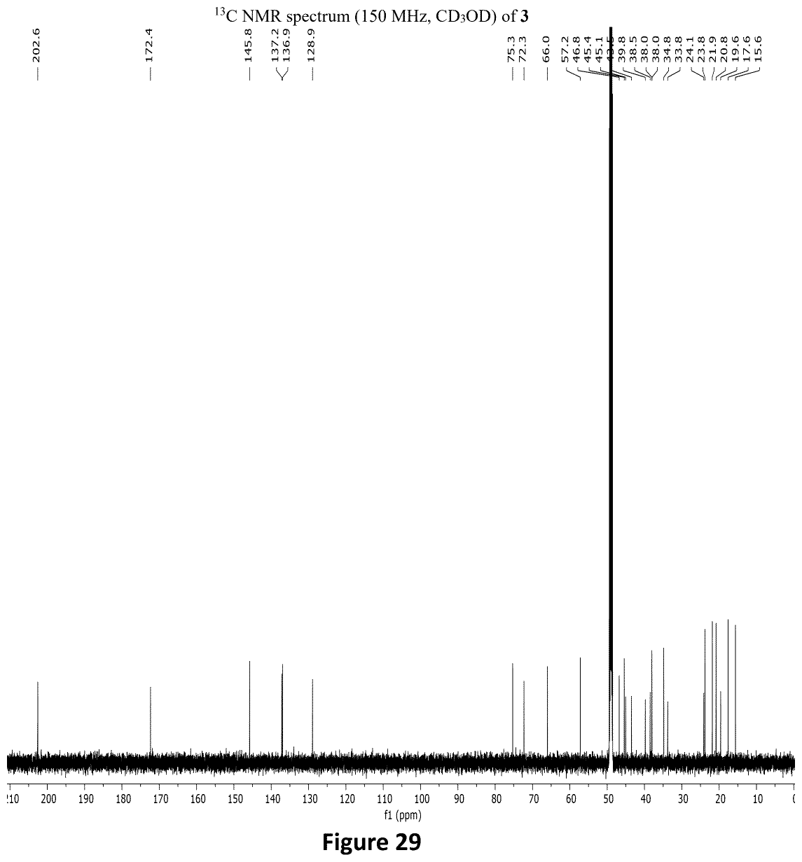

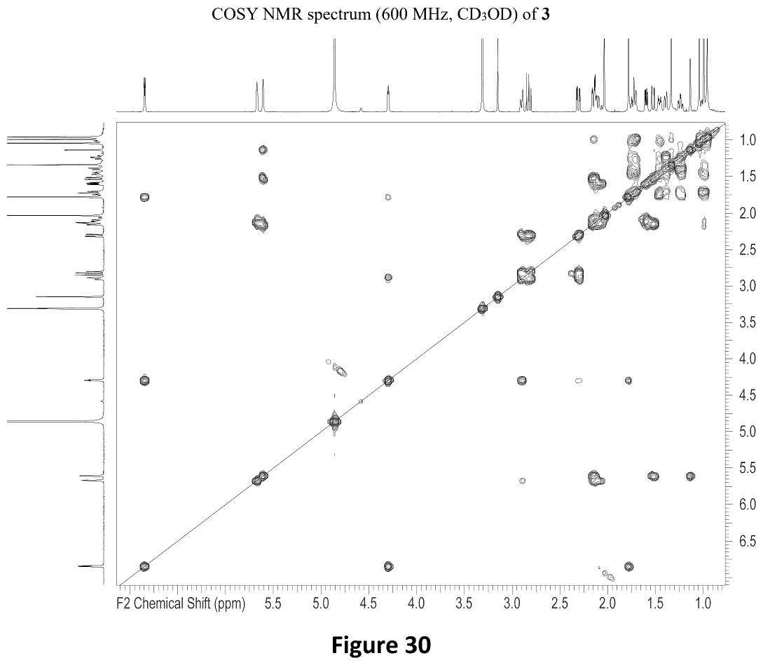

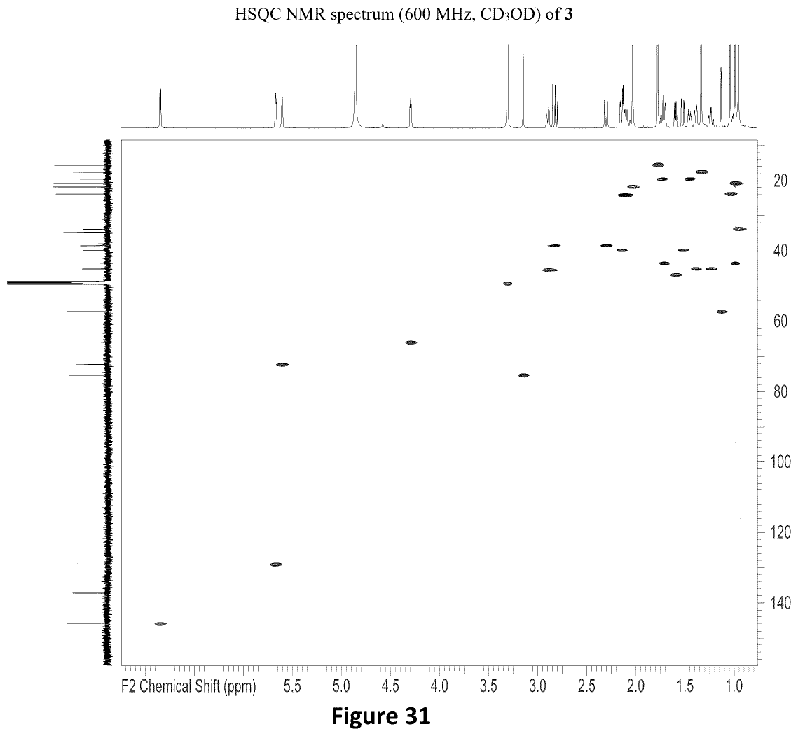

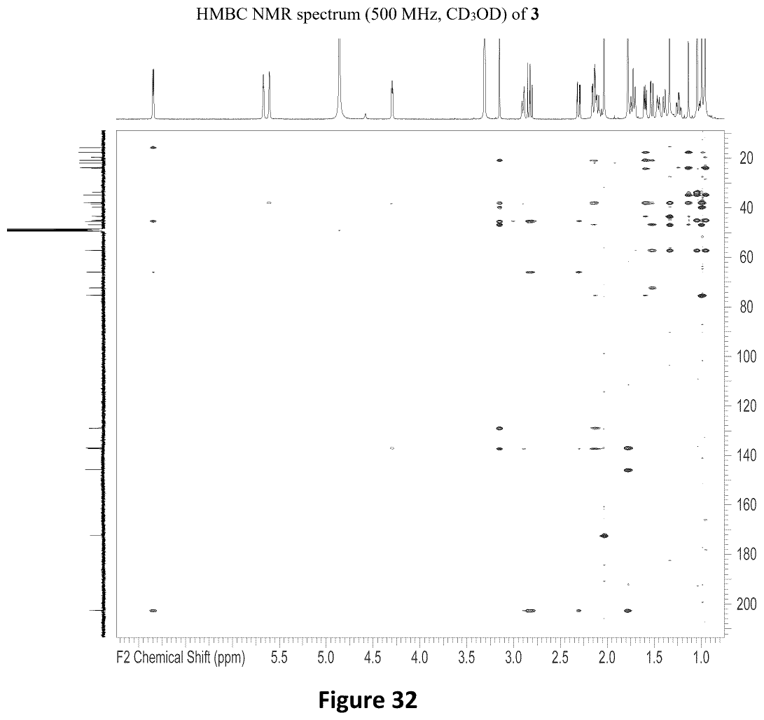

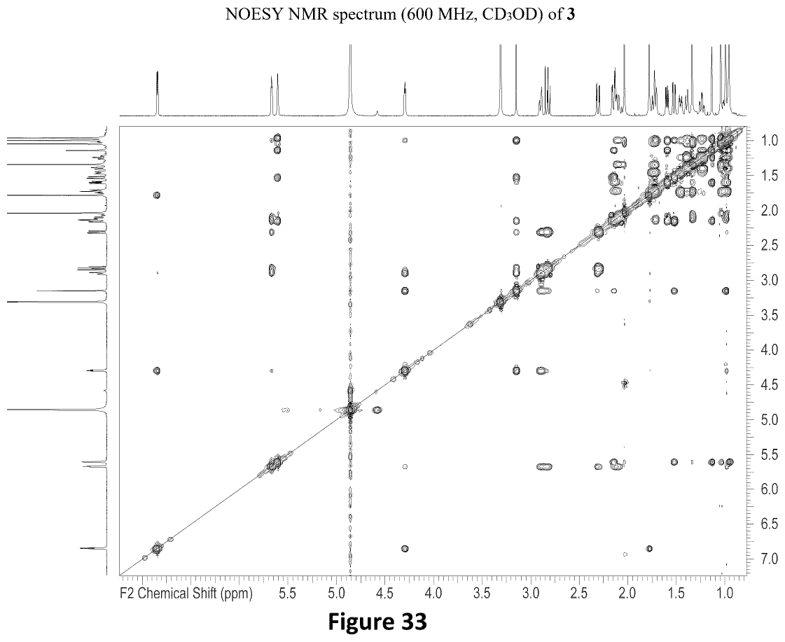

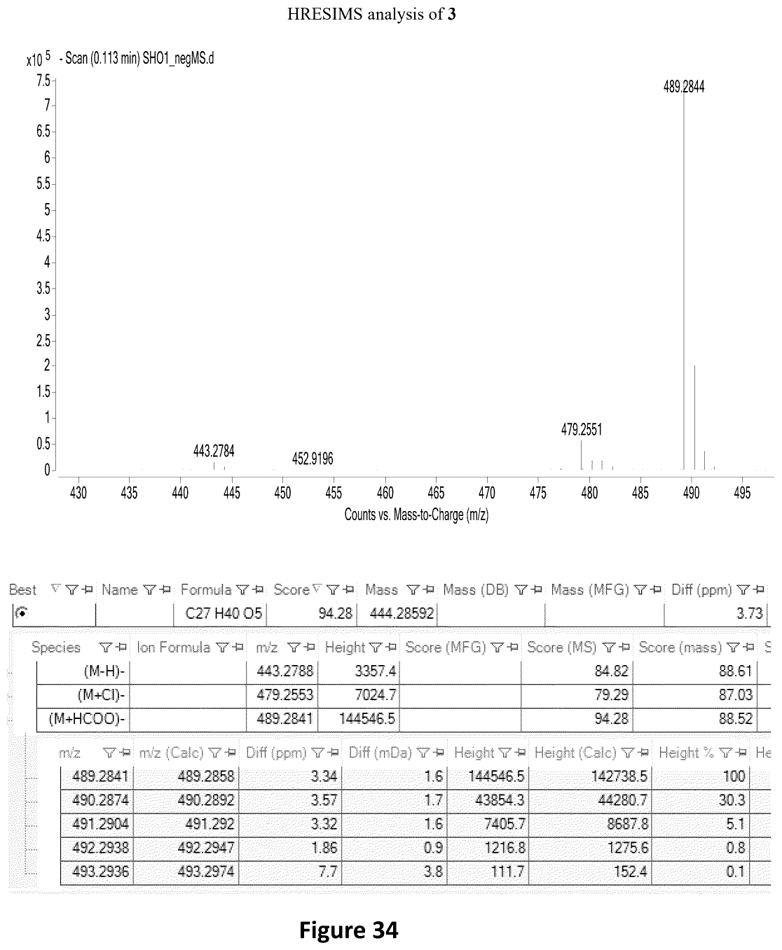

A summary of NMR data for suberitenone F (3) is shown in the table of FIG. 27 with NMT spectra and MS data shown in FIGS. 28-34.

### Suberitenone G (4)

Suberitenone G (4) was isolated as a white film, with a molecular formula C₂₇H₃₈O₅ established by analysis of the deprotonated molecule at m/z 441.2645 in the (−)-HRESIMS data. The NMR data (in MeOD as decomposition was observed in CDCl₃) for 4 suggested the molecule to be related to suberitenone A (9), however the C ring olefin resonances were further deshielded moving from δ_{H} 5.18; δ_{C} 138.9 to δ_{H} 6.78; δ_{C} 147.2. Combined with the observation of a second α,β-unsaturated ketone (δ_{C} 205.5) that showed HMBC correlations from this new olefin methine, suggested 4 contained an enone in ring C as well as ring D. A HMBC correlation from H₃-23 (δ_{H} 1.27) to the new ketone placed it as C-22, while COSY correlations between H-10 (δ_{H} 1.71) and H₂-9 (δ_{H} 2.52) to olefin H-8 (δ_{H} 6.78) confirmed the ring C enone orientation. The configuration of the stereocenters of 4 were deduced from NOESY correlations to be same as that of 2 as was previously deduced by XRD.

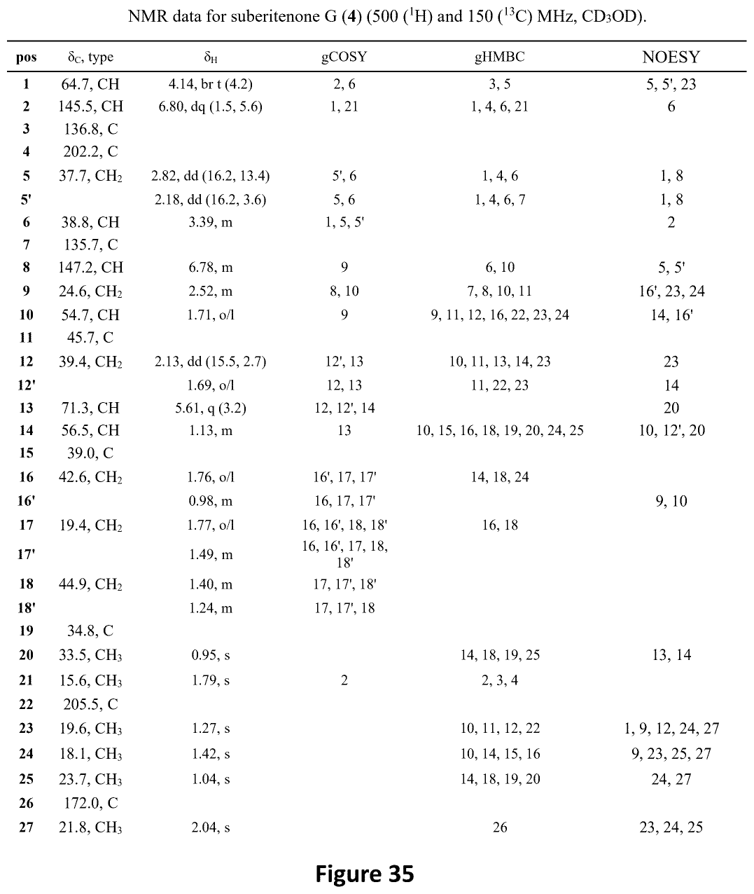

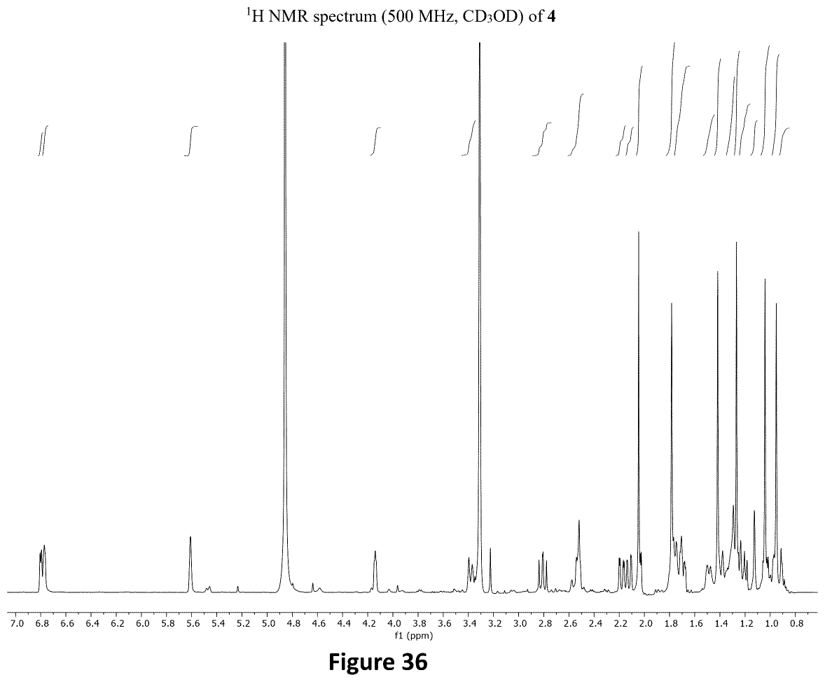

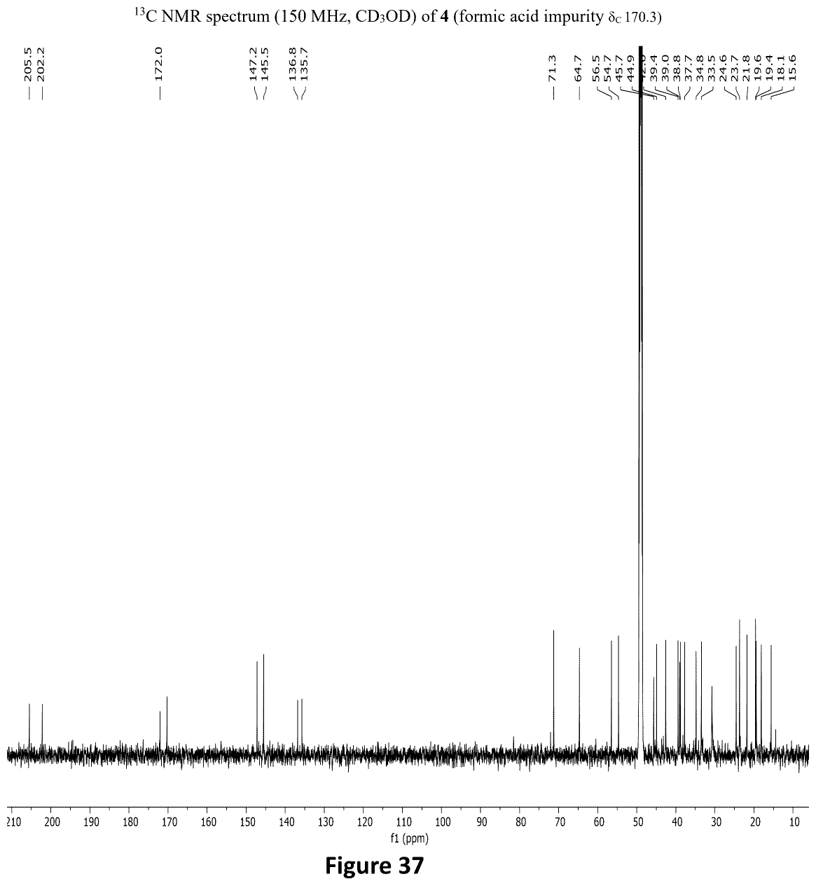

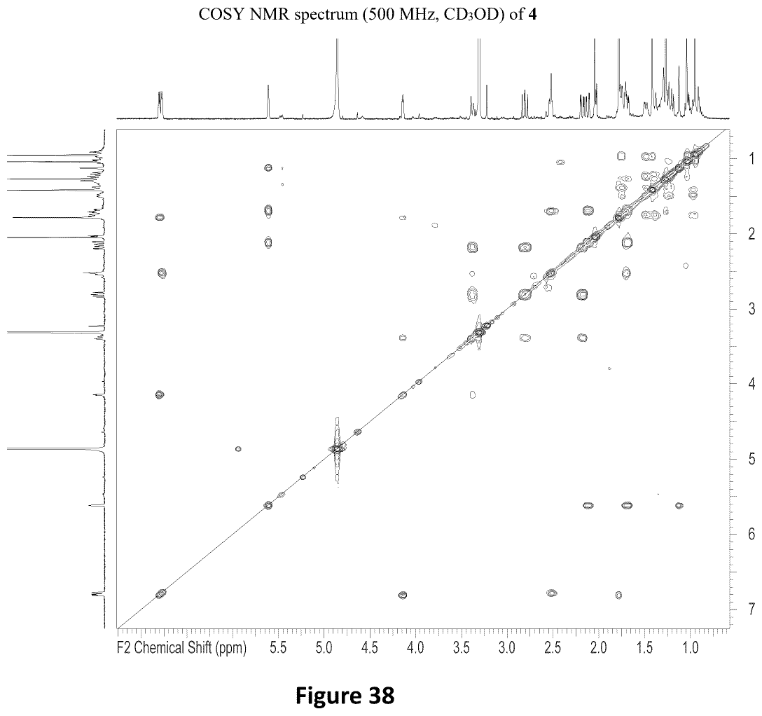

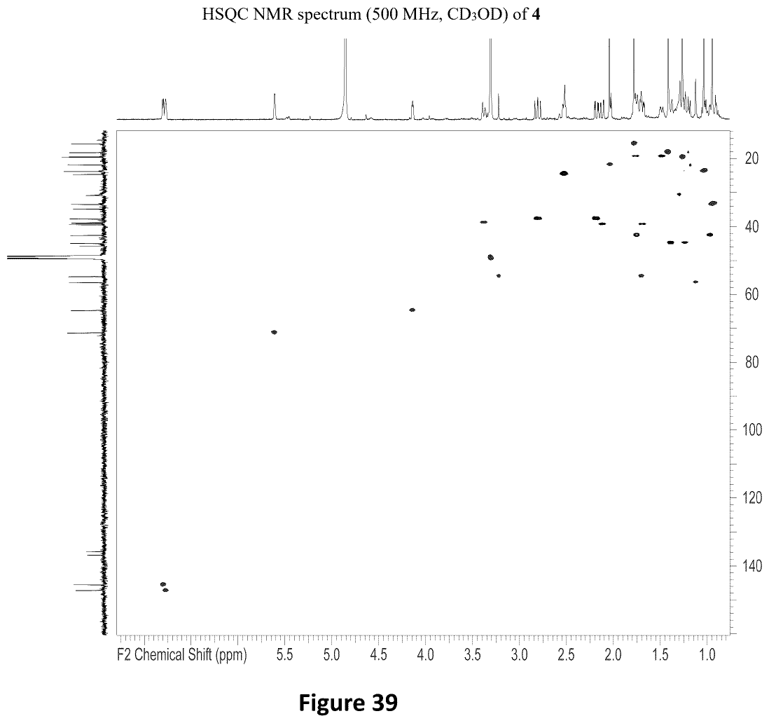

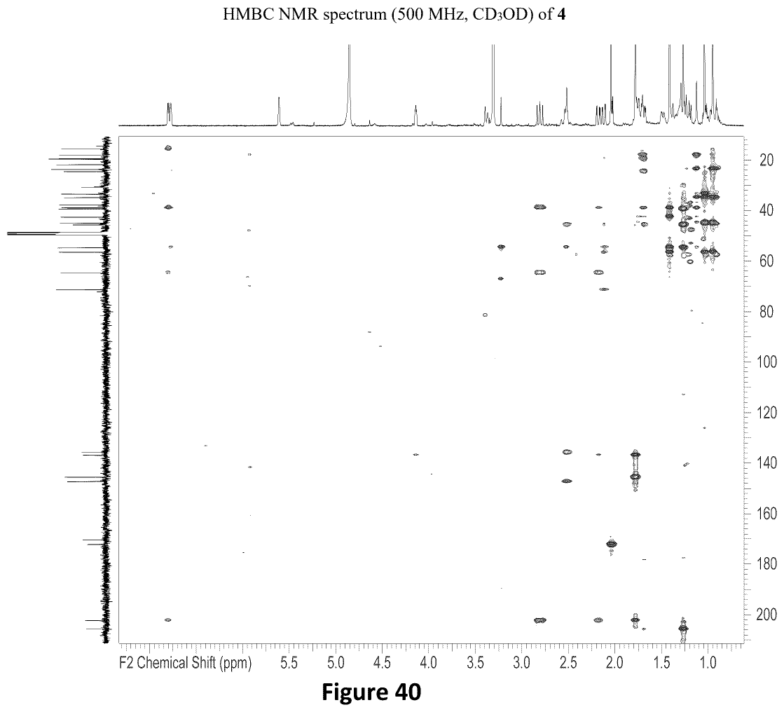

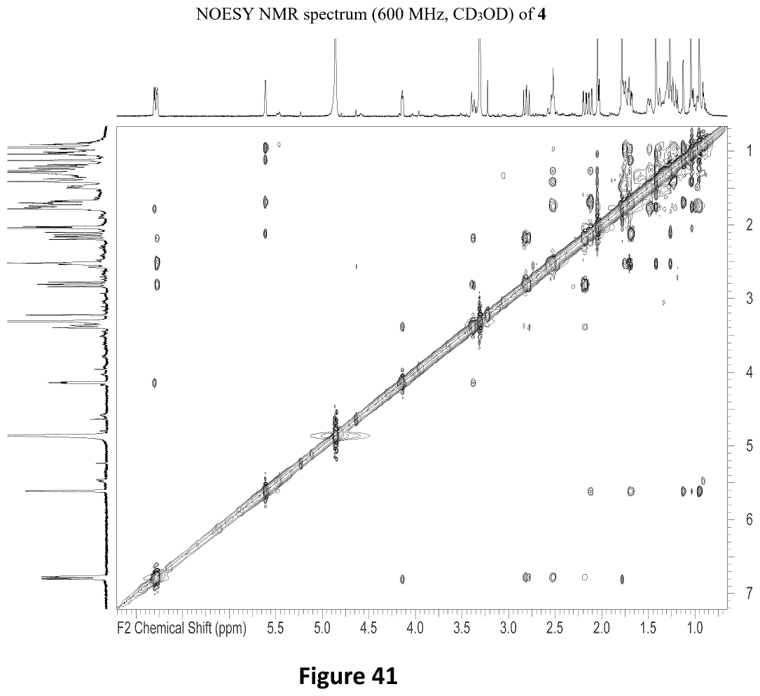

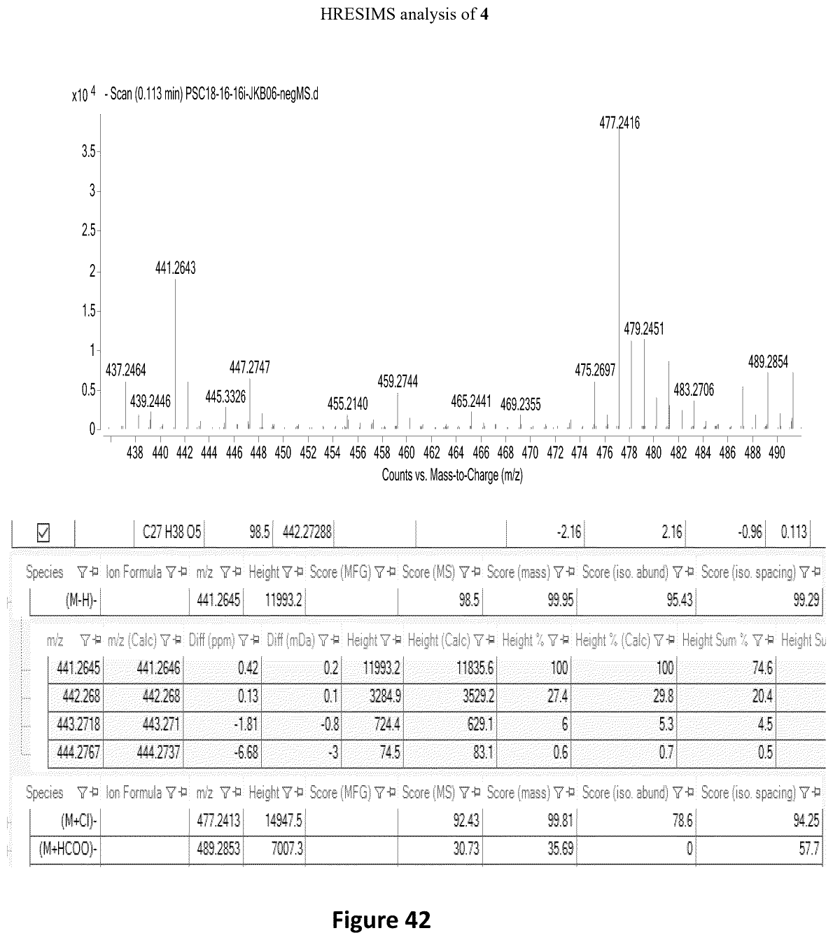

A summary of NMR data for Suberitenone G (4) is shown in the table of FIG. 35 with NMT spectra and MS data shown in FIGS. 36-42.

### Suberitenone H (5)

Suberitenone H (5) was isolated as a white film, with a molecular formula C₂₈H₄₆O₆ established by analysis of the formate adduct ion at m/z 523.3284 in the (−)-HRESIMS data. Analysis of the ¹H and ¹³C NMR data suggested 5 to be similar to suberitenone B (10), hydrated across the C-7/C-22 bond with a tertiary alcohol at C-7. However, 5 exhibited several differences in the resonances associated with ring D including loss of the olefinic ¹³C/¹H signals, splitting of the methyl signal into a doublet and the gain of an aliphatic methine, an oxymethine and a methoxy signal. The doublet methyl H₃-21 (δ_{H} 1.02) correlated in the COSY spectrum to H-3 (δ_{H} 3.03) which correlated with H-2 (δ_{H} 3.52). The signal of H-2 and methoxy H₃-28 showed mutual HMBC to the other carbons for C-28 and C-2 (δ_{C} 59.1 and 88.6 respectively) and this accounts for the loss of the C-2/C-3 double bond. The remainder of ring D remains the same as suberitenone B, assigned on the basis of COSY and HMBC correlations.

The stereochemistry of suberitenone H (5) was deduced by a combination of J coupling constant analysis and correlations in the NOESY spectrum. The similar elements of 5 and suberitenone B (10) were determined to have the same configurations as above, with methyl groups H₃-23 (δ_{H} 1.37), H₃-24 (δ_{H} 1.23) and H₃-25 (δ_{H} 1.04) all deduced to be cofacial with acetyl group H₃-27 (δ_{H} 2.03) by NOESY correlations, while a correlation between H-22 (δ_{H} 1.81) and H₃-23 also placed this proton syn. In contrast, H-22′ (δ_{H} 1.15) showed a NOESY correlation to H-10 (δ_{H} 0.99) which shared a correlation with H-14 (δ_{H} 1.14), placing these two on the opposite side of the molecule. With the large coupling constant (J=13.7) between H-5 (δ_{H} 2.65) and H-6 (δ_{H} 1.81), these protons share a pseudo axial-axial relationship, and as has been seen for previously isolated suberitenones, 12 suggested C-6 (δ_{C} 48.3) to have a S configuration. The relatively smaller coupling constant between H-6 and H-1 (J=3.3) suggested C-1 (δ_{H} 66.6) to also have the S configuration. The cofacial relationship of these two protons is further affirmed by both showing NOESY correlations with methoxy H₃-28, which also correlated to H₃-21 suggesting this group was on the same face. In addition, a NOESY correlation between pseudo-axial H-3 and H-5 provided the final evidence to assign 2S and 3R to the ring. The last chiral center, C-7, is a tertiary alcohol and was assigned R configuration, the same as that for suberitenone B. The chemical shift is consistent with the previously isolated molecules, while NOESY correlations between H-1 and H-22′, and H-8′ with both H-5′ and H-6 mirrors those used diagnostically for isosuberitenone B.¹²

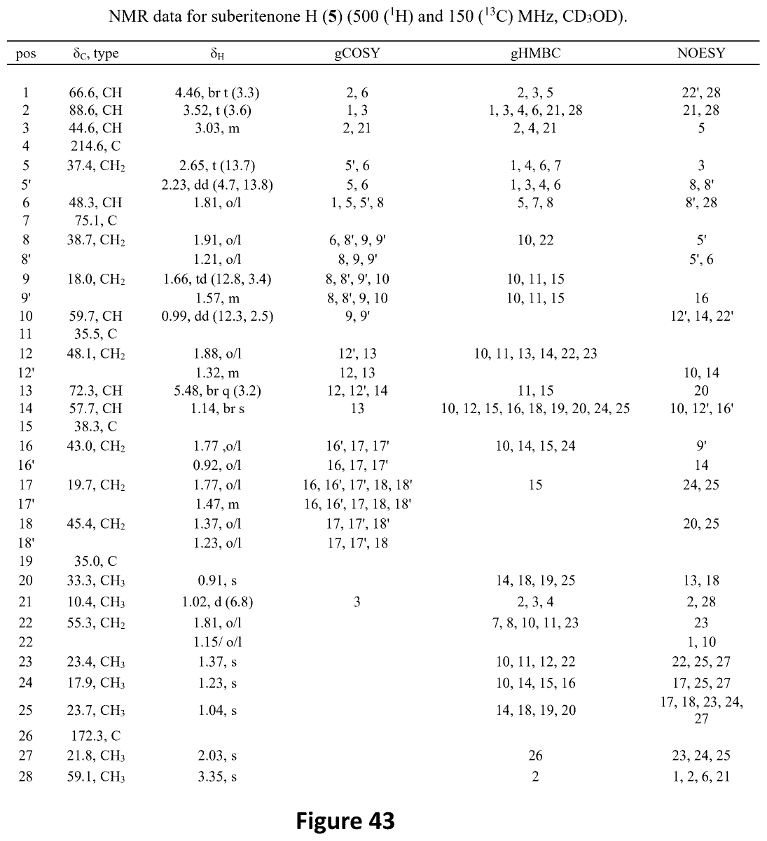

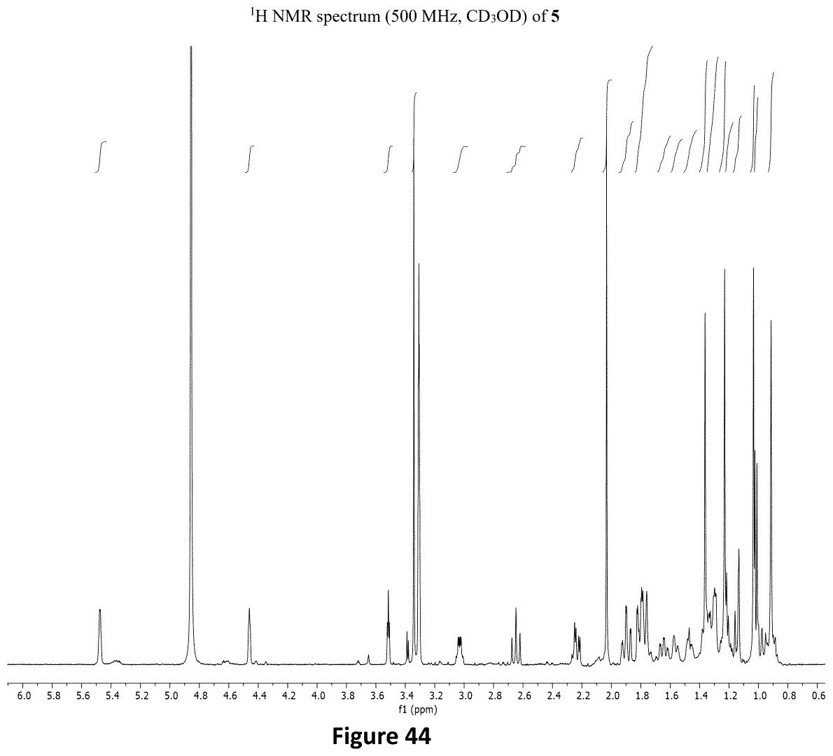

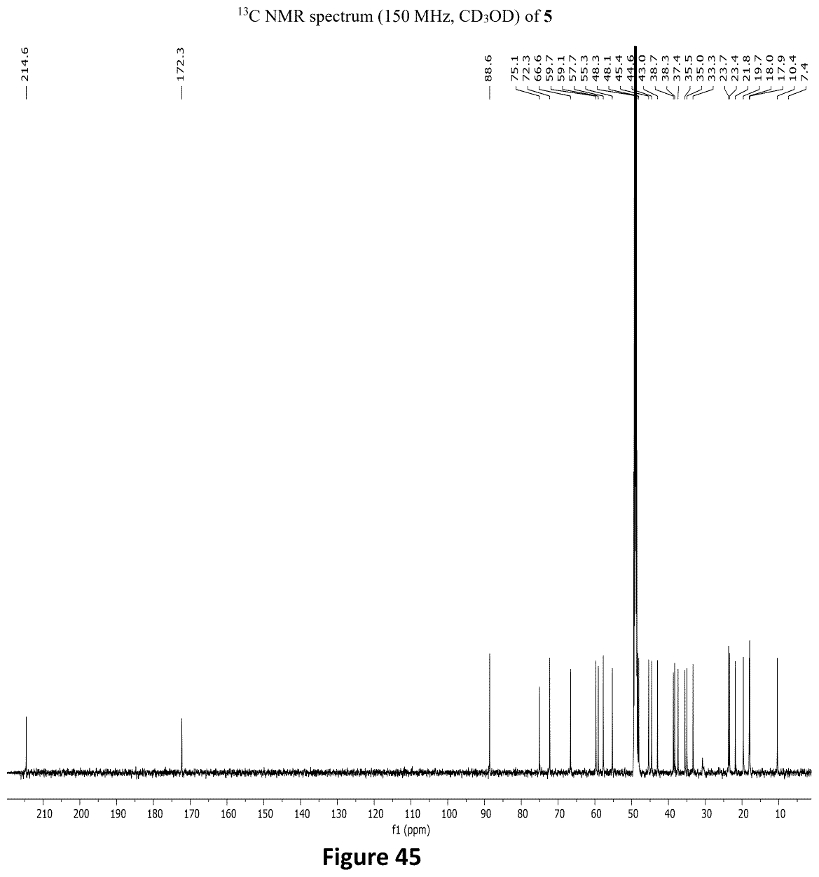

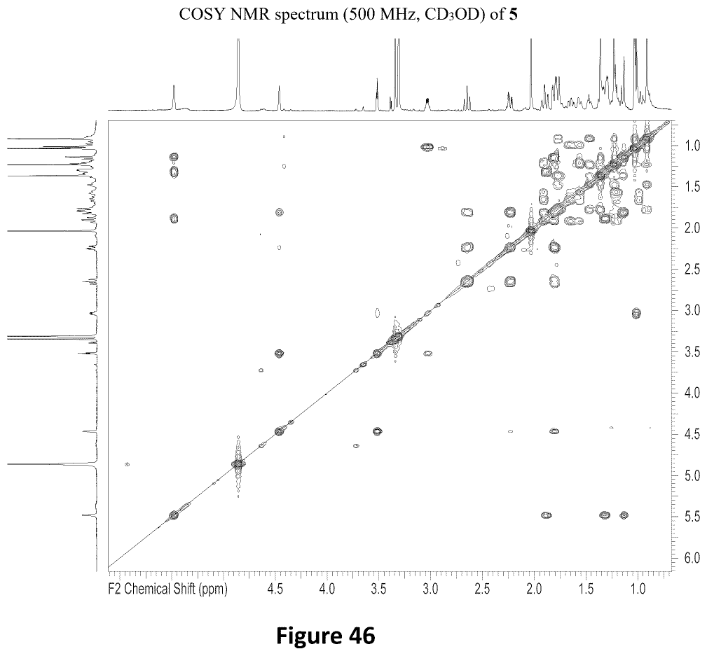

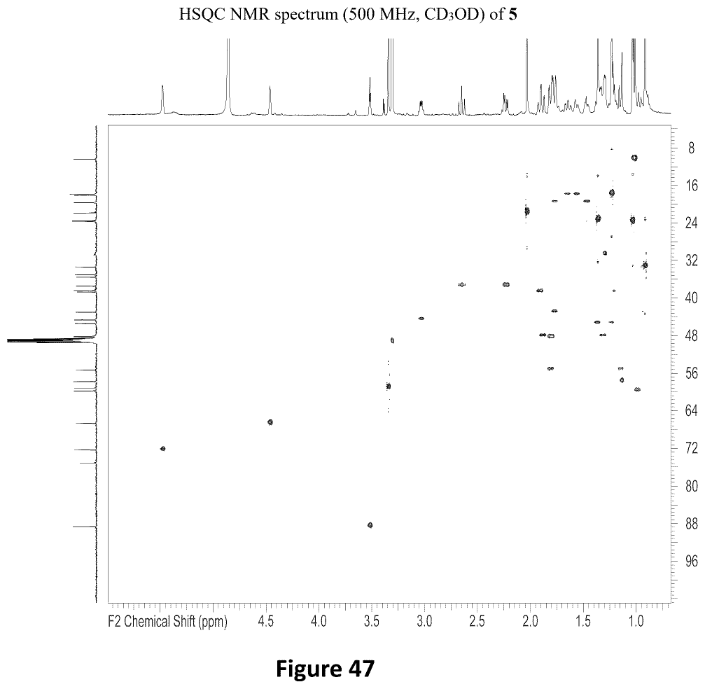

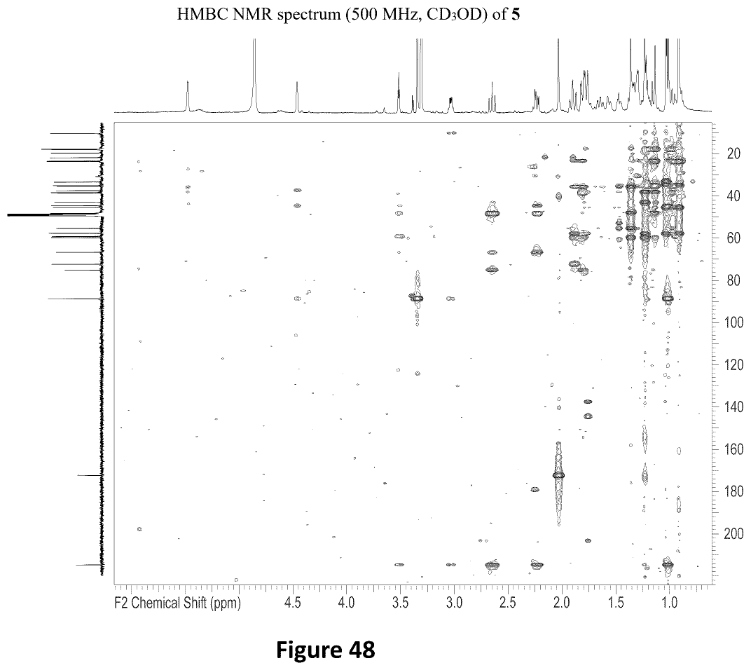

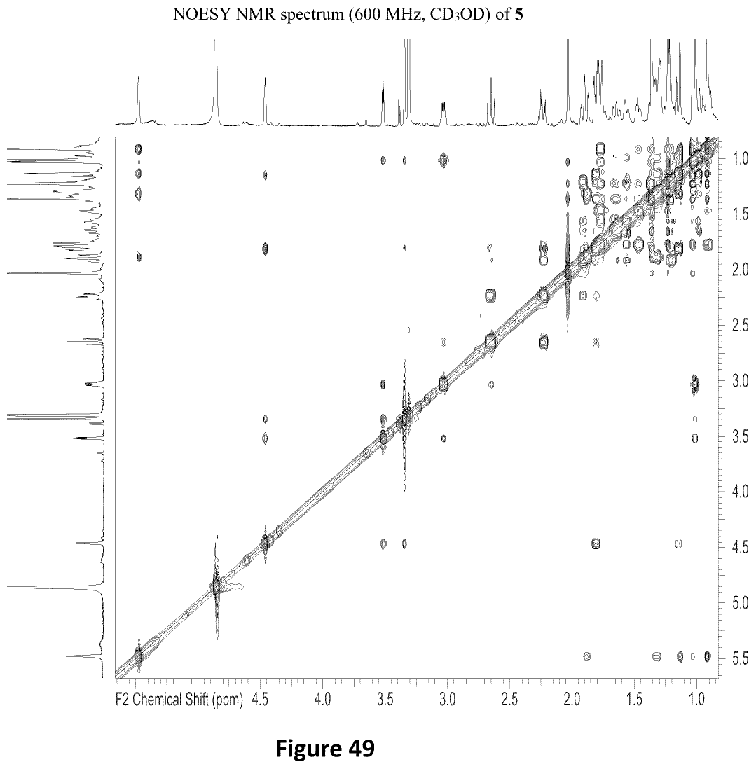

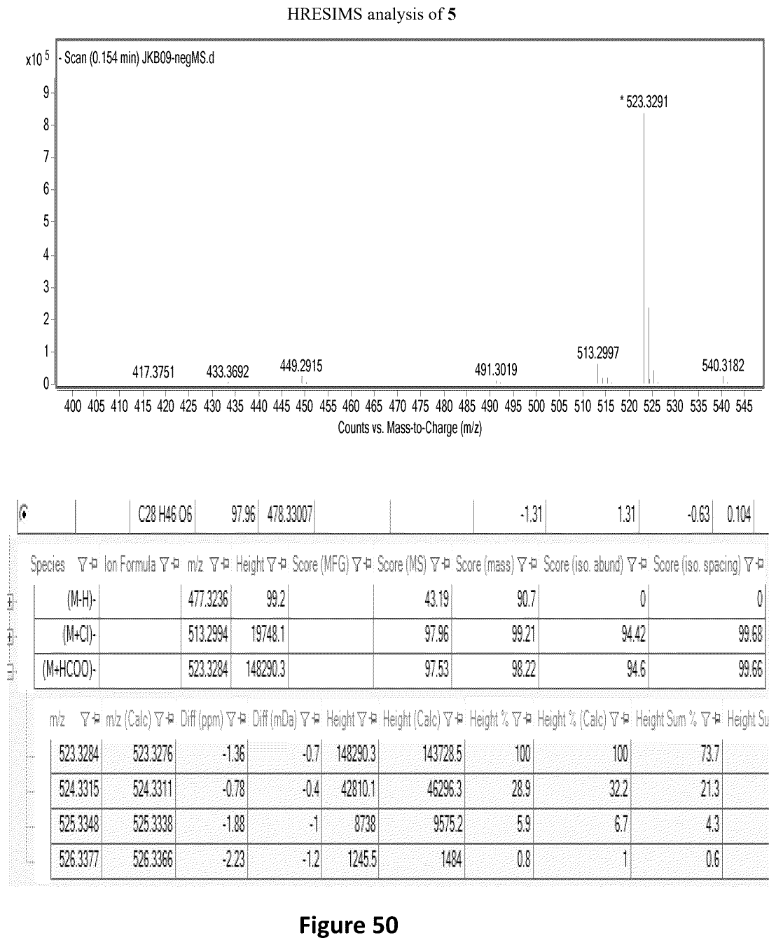

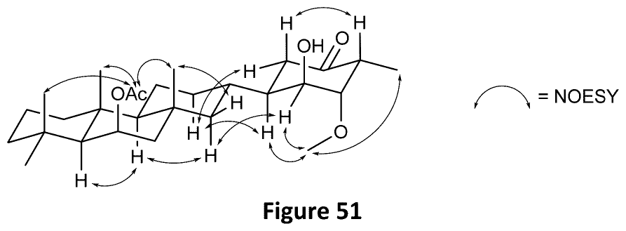

A summary of NMR data for suberitenone H (5) is shown in the table of FIG. 43 with NMT spectra and MS data shown in FIGS. 44-50. NOESY correlations of the relative stereochemistry of suberitenone H (5) are shown in FIG. 51.

### Suberitenone l (6)

Suberitenone I (6) was isolated as a white film with a molecular formula C₂₇H₄₀O₅ established by analysis of the formate adduction at m/z 523.3284 in the (−)-HRESIMS data, therefore isomeric with 2. Like suberitenone H (5), the ¹H and ¹³C NMR data showed no evidence of a ring D double bond, and instead the signal for H₃-21 (δ_{H} 1.19) was a doublet, and the chemical shift of C-2 and H-2 (δ_{C} 79.9, δ_{H} 4.03) suggested the center was oxygenated. Based on COSY correlations throughout the spin system with H-10 (δ_{H} 1.13) and H₂-9 (δ_{H} 1.90 and 1.67), H-8 was assigned (δ_{H} 3.79, δ_{C} 70.8) and both the ¹³C and ¹H chemical shifts suggest the center was also oxygen bearing. The planar structure of 6 was finally deduced by a key HMBC correlation from H-2 to C-8, thus providing evidence for a oxa-bicyclo[3.3.1]nonane moiety and providing the correct number of degrees of unsaturation.

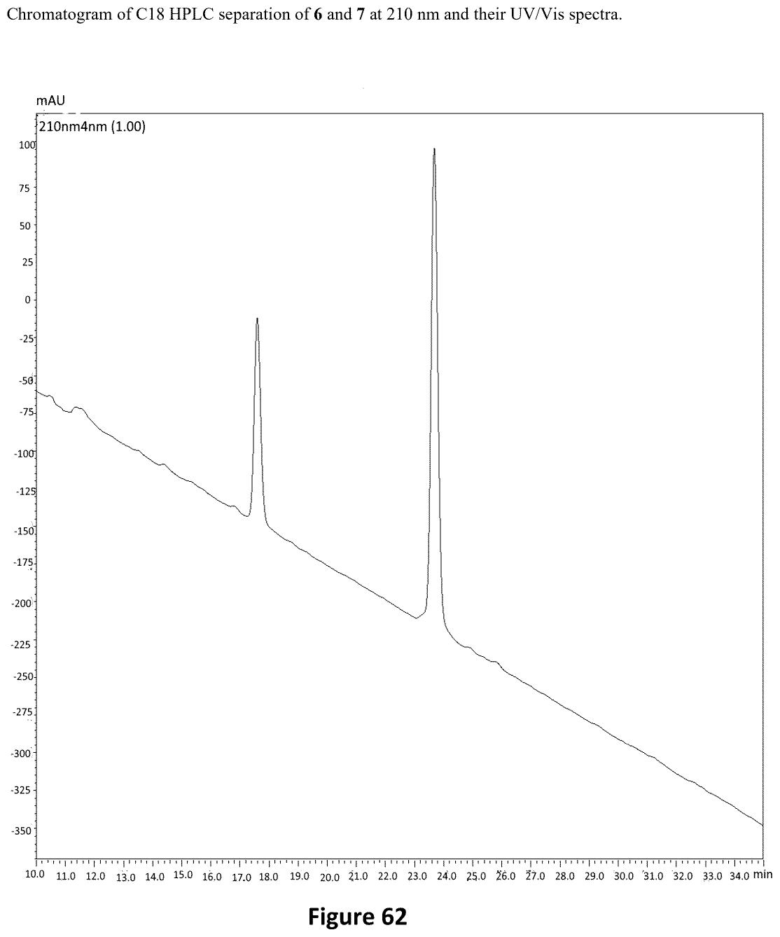

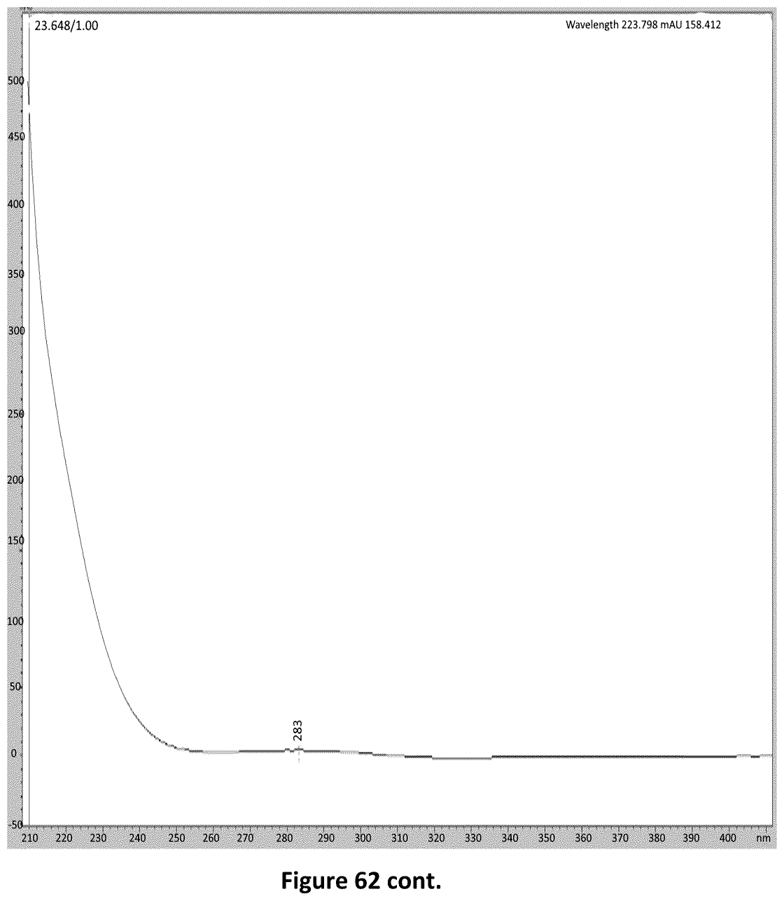

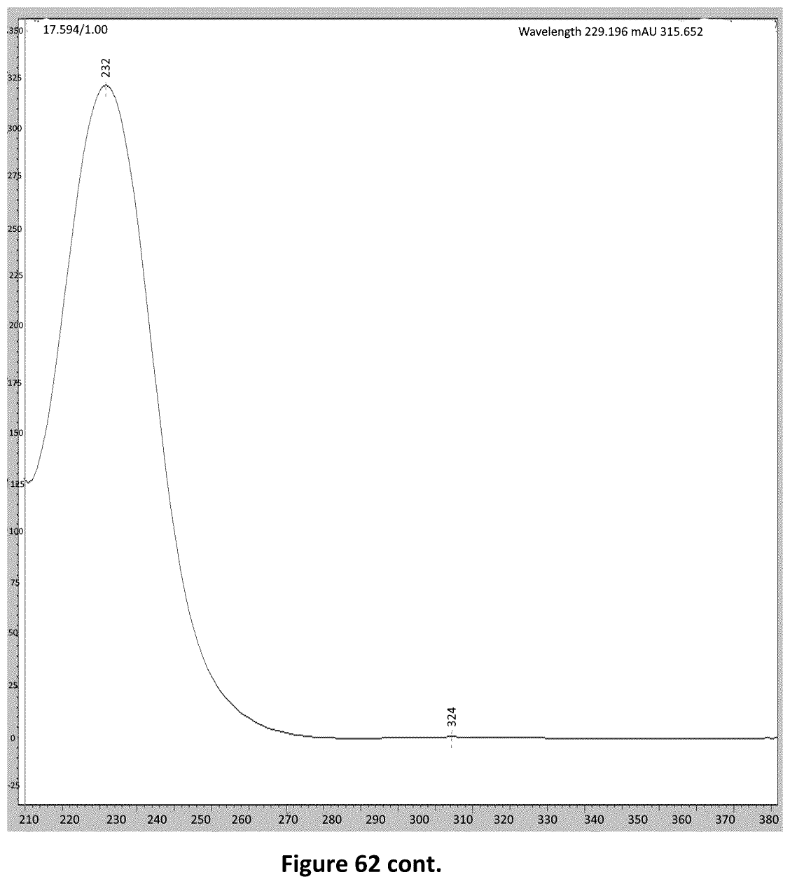

While NMR data was collected on purified suberitenone I (6), the signals decreased in intensity while those of a new species grew in. This more-polar compound was separable by reversed-phase HPLC (FIG. 62), and from analysis of the signals, it was clear the new species was a suberitenone type molecule, with the same enone D ring as suberitenone A (9), and COSY/HSQC correlations revealed C-8 (δ_{H} 4.30) to be hydroxylated. Thus suberitenone J (7) is the oxaspiro-ring opened isomer, existing in equilibrium with 6. When the two were separated by reversed-phase HPLC, both reverted to an equilibrium mixture of ˜3:1 6:7 over 24 h.

To determine the configuration of the chiral centers, key NOESY correlations and the ¹H NMR coupling constants for both compounds were taken into account. In the NOESY spectra of both compounds, a key correlation between H-8 and H-10 placed these two protons syn, establishing the orientation of C-8 relative to the suberitane scaffold. As with oxaspirosuberitenone, the extra ring distorts the D ring away from a pseudo-chair conformation in suberitenone I (6) and thus coupling constants cannot be understood to rapidly assign configurations. This is not the case for suberitenone J (7) where the pseudo axial-axial coupling constant of H-5 and H-6 (J=13.7 Hz) established that C-6 shares the same configuration as the previously isolated congeners, and the same can be applied between H-6 and H-1 (J=4.3 Hz) to establish C-1. Finally, correlations to H₃-21 from H-2 was used to assign the orientation of the methyl group on C-3, which is in accordance with the other molecules isolated from this organism.

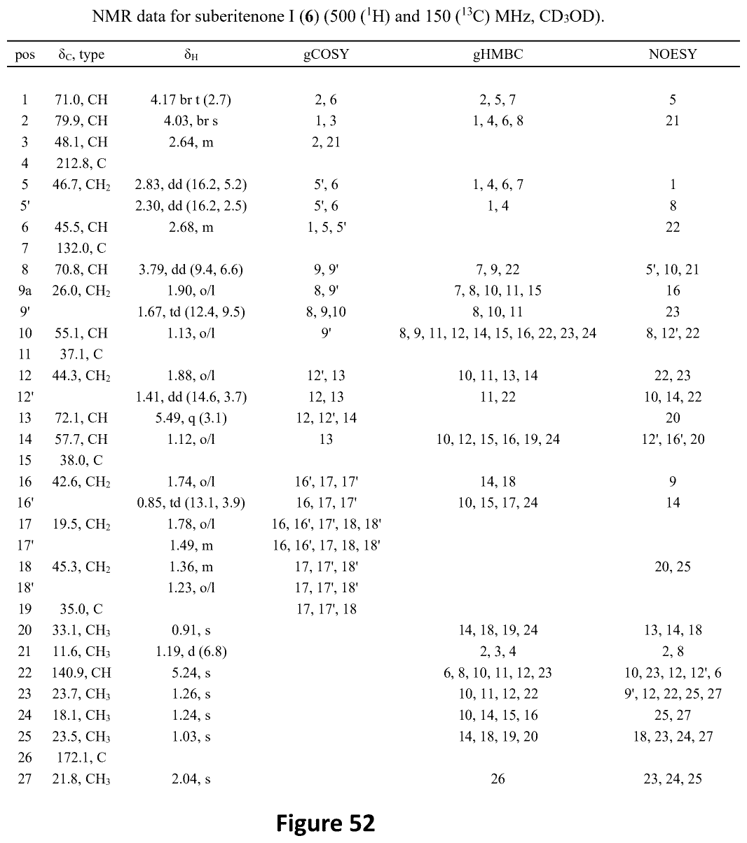

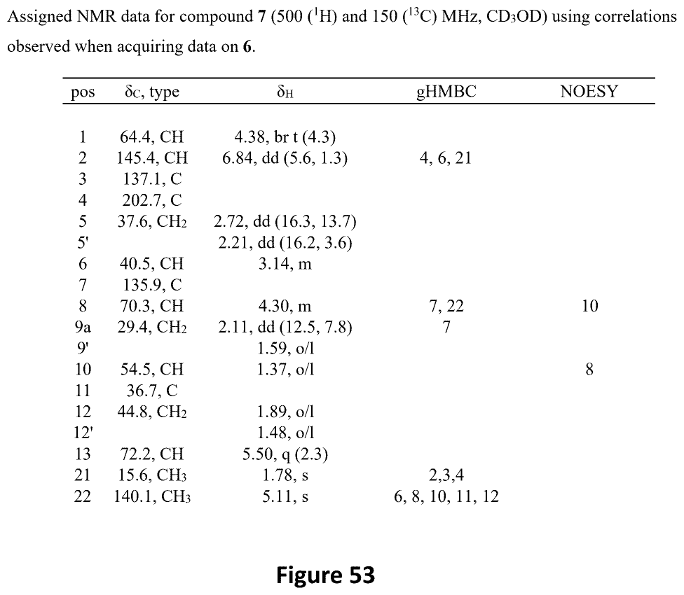

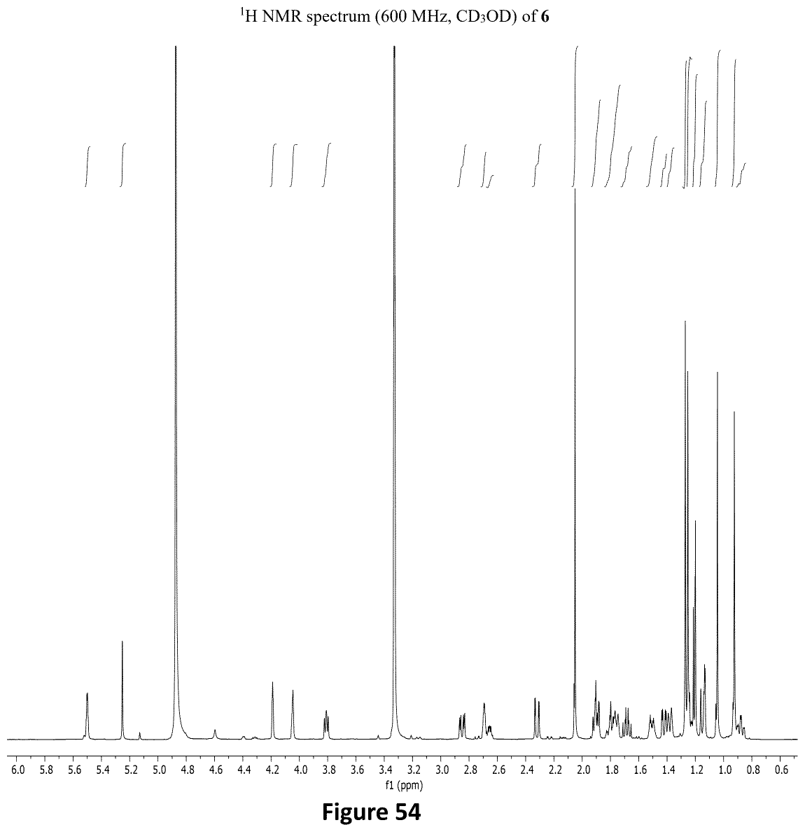

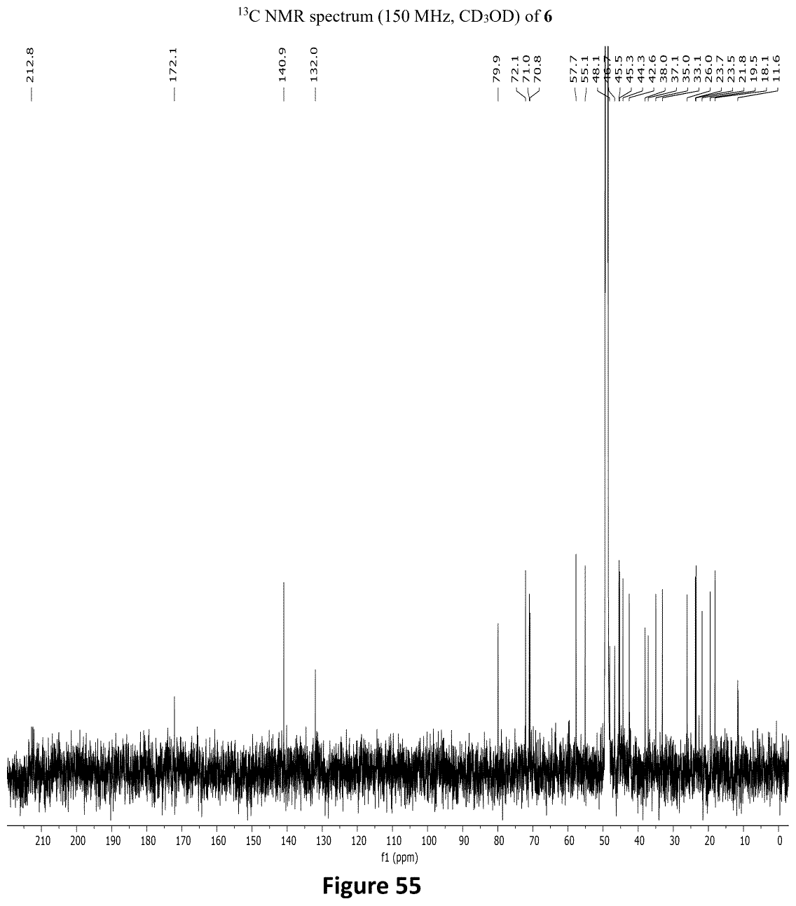

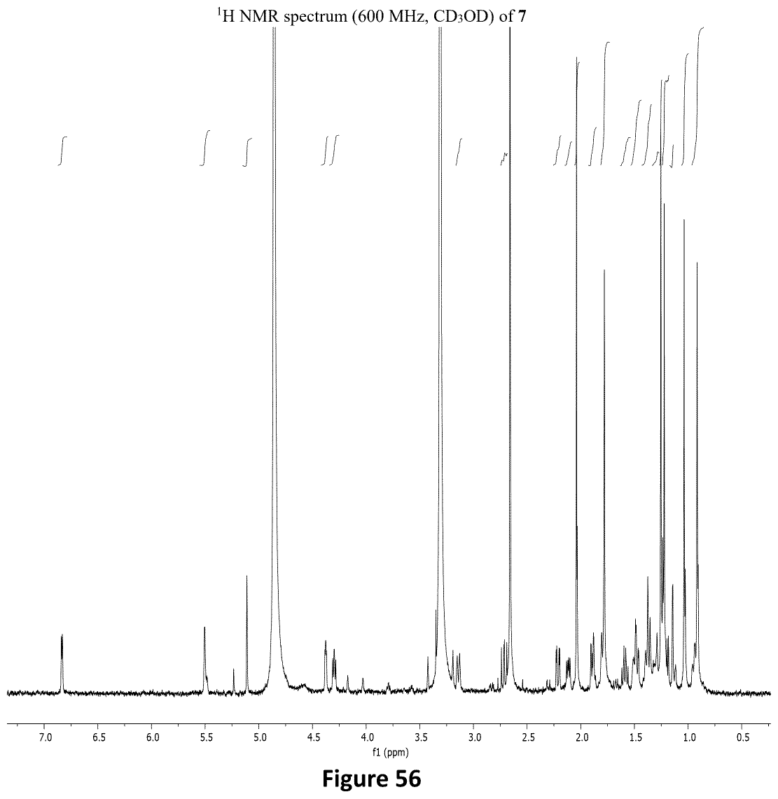

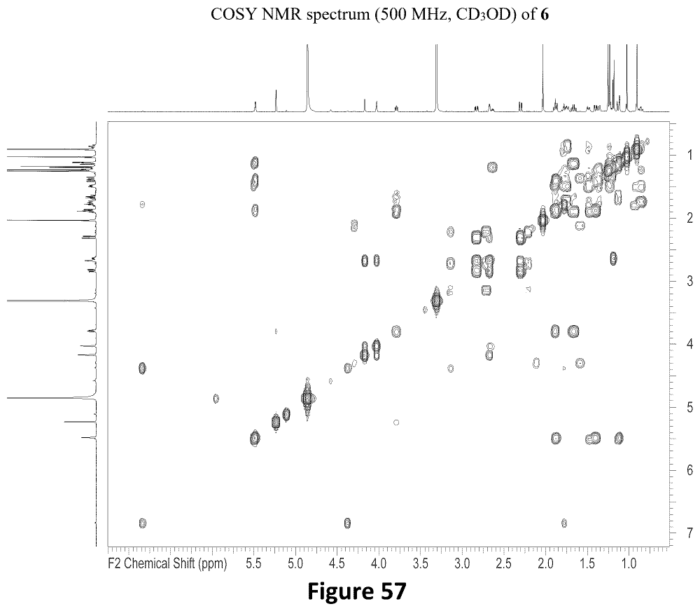

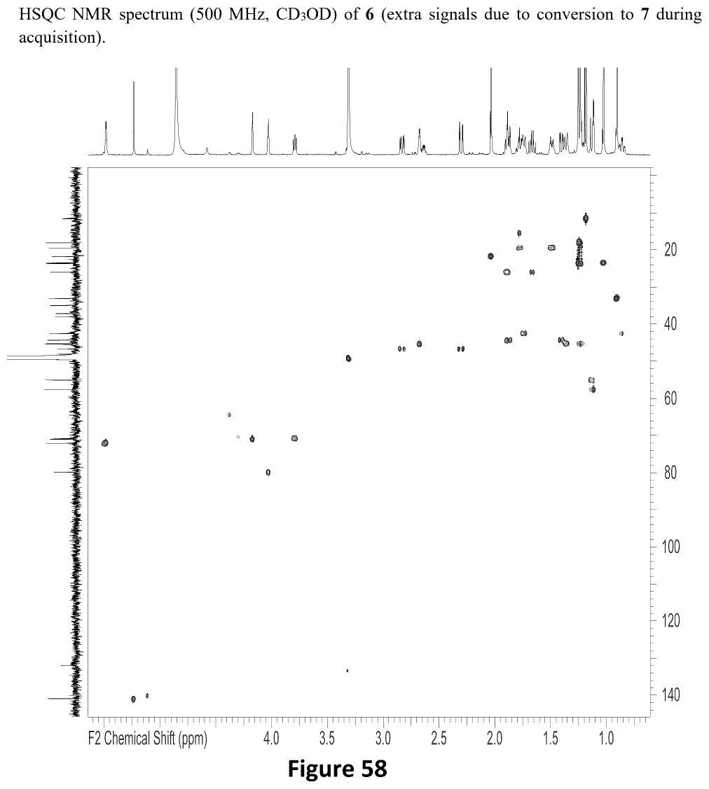

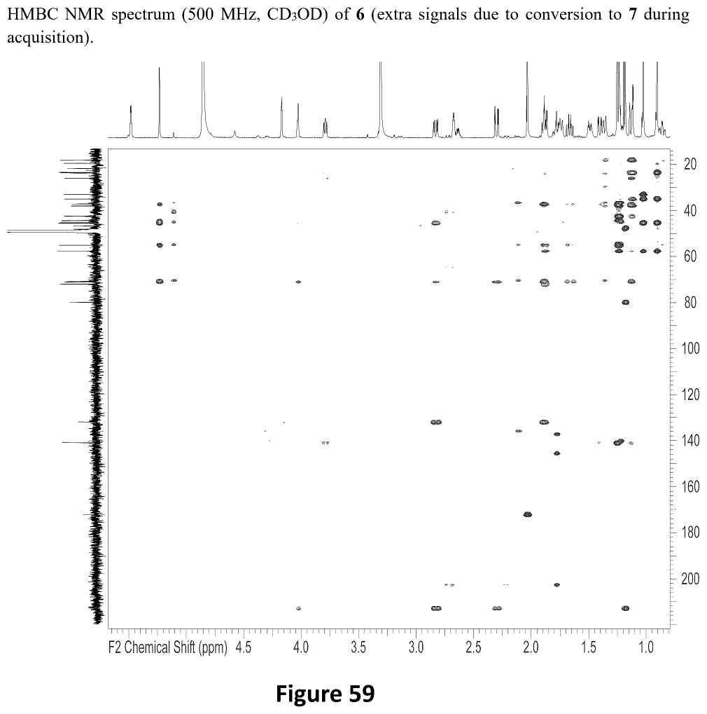

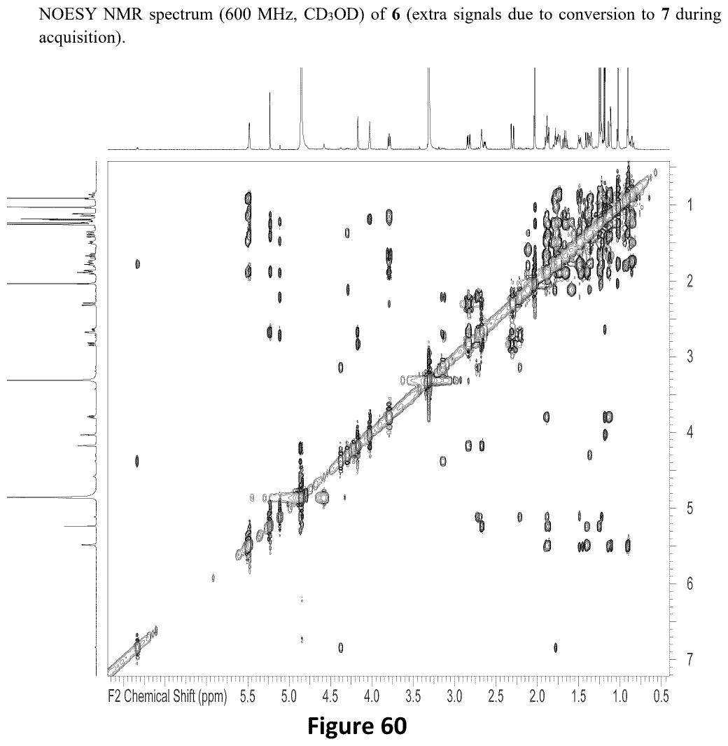

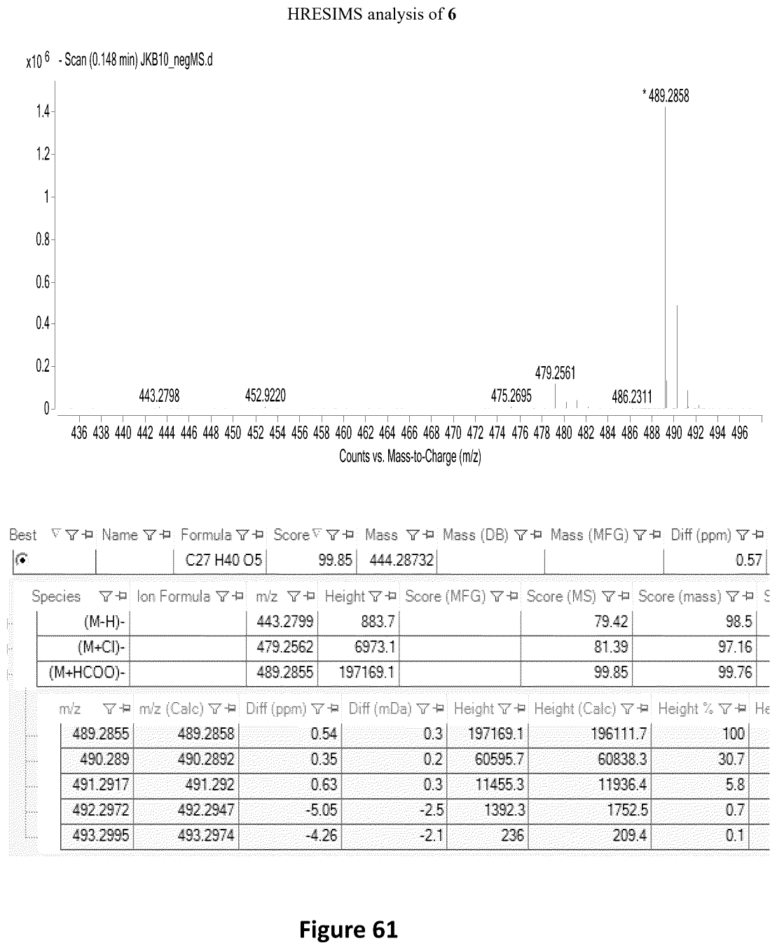

A summary of NMR data for suberitenone I (6) is shown in the table of FIG. 52 and a summary of NMR data for suberitenone J (7) is shown in the table of FIG. 53. NMT spectra and MS data for suberitenone I (6) and suberitenone J (7) are shown in FIGS. 54-62.

### Secosuberitenone A (8)

Secosuberitenone A (8) was isolated as a white film, with a molecular formula of C₂₅H₃₈O₃ established by analysis of the formate adduct ion at m/z 431.2814 in the (−)-HRESIMS data. The 1D and 2D NMR data suggested major differences present in ring C, and also lacked the signals of the acetyl group which suggested ring the ring B hydroxyl did not bear an acetyl group as in the previously isolated metabolites. The most significant difference with other suberitenones was the presence of two terminal vinylidene moieties, thereby accounting for all seven degrees of unsaturation and therefore suggesting that ring C was not cyclized. The ¹H signals for H₂-23 (δ_{H} 5.03 and 4.77) showed HMBC correlations to C-10, C-11 and C-12 (δ_{C} 57.5, 144.4 and 47.8 respectively) therefore this replaced the methyl group normally present there, whereas vinylidene H₂-22 (δ_{H} 5.16 and 4.92) showed correlations to C-1, C-6, C-7 and C-8 (δ_{C} 63.6, 45.5, 148.8 and 34.2 respectively) which revealed it to be adjacent to ring D. Both sets of correlations lead to the conclusion that compound 8 lacks the cyclization bond from C-10 to C-22 typically formed in the terpene backbone cyclization to form the suberitane scaffold (FIG. 26), instead forming the ansellane backbone first observed in ansellone A.¹⁴ The configuration of chiral centers in 8 were deduced from NOESY correlations to be same as that of 2 as was previously deduced by XRD.

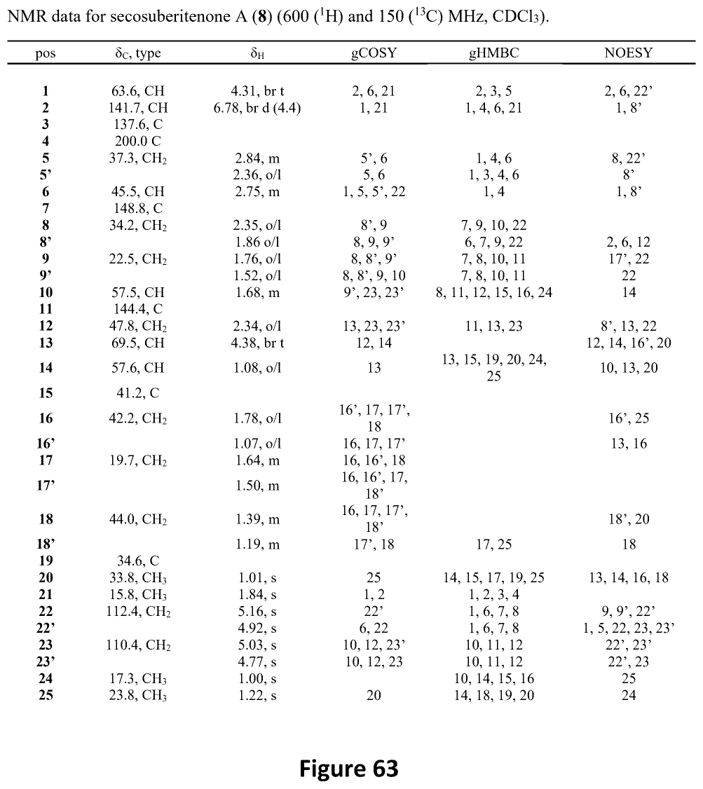

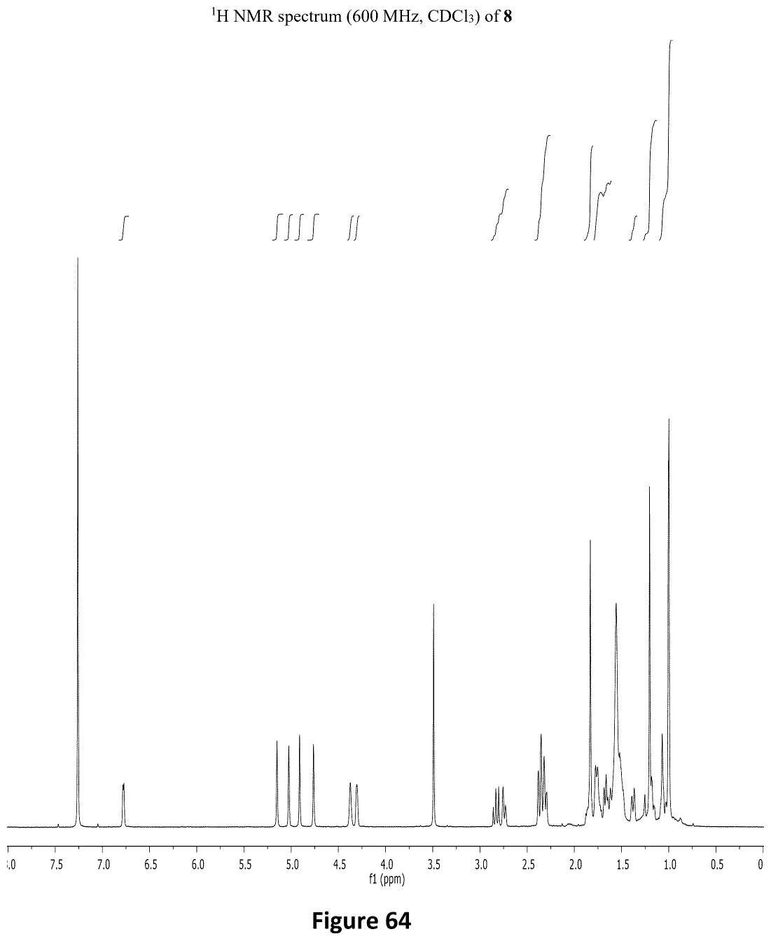

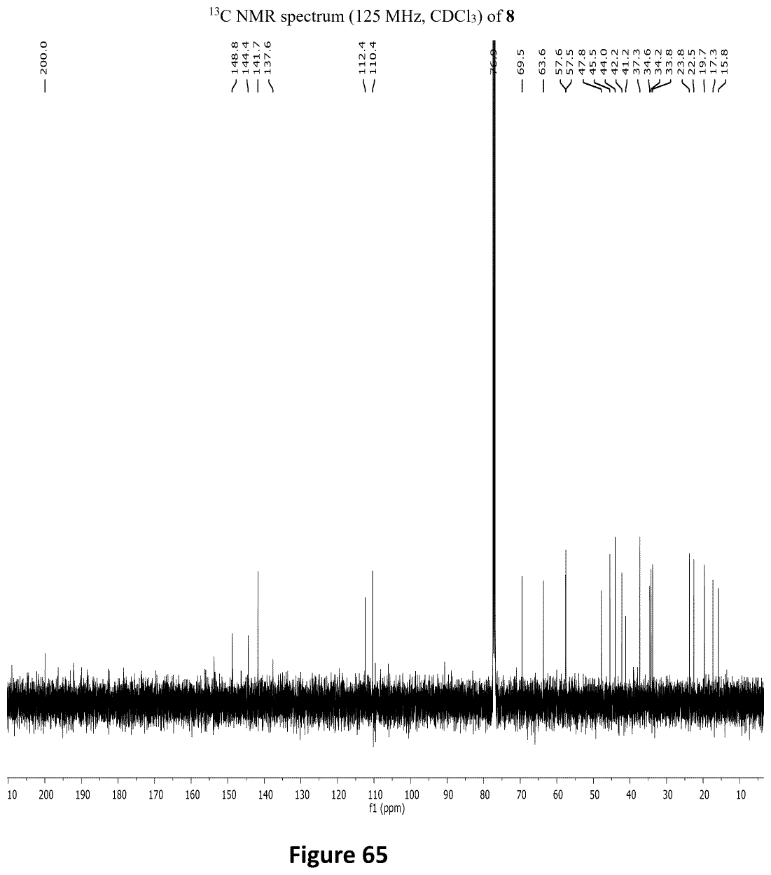

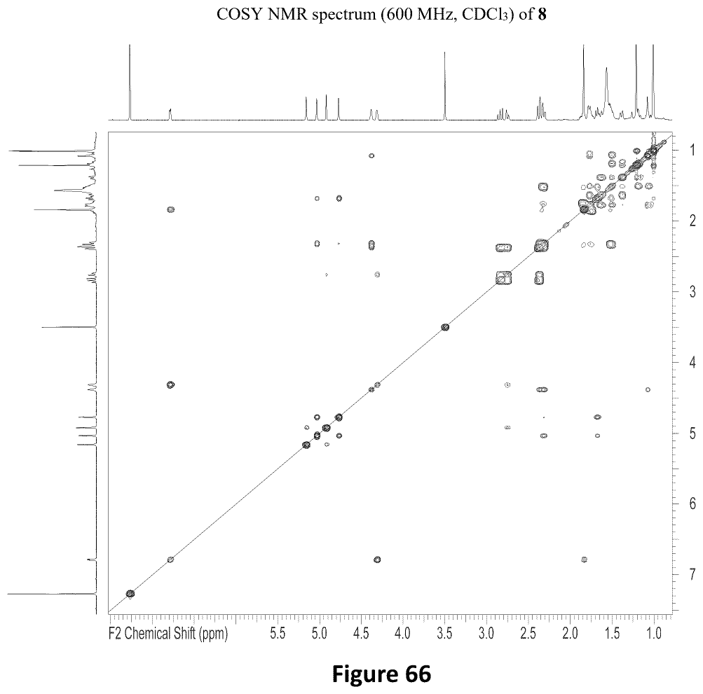

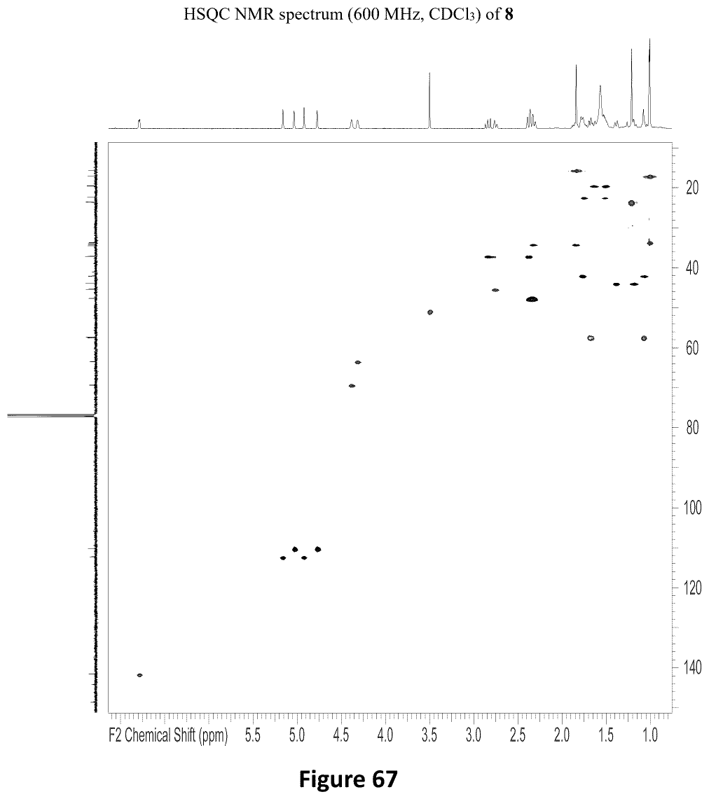

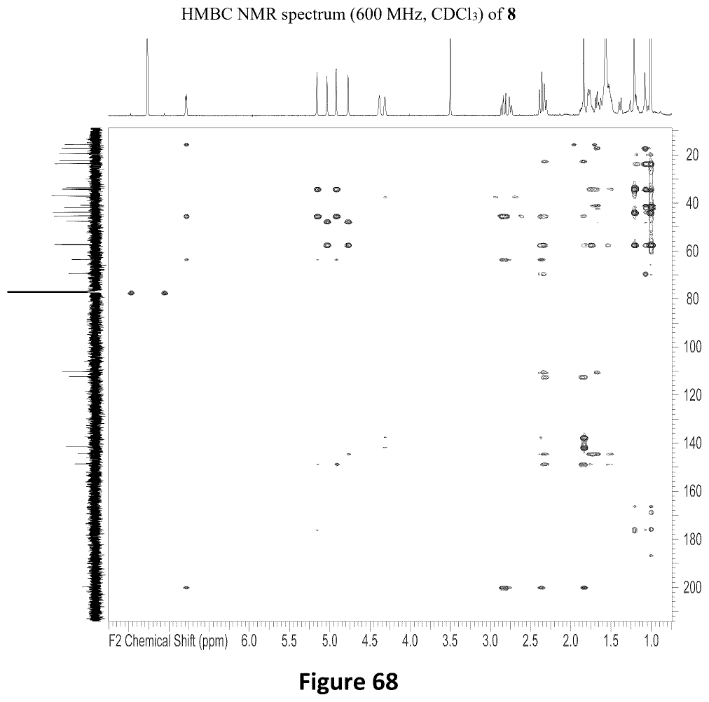

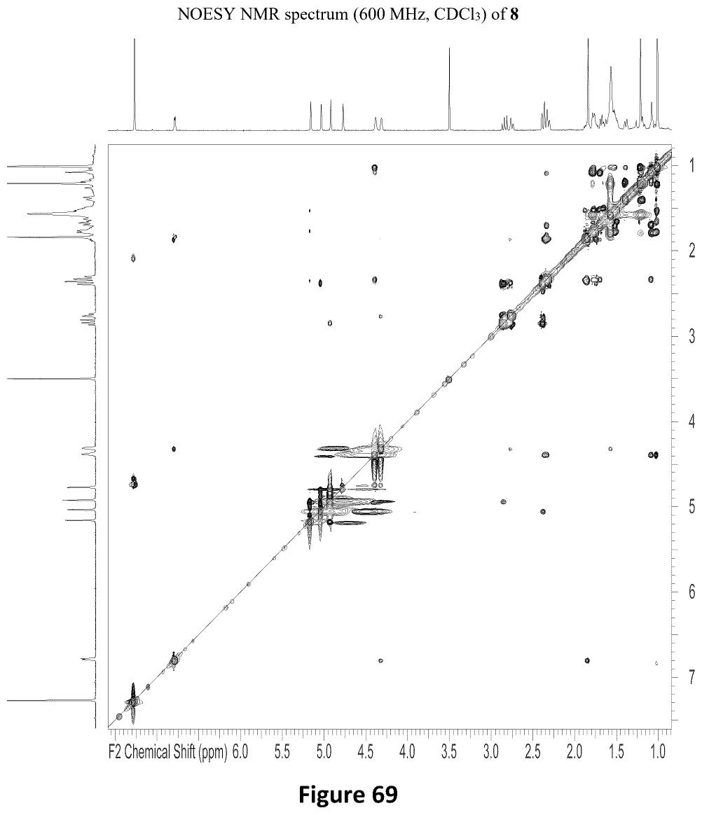

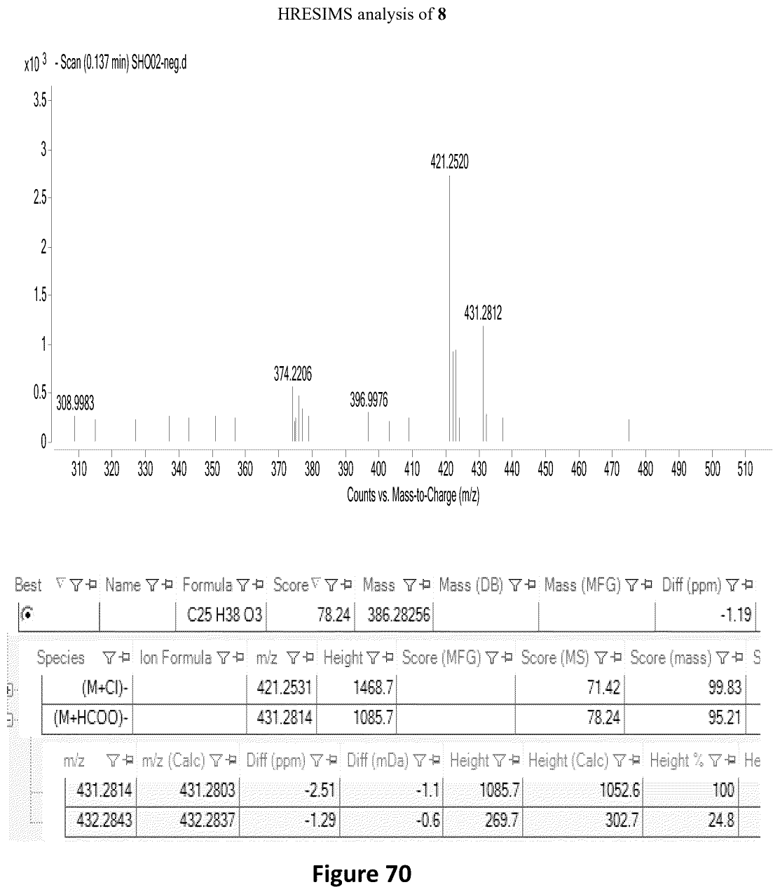

A summary of NMR data for secosuberitenone A (8) is shown in the table of FIG. 63 with NMT spectra and MS data shown in FIGS. 64-70.

### Norsuberitenone A (9)

Norsuberitenone A (9) was isolated as a white crystalline solid with the molecular formula C₁₈H₃₀O₂ deduced from analysis of the protonated molecular at m/z 279.2320 (+)-HRESIMS data. Norsuberitenone A is significantly smaller than previously isolated suberitenones, and the NMR spectra lacked signals associated with the D ring or the acetyl group. Using HMBC correlations from H₃-15, H₃-17 and H₃-18, rings A and B were deduced to be the same as the suberitenone A (with a hydroxyl in place of the acetyl group at C-8), however ring C was determined to have a ketone at C-2 (δ_{C} 215.1) based on HMBC correlations from H₂-1 (δ_{H} 2.21, 1.90), H₂-3 (δ_{H} 2.37, 2.33) and H-4 (δ_{H} 2.05). This position is the tertiary alcohol carbon that bridges to ring D in suberitenone B (11), therefore it is likely 9 forms as a degradation product from this (blue arrows, FIG. 26). The stereochemistry of the chiral centers were assigned the same configuration as the other congeners based on correlations in the NOESY spectrum.

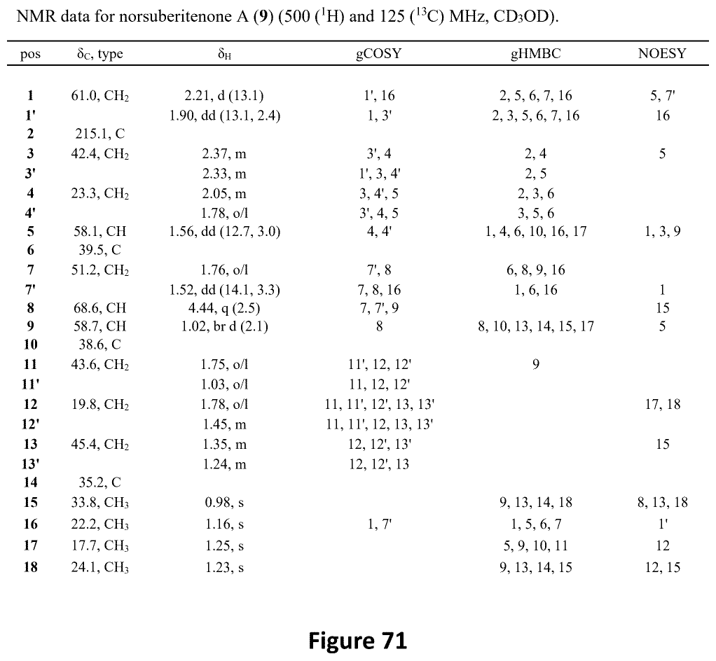

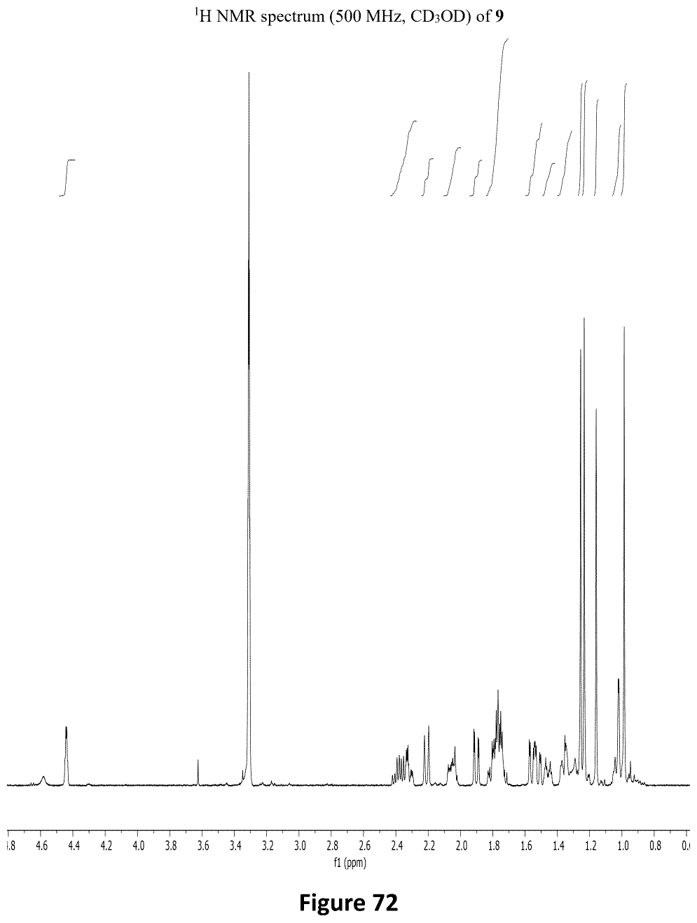

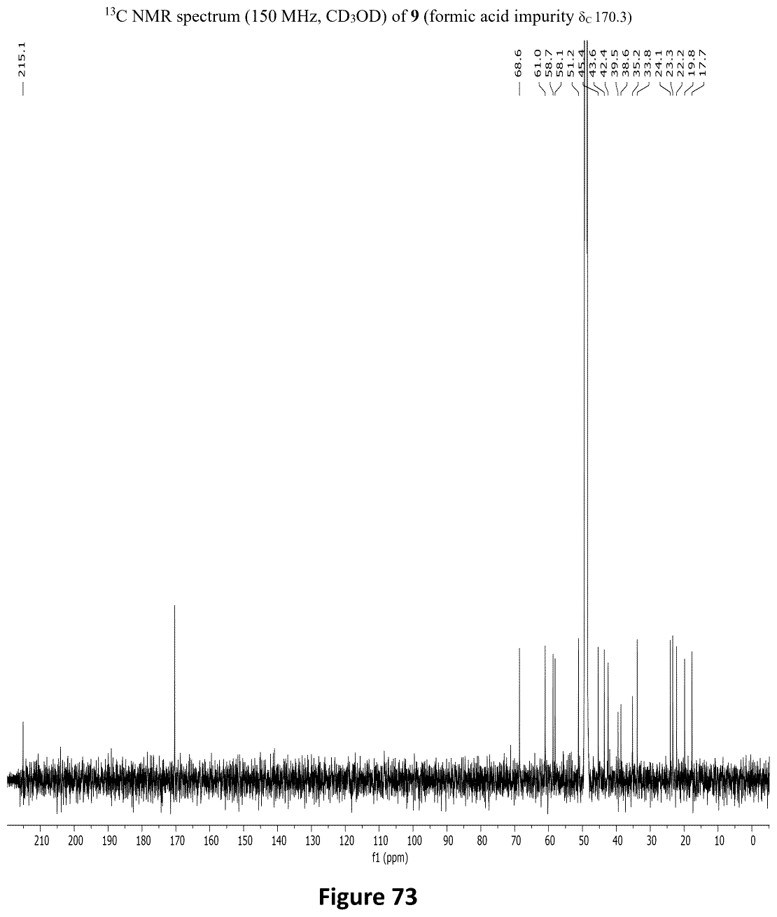

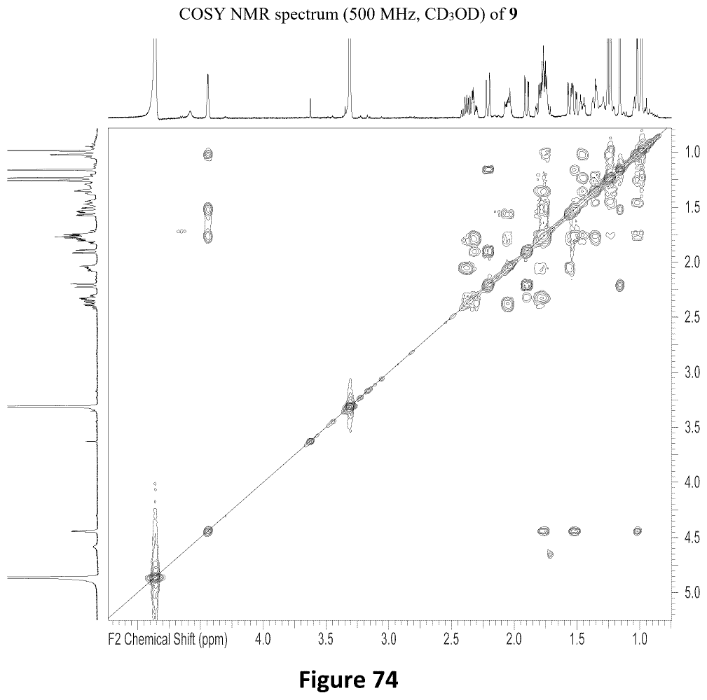

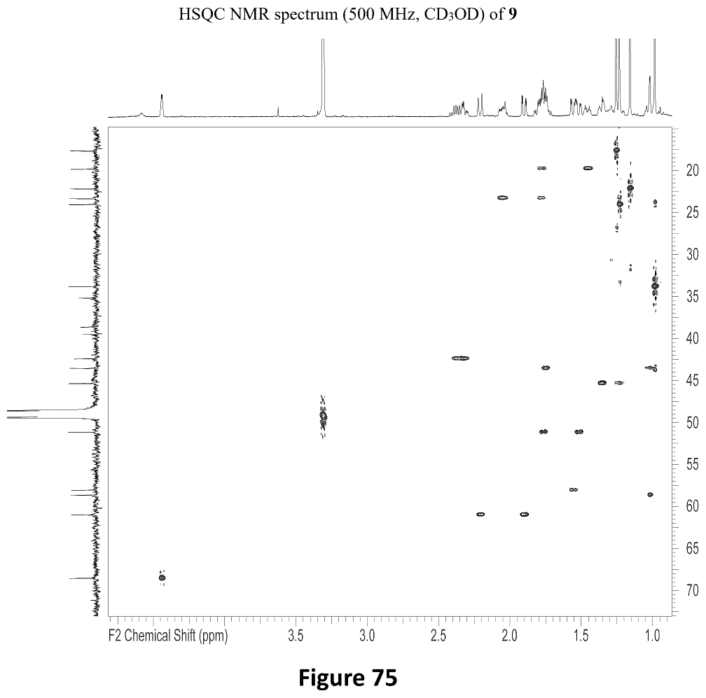

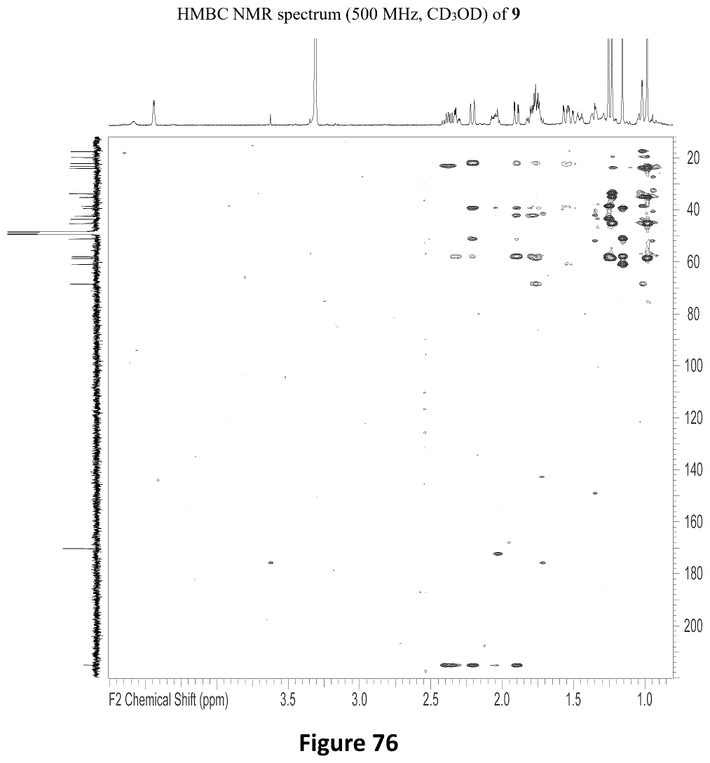

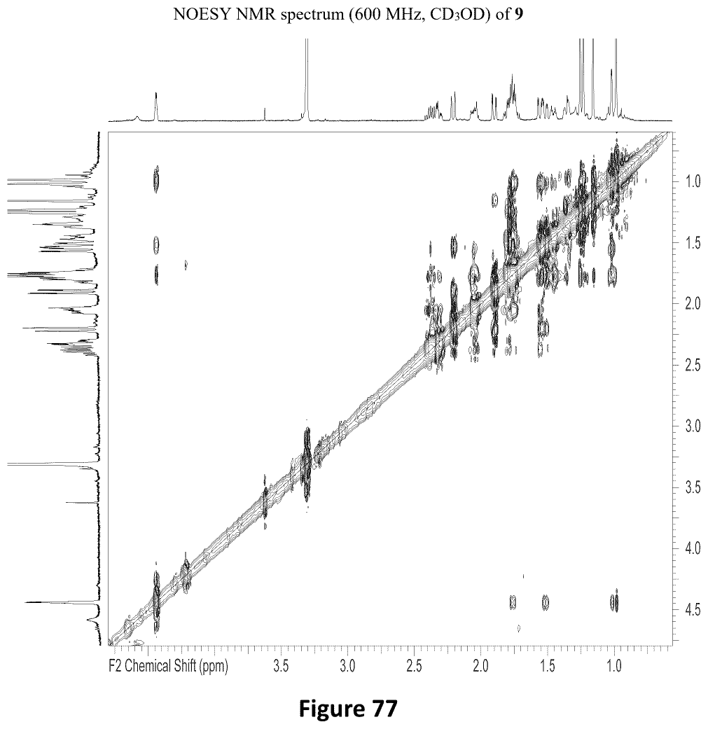

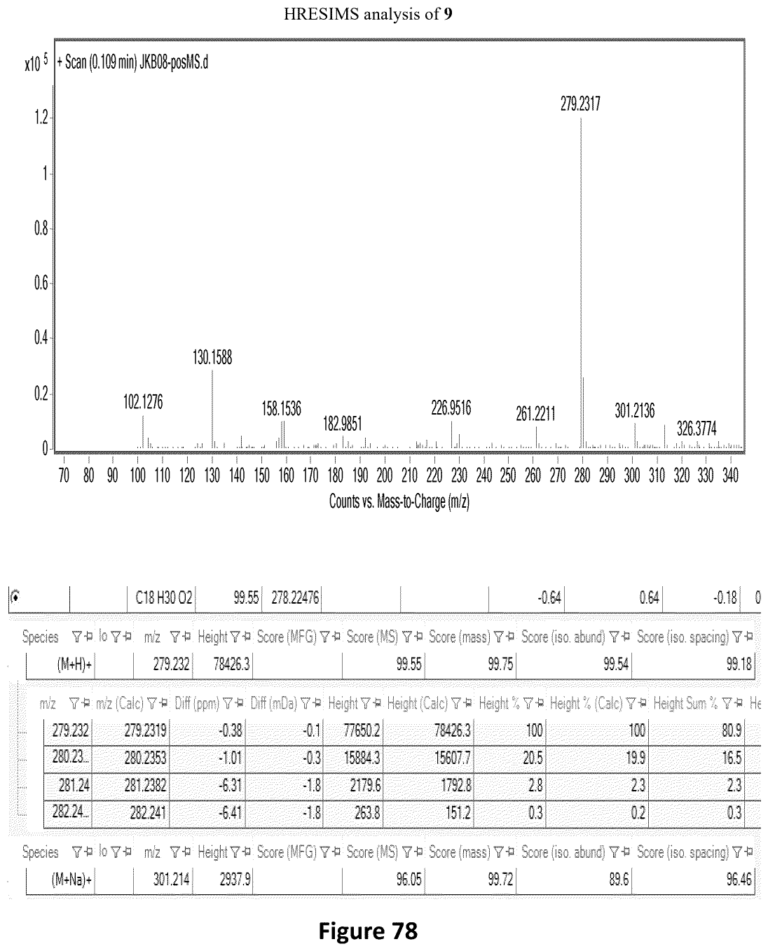

A summary of NMR data for norsuberitenone A (9) is shown in the table of FIG. 71 with NMT spectra and MS data shown in FIGS. 72-78.

### Biogenesis of Metabolites 1-11

A proposed biogenesis of newly isolated metabolites 1 to 11 is presented in FIG. 26. Both 1 and 4 can be envisaged to form from common intermediate 12 (desacetoxy suberitenone C=suberitenol C)¹⁰ by either an intramolecular cyclization or oxidation reaction respectively. Compound 2 forms from suberitenone A (10) by a further epoxidation reaction of the C7/C22 double bond, while 5 is likely a methanol addition product of suberitenone B (11). However, stirring pure isolated suberitenone B in methanol for one week did not produce any detectable amount of 5. Metabolite 9 bearing only 18 carbon atoms is likely a ‘heptanorsesquiterpene’ formed by elimination of the suberitenone B D ring. The purification procedure used avoided the use of ethyl acetate, which proved the acetyl group at C-13 to be biogenerated.

### Materials and Methods

### General Experimental Procedures

Optical rotations were measured using an AutoPol IV polarimeter at 589 nm. UV/Vis spectra were extracted from HPLC chromatograms. NMR spectra were acquired using either a Varian Inova 500 spectrophotometer or a Varian 600 MHz broadband spectrophotometer. The residual solvent peak was used as an internal chemical shift reference (CD₃OD: δ_{C} 49.0; δ_{H} 3.31, CDCl₃: δ_{C} 77.0; δ_{H} 7.26). High-resolution mass spectrometry-liquid chromatography data were obtained on an Agilent 6540 QTOF LCMS with electrospray ionization detection. Reversed-phase HPLC was performed on a Shimadzu LC20-AT system equipped with a photodiode array detector (M20A) using a preparative Phenominex C18 column (5 μm, 100 Å, 250×21.2 mm; 9 mL/min) or on a semipreparative Phenominex C18 column (10 μm, 100 Å, 250×10 mm; 4 mL/min). All solvents used for column chromatography were of HPLC grade, and H₂O was distilled. Solvent mixtures are reported as % v/v unless otherwise stated.

### Biological Material, Extraction, and Isolation

Frozen sponge was freeze dried (1.35 kg wet weight, 200 g dry weight) and then crushed by hand before being extracted in MeOH (1.6 L) twice overnight. The extracts were combined, were then passed through an HP20 column (250 mL), pre-equilibrated in H₂O, and combined following elution. The eluent was then diluted with an equal volume of H₂O and passed back through the column twice, followed by a 750 mL H₂O wash. The column was then eluted with 750 mL portions of (1) 75% Me₂CO/H₂O, and (2) Me₂CO (fractions A1 and A2, respectively). Fraction A1 (5 g) was then reconstituted in MeOH (25 mL), filtered and fractionated generating fractions (B1-B25) by repeated preparative C18 HPLC (9 mL/min) using the following method: 70% ACN/H₂O (0.1% CH₂O₂) for 6 mins, a linear gradient to 100% ACN over 8 mins and 100% ACN isocratic for 11 mins. Fraction B25 contained suberitenone A (˜500 mg), while fraction B23 contained suberitenone B (˜250 mg). Fraction B20 was purified using semipreparative C18 HPLC using a linear gradient from 50% MeOH/H₂O (0.1% CH₂O₂) to 100% MeOH over 25 mins to afford 1 (5.6 mg) and 2 (1.6 mg), while fractions B15, B16, B17, B18, B19 and B21 were also purified by the same method to afford 6 (0.8 mg), 4 (3.4 mg), 8 (0.8 mg) 3 (1.9 mg), 9 (0.8 mg) and 5 (6.5 mg) respectively.

Neosuberitenone A (1): white crystals; [α]²²_{D} 10.5 (c 0.2, MeOH); UV (MeOH/H₂O) λₘₐₓ 210, 235 (sh) nm; ¹H and ¹³C NMR spectra (CDCl₃), see Table 1 and 2; (−)-HRESIMS m/z 473.2915 [M+HCOO]⁻ (calcd for C₂₈H₄₁O₆, 473.2909; Δ 1.32 ppm).

Suberitenone E (2): white crystals; [α]²²_{D} −42 (c 0.1, MeOH); UV (MeOH/H₂O) λₘₐₓ 231 nm; ¹H and ¹³C NMR spectra (CD₃OD), see Table 1 and 2; (−)-HRESIMS m/z 489.2865 [M+HCOO]⁻ (calcd for C₂₈H₄₁O₇, 489.2858; Δ 1.58 ppm).

Suberitenone F (3): white film; [α]²²_{D} −45 (c 0.1, MeOH); UV (MeOH/H₂O) λₘₐₓ 233 nm; ¹H and ¹³C NMR spectra (CD₃OD), see Table 1 and 2; (−)-HRESIMS m/z 489.2841 [M+HCOO]⁻ (calcd for C₂₈H₄₁O₇, 489.2858; Δ 3.34 ppm).

Suberitenone G (4): white film; [α]²²_{D} −62 (c 0.2, MeOH); UV (MeOH/H₂O) λₘₐₓ 233 nm; ¹H and ¹³C NMR spectra (CD₃OD), see Table 1 and 2; (−)-HRESIMS m/z 441.2645 [M−H]⁻ (calcd for C₂₇H₃₇O₅, 441.2646; Δ 0.42 ppm).

Suberitenone H (5): white film; [α]²²_{D} −9.0 (c 0.2, MeOH); UV (MeOH/H₂O) λₘₐₓ 233, 280 (sh) nm; ¹H and ¹³C NMR spectra (CD₃OD), see Table 1 and 2; (−)-HRESIMS m/z 523.3284 [M+HCOO]⁻ (calcd for C₂₉H₄₇O₈, 523.3276; Δ 1.36 ppm).

Suberitenone I (6): white film; [α]²²_{D} 7.7 (c 0.1, MeOH, 3:1 mixture with 7); UV (MeOH/H₂O) λₘₐₓ 210 nm; ¹H and ¹³C NMR spectra (CD₃OD), see Table 1 and 2; (−)-HRESIMS m/z 489.2855 [M+HCOO]⁻ (calcd for C₂₈H₄₁O₇, 489.2858; Δ 0.54 ppm).

Suberitenone J (7): white film; [α]²²_{D} 7.7 (c 0.1, MeOH, 3:1 mixture with 7); UV (MeOH/H₂O) λₘₐₓ 232 nm; ¹H and ¹³C NMR (CD₃OD) could not be fully assigned due to rapid conversion to 6 upon purification. The observed signals that differed from 6 are reported in Table S7; (−)-HRESIMS m/z 489.2855 [M+HCOO]⁻ (calcd for C₂₈H₄₁O₇, 489.2858; Δ 0.54 ppm).

Secosuberitenone A (8): white film; [α]²²_{D} −11.7 (c 0.1, MeOH); UV (MeOH/H₂O) λₘₐₓ 230 nm; ¹H and ¹³C NMR spectra (CDCl₃), see Table 1 and 2; (−)-HRESIMS m/z 431.2814 [M+HCOO]⁻ (calcd for C₂₆H₃₉O₅, 431.2803; Δ −2.51 ppm).

Norsuberitenone A (9): white crystalline solid; [α]²²_{D} 6.3 (c 0.1, MeOH); UV (MeOH/H₂O) λₘₐₓ 210, 237, 285 (sh) nm; ¹H and ¹³C NMR spectra (CD₃OD), see Table S9; (+)-HRESIMS m/z 279.2320 [M+H]⁺ (calcd for C₁₈H₃₁O₂, 279.2319; Δ 0.38 ppm).

### X-Ray Crystallography

Crystallographic data for the structures reported in this article were deposited at the Cambridge Crystallographic Data Center under the deposition numbers CCDC 2164632 (1) and 2164633 (2). Copies of the data can be obtained free of charge via www.ccdc.cam.ac.uk/data_request/cif. XRD data were measured on Bruker D8 Venture PHOTON II CMOS diffractometer equipped with a Cu Kα INCOATEC ImuS microfocus source (λ=1.54178 Å). Indexing was performed using APEX4 (Difference Vectors method).¹⁵ Data integration and reduction were performed using SaintPlus.¹⁶ Absorption correction was performed by multi-scan method implemented in SADABS.¹⁷ Space group was determined using XPREP implemented in APEX3.15 Structure was solved using SHELXT¹⁸ and refined using SHELXL-2018/3 (full-matrix least-squares on F2)¹⁹ through OLEX2 interface program.²⁰ Ellipsoid plot was done with Platon.²¹

Neosuberitenone A (1): Hydrogen atom of —OH group was found from difference Fourier map and was freely refined. All remaining hydrogen atoms were refined using riding model. Crystal data: C₂₇H₄₀O₄, M=428.59 g/mol, monoclinic, space group C2, a=24.7343 (6) Å, b=6.27570 (10) Å, c=18.2166 (4) Å, V=2308.80 (9) Å³, Z=4, T=100.00 K, μ(Cu Kα)=0.636 mm⁻¹, ρ_{calc}=1.233 g/cm³, 21421 reflections measured (5.942°≤2θ≤159.866°), 4852 independent reflections (Rᵢₙₜ=0.0409, R_{sigma}=0.0334), which were used in all calculations. The final R₁ was 0.0330 (I>=2σ(I)) and wR₂ was 0.0881 (all data). Flack parameter: 0.06(6). A full table of these parameters for the crystal structure can be found in the supplementary information.

Suberitenone E (2): Hydrogen atom of —OH group was found from difference Fourier map and was refined with distance restraint. All remaining hydrogen atoms were refined using riding model. Crystal data: C₂₇H₄₀O₅, M=444.59 g/mol, monoclinic, space group P2₁, a=6.7821 (2) Å, b=8.9780 (3) Å, c=20.5457 (6) Å, V=1245.06 (7) Å³, Z=2, T=298.00 K, μ(Cu Kα)=0.638 mm⁻¹, ρ_{calc}=1.186 g/cm³, 28530 reflections measured (8.648°≤2θ≤158.82°), 5185 independent reflections (Rᵢₙₜ=0.0572, R_{sigma}=0.0426), which were used in all calculations. The final R₁ was 0.0465 (I>=2σ(I)) and wR₂ was 0.1325 (all data). Flack parameter: 0.04(11). A full table of these parameters for the crystal structure can be found in the supplementary information.

### Example 2—Treatment of RSV

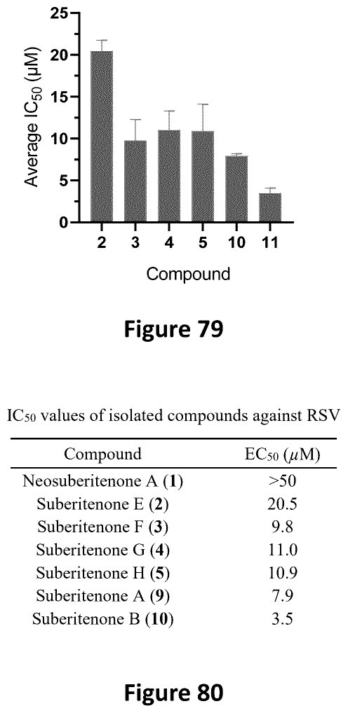

The newly isolated compounds alongside suberitenone A (10) and B (11) were assessed for their ability to inhibit RSV gene expression and their effect on the cell viability (FIG. 79). Neosuberitenone (1) was the only compound that did not show any viral inhibitory effects at 25 μg/mL (6, 8 and 9 not tested due to lack of sample supply), suggesting that ring D must be appended by a single bond to result in activity. Suberitenone A and B demonstrated the strongest antiviral potential of the tested compounds, with IC₅₀ values of 7.9 and 3.5 μM respectively. The other isolated compounds had IC₅₀ values ranging from 9.8-20.5 μM (FIG. 80), therefore suggesting oxidation about ring C and removing the enone double bond of ring D are not likely to be alterations that would result in an increase in activity for future structure-activity relationship studies. None of the compounds reached 50% cytotoxicity against A549 cells at the highest concentrations tested (100 μM, data not shown).

Suberites sp. compounds were also submitted to a broad range of infectious disease screening. No antibacterial activity was detected against the ESKAPE pathogens, nor antifungal activity against several Candida strains. The cytotoxicity was assayed against the murine macrophage J774 cell line, where no significant activity (EC₅₀ below 10 μM) was observed for any compound. Taken with low cytotoxicity, the selectivity of suberitenones A and B (10, 11) for RSV among other infectious diseases is noteworthy.

### Materials and Methods

### RSV Antiviral Assay

A549 (CCL-185, ATCC) cells were seeded at 1.6×10⁴ cells in 100 μl of 5% FBS/1× penicillin-streptomycin/F12 medium per well in 96-well uClear® black plates with clear bottom (Greiner 655090). Cells were infected with 400 PFU/well of a recombinant RSV encoding Renilla luciferase as an additional transcription unit (rA2-Rluc²²) in total volume of 50 μL/well and allowed to adsorb for 1 h. Purified compounds were serially diluted two-fold in triplicate and 50 μL/well of the diluted extracts were added to the infected cells. After further incubation at 37° C. and 5% CO₂ for 24 h, the supernatants were removed and cells were lysed using Renilla lysis buffer (Promega). Luciferase activity was measured with the Renilla Luciferase reagent (Promega) and BioTek Synergy Mx microplate luminometer using Gen5 version 2.00.18 software. RSV antiviral effect for test samples was determined by normalizing to DMSO-treated control samples and multiplying by 100 to obtain percent of control. Statistical analysis was performed using GraphPad Prism software version 9.4.1.

### Cytotoxicity Assay

To test for cell viability, duplicate 96-well plates of uninfected A549 cells were set up in parallel with the RSV antiviral assay. Extracts were diluted and used to treat cells as described above for the RSV antiviral assay. The negative control for cytotoxicity was 2% DMSO while the positive was 2 μM (final concentration) of PDKI inhibitor. After the 24 h incubation period, cells were processed for MTT Proliferation Assay (Provost & Wallert Research) according to the manufacturer's protocol. Absorbance at 570 nm was measured by microplate luminometer and standardized to control as above. 2% DMSO and 2 μM PDK1 inhibitor (CalBioChem) were used as negative and positive controls for cell death, respectively. Statistical analysis was performed using GraphPad Prism software version 9.4.1.

### Example 3—Treatment of RSV (Prophetic)

A 45-year-old female presents with runny nose, sore throat, and cough. The patient is diagnosed with RSV. The patient is orally administered a therapeutically effective amount of a pharmaceutical composition containing synthetic compound 10 and a pharmaceutically acceptable carrier. Improvement is seen in the symptoms after treatment over a given time period.

A 35-year-old female presents with cough and headache. The patient is diagnosed with RSV. The patient is orally administered a therapeutically effective amount of a pharmaceutical composition containing synthetic compound 11 and a pharmaceutically acceptable carrier. Improvement is seen in the symptoms after treatment over a given time period.

A 50-year-old male presents with runny nose, fever, and cough. The patient is diagnosed with RSV. The patient is orally administered a therapeutically effective amount of a pharmaceutical composition containing synthetic compound 3 and a pharmaceutically acceptable carrier. Improvement is seen in the symptoms after treatment over a given time period.

### CONCLUSION

A ¹H NMR guided fractionation procedure of the polar extract constituents of the Antarctic sponge Suberites sp. resulted in the isolation of nine new sesterterpenoids; neosuberitenone A (1), suberitenones E-J (2-7), secosuberitenone A (8) and norsuberitenone A (9) along with large quantities of suberitenone A (10) and B (11). The previously unreported compounds showed various oxidations and ring formations never reported from this class of compounds, including the new neosuberitane terpenoid carbon skeleton solved by a combination of NMR interpretation and XRD data. Although the compounds did not show activity in antibacterial or antifungal assays, they showed the ability to inhibit RSV viral transcription with relatively low levels of cytotoxicity. The new compounds demonstrated weaker activity that suberitenones A and B and provides important information future structure activity relationship studies and structure optimization.

### REFERENCES

1. Falsey, A. R.; Hennessey, P. A.; Formica, M. A.; Cox, C.; Walsh, E. E., Respiratory syncytial virus infection in elderly and high-risk adults. N. Engl. J. Med. 2005, 352, 1749-1759.
2. Ali, A.; Lopardo, G.; Scarpellini, B.; Stein, R. T.; Ribeiro, D., Systematic review on respiratory syncytial virus epidemiology in adults and the elderly in Latin America. Int. J. Infect. Dis. 2020, 90, 170-180.
3. Behzadi, M. A.; Leyva-Grado, V. H., Overview of current therapeutics and novel candidates against influenza, respiratory syncytial virus, and middle east respiratory syndrome Coronavirus infections. Front. Microbiol. 2019, 10, 1327.
4. Sun, Z.; Pan, Y.; Jiang, S.; Lu, L., Respiratory Syncytial Virus entry inhibitors targeting the F protein. Viruses 2013, 5, 211-225.
5. Domachowske, J. B.; Anderson, E. J.; Goldstein, M., The future of respiratory syncytial virus disease prevention and treatment. Infect. Dis. Ther. 2021, 10, 47-60.
6. Ma, W. S.; Mutka, T.; Vesley, B.; Amsler, M. O.; Mcclintock, J. B.; Amsler, C. D.; Perman, J. A.; Singh, M. P.; Maiese, W. M.; Zaworotko, M. J.; Kyle, D. E.; Baker, B. J., Norselic acids A-E, highly oxidized anti-infective steroids that deter mesograzer predation, from the Antarctic sponge Crella sp. J. Nat. Prod. 2009, 72, 1842-1846.
7. Knestrick, M. A.; Wilson, N. G.; Roth, A.; Adams, J. H.; Baker, B. J., Friomaramide, a highly modified linear hexapeptide from an Antarctic sponge, inhibits Plasmodium falciparum liver-stage development. J. Nat. Prod. 2019, 82, 2354-2358.
8. Shilling, A. J.; Witowski, C. G.; Maschek, J. A.; Azhari, A.; Vesely, B. A.; Kyle, D. E.; Amsler, C. D.; Mcclintock, J. B.; Baker, B. J., Spongian diterpenoids derived from the Antarctic sponge Dendrilla antarctica are potent inhibitors of the Leishmania parasite. J. Nat. Prod. 2020, 83, 1553-1562.
9. Shin, J.; Seo, Y.; Rho, J.-R.; Baek, E.; Kwon, B.-M.; Jeong, T.-S.; Bok, S.-H., Suberitenones A and B: Sesterterpenoids of an unprecedented skeletal class from the Antarctic sponge Suberites sp. J. Org. Chem. 1995, 60, 7582-7588.
10. Lee, H.-S.; Ahn, J.-W.; Lee, Y.-H.; Rho, J.-R.; Shin, J., New sesterterpenes from the Antarctic sponge Suberites sp. J. Nat. Prod. 2004, 67, 672-674.
11. Diaz-Marrero, A. R.; Brito, I.; Cueto, M.; San-Martin, A.; Darias, J., Suberitane network, a taxonomical marker for Antarctic sponges of the genus Suberites? Novel sesterterpenes from Suberites caminatus. Tetrahedron Lett. 2004, 45, 4707-4710.
12. Solanki, H.; Angulo-Preckler, C.; Calabro, K.; Kaur, N.; Lasserre, P.; Cautain, B.; de la Cruz, M.; Reyes, F.; Avila, C.; Thomas, O. P., Suberitane sesterterpenoids from the Antarctic sponge Phorbas areolatus (Thiele, 1905). Tetrahedron Lett. 2018, 59, 3353-3356.
13. Wang, M.; Tietjen, I.; Chen, M.; Williams, D. E.; Daoust, J.; Brockman, M. A.; Andersen, R. J., Sesterterpenoids isolated from the sponge Phorbas sp. activate latent HIV-1 provirus expression. J. Org. Chem. 2016, 81, 11324-11334.
14. Daoust, J.; Fontana, A.; Merchant, C. E.; de Voogd, N. J.; Patrick, B. O.; Kieffer, T. J.; Andersen, R. J., Ansellone A, a sesterterpenoid isolated from the nudibranch Cadlina luteromarginata and the sponge Phorbas sp., activates the cAMP signaling pathway. Org. Lett. 2010, 12, 3208-3211.
15. Bruker APEX4 (2022), Bruker AXS LLC: Madison, Wisconsin, USA.
16. Bruker SAINT (2022), Bruker AXS LLC: Madison, Wisconsin, USA.
17. Krause, L.; Herbst-Irmer, R.; Sheldrick, G. M.; Stalke, D., Comparison of silver and molybdenum microfocus X-ray sources for single-crystal structure determination. J. Appl. Cryst. 2015, 48, 3-10.
18. Sheldrick, G., SHELXT-Integrated space-group and crystal-structure determination. Acta Cryst. A 2015, 71, 3-8.
19. Sheldrick, G., Crystal structure refinement with SHELXL. Acta Cryst. C 2015, 71, 3-8.
20. Dolomanov, O. V.; Bourhis, L. J.; Gildea, R. J.; Howard, J. A. K.; Puschmann, H., OLEX2: a complete structure solution, refinement and analysis program. J. Appl. Cryst. 2009, 42, 339-341.
21. Spek, A., Single-crystal structure validation with the program PLATON. J. Appl. Cryst. 2003, 36, 7-13.
22. Fuentes, S.; Crim, R. L.; Beeler, J.; Teng, M. N.; Golding, H.; Khurana, S., Development of a simple, rapid, sensitive, high-throughput luciferase reporter based microneutralization test for measurement of virus neutralizing antibodies following respiratory syncytial virus vaccination and infection. Vaccine 2013, 31, 3987-3994.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of treating a viral respiratory infection in a patient in need thereof comprising:
administering to the patient a therapeutically effective amount of a pharmaceutical composition comprising
at least one compound selected from the group consisting of compound 2, compound 3, compound 4, compound 5, compound 6, compound 7, compound 8, compound 9, compound 10, compound 11, and pharmaceutically acceptable salts thereof; and
a pharmaceutically acceptable carrier;
wherein the therapeutically effective amount of the composition treats the respiratory infection of the patient.

2. The method of claim 1, wherein the virus is from the Paramyxovirdae family.

3. The method of claim 2, wherein the virus causing the viral respiratory infection is selected from the group consisting of respiratory syncytial virus (RSV), human metapneumovirus (HMPV), and parainfluenza virus.

4. The method of claim 3, wherein the virus is RSV.

5. The method of claim 1, wherein the compound is compound 3, compound 10, compound 11, or a pharmaceutically acceptable salt thereof.

6. The method of claim 5, wherein the at least one compound is compound 10 or a pharmaceutically acceptable salt thereof.

7. The method of claim 5, wherein the compound is compound 11 or a pharmaceutically acceptable salt thereof.

8. A pharmaceutical composition comprising:
at least one synthetic compound selected from the group consisting of compound 1, compound 2, compound 3, compound 4, compound 5, compound 6, compound 7, compound 8, compound 9, and pharmaceutically acceptable salts thereof; and
a pharmaceutically acceptable carrier.

9. The composition of claim 8, wherein the at least one compound is compound 3.

10. A method of inhibiting viral gene expression in a cell comprising:
contacting at least one cell with a therapeutically effective amount of at least one compound selected from the group consisting of compound 2, compound 3, compound 4, compound 5, compound 6, compound 7, compound 8, compound 9, compound 10, compound 11, and pharmaceutically acceptable salts thereof;
wherein the at least one compound inhibits viral gene transcription in the cell.

11. The method of claim 10, wherein the viral gene is from a virus from the Paramyxovirdae family.

12. The method of claim 11, wherein the virus causing the viral respiratory infection is selected from the group consisting of respiratory syncytial virus (RSV), human metapneumovirus (HMPV), and parainfluenza virus.

13. The method of claim 12, wherein the virus is RSV.

14. The method of claim 10, wherein the compound is compound 3, compound 10, compound 11, or a pharmaceutically acceptable salt thereof.

15. The method of claim 10, wherein the at least one compound is compound 10 or a pharmaceutically acceptable salt thereof.

16. The method of claim 10, wherein the compound is compound 11 or a pharmaceutically acceptable salt thereof.
